(19) United States
(12) Patent Application Publication
(10) Pub. No.: US 2025/0042861 A1
(43) Pub. Date: Feb. 6, 2025

(54) TETRAHYDROPYRIDAZINES, COMPOSITIONS COMPRISING THEM AND USES THEREOF

(71) Applicant: Novo Nordisk A/S, Bagsvaerd (DK)

(72) Inventors: Jeremy Green, Waltham, MA (US); Shawn Gallagher-Duval, Montreal (CA); Alexandre Lemire, Montreal (CA); Francois Ravenelle, Montreal (CA)

(21) Appl. No.: 18/718,578

(22) PCT Filed: Dec. 16, 2022
(86) PCT No.: PCT/CA2022/051841

§ 371 Date: Jun. 11, 2024

Related U.S. Application Data
(60) Provisional application No. 63/265,583, filed on Dec. 17, 2021.

(51) Int. Cl. C07D 237/04; A61K 31/50; A61K 31/501; C07D 401/12

(52) U.S. Cl. CPC C07D 237/04; A61K 31/50; A61K 31/501; C07D 401/12

(57) ABSTRACT

The present document relates to tetrahydropyridazine compounds, pharmaceutical compositions comprising the same and their use in the treatment or prevention of diseases and disorders associated with the cannabinoid CB₁ receptor. For example, the tetrahydropyridazine compounds, or a tautomeric form and/or salt thereof, are of Formula I, wherein R¹ to R⁴, X¹, and a are as defined herein.

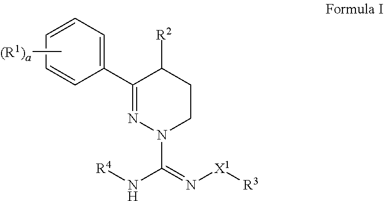

### RELATED APPLICATION

The present application claims priority under applicable law to U.S. provisional application No. 63/265,583 filed on Dec. 17, 2021, the content of which is incorporated herein by reference in its entirety for all purposes.

### TECHNICAL FIELD

This disclosure generally relates to compounds, especially tetrahydropyridazine compounds, pharmaceutical compositions comprising them and their use and methods of use in the treatment and prevention of diseases and disorders.

### BACKGROUND

It is generally known that activation of the cannabinoid CB₁ receptor increases appetite, increases the biosynthesis and storage of lipids, inhibits the actions of insulin and leptin, and promotes inflammation and fibrosis. Research was thus focused on developing CB₁ receptor inhibitors for the potential treatment of obesity and the metabolic disorder associated therewith, referred to as metabolic syndrome. Rimonabant was shown effective in treating metabolic syndrome but caused neuropsychiatric (i.e. CNS-related) side effects, which resulted in its withdrawal from the market.

There remains a need for the development of alternative compounds targeting the CB₁ receptor for the treatment or prevention of disorders associated thereto.

### SUMMARY

According to one aspect, the present technology relates to compounds and their tautomeric forms and/or pharmaceutically acceptable salt thereof, their pharmaceutical compositions, uses thereof and methods of treatment comprising their administration. More specifically, the following embodiments are provided:
Embodiment 1. A compound of Formula I:

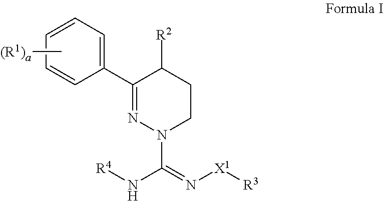

wherein,
R¹ is independently in each occurrence selected from optionally substituted alkyl, optionally substituted cycloalkyl, optionally substituted heterocycloalkyl, halogen, cyano, nitro, hydroxy, optionally substituted alkoxy, amino, optionally substituted alkylsulfonyl, optionally substituted aryl, optionally substituted heteroaryl, optionally substituted carboxyl, acyl, optionally substituted alkenyl, optionally substituted alkynyl, optionally substituted phosphonyl, optionally substituted phosphinyl, optionally substituted boronate, optionally substituted silyl, and imino;
R² and R³ are each independently selected from optionally substituted alkyl, aryl, heteroaryl, cycloalkyl, and heterocycloalkyl groups;
R⁴ is selected from —C(O)R⁵, —S(O)₂R5, —C(═NR⁶)R⁵, and —C(═NR⁶)NHC(O)R⁵;
R⁵ is selected from optionally substituted alkyl, optionally substituted alkoxy, optionally substituted alkylamino or dialkylamino, optionally substituted cycloalkyl, optionally substituted heterocycloalkyl, optionally substituted aryl, and optionally substituted heteroaryl;
R⁶ is selected from hydrogen, optionally substituted alkyl, optionally substituted alkoxy, optionally substituted alkylamino or dialkylamino, optionally substituted cycloalkyl, optionally substituted heterocycloalkyl, optionally substituted aryl, and optionally substituted heteroaryl, or R⁶ is taken together with R⁵ and their adjacent atoms to form an optionally substituted heterocycloalkyl or heteroaryl;
X¹ is selected from SO₂ and C═O; and
a is 0, 1, 2, 3, 4 or 5;
or an isomer and/or tautomer thereof, or a pharmaceutically acceptable salt thereof.
Embodiment 2. The compound of embodiment 1, wherein said compound is of Formula I(a):

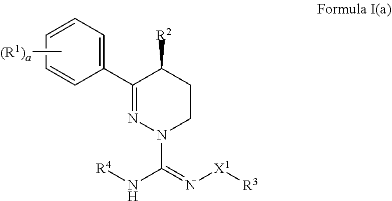

wherein R¹ to R⁴, X¹, and a are as defined in embodiment 1.
Embodiment 3. The compound of embodiment 1, wherein said compound is of Formula I(b):

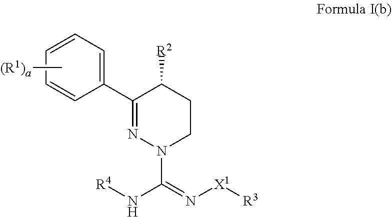

wherein R¹ to R⁴, X¹, and a are as defined in embodiment 1.
Embodiment 4. The compound of any one of embodiments 1 to 3, wherein R⁴ is —C(O)R⁵, —C(═NR⁶)R⁵, or —C(═NR⁶)NHC(O)R⁵.
Embodiment 5. The compound of embodiment 4, wherein R⁴ is —C(═NR⁶)NHC(O)R⁵.
Embodiment 6. The compound of any one of embodiments 1 to 5, wherein R⁵ is an optionally substituted C₁₋₆alkyl, optionally substituted C₁₋₆alkoxy, optionally substituted C₁₋₆ alkylamino or di C₁₋₆alkylamino.
Embodiment 7. The compound of embodiment 6, wherein R⁵ is C₁₋₆alkyl, e.g. methyl.
Embodiment 8. The compound of any one of embodiments 1 to 5, wherein R⁵ is an optionally substituted cycloalkyl, optionally substituted heterocycloalkyl, optionally substituted aryl, or optionally substituted heteroaryl.
Embodiment 9. The compound of any one of embodiments 1 to 8, wherein R⁶ is H.
Embodiment 10. The compound of any one of embodiments 1 to 5, wherein R⁶ is taken together with R⁵ and their adjacent atoms to form an optionally substituted heterocycloalkyl or heteroaryl.
Embodiment 11. The compound of any one of embodiments 1 to 10, wherein X¹ is SO₂.
Embodiment 12. The compound of any one of embodiments 1 to 11, wherein a is 1 and R¹ is halogen.
Embodiment 13. The compound of embodiment 12, wherein R¹ is Cl.
Embodiment 14. The compound of any one of embodiments 1 to 13, wherein R³ is an optionally substituted C₂₋₆alkyl group.
Embodiment 15. The compound of any one of embodiments 1 to 13, wherein R³ is an optionally substituted C₆aryl, C₅₋₆heteroaryl or C₅₋₇heterocycloalkyl group.
Embodiment 16. The compound of embodiment 15, wherein R³ is of the formula:

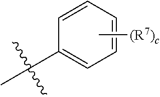

wherein,
R⁷ is independently in each occurrence selected from halogen, OH, CN, NO₂, C(O)R⁹, C(O)N(R⁸)₂, C(R¹⁰)═NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, OC₁₋₆alkyl, C₅₋₁₀aryl, C₅₋₁₀heteroaryl, C₃₋₁₀cycloalkyl, and C₄₋₁₀heterocycloalkyl;
R⁸ is independently in each occurrence selected from H, C₁₋₆alkyl, C₂₋₆alkenyl, C₂₋₆alkynyl, C₃₋₁₀cycloalkyl, C₄₋₁₀heterocycloalkyl, C₆aryl, and C₅₋₁₀heteroaryl, or two R⁸ are taken together with their adjacent atom(s) to form a C₄₋₁₀heterocycloalkyl group;
R⁹ is independently in each occurrence selected from C₁₋₆alkyl, C₂₋₆alkenyl, C₂₋₆alkynyl, C₃₋₇cycloalkyl, C₆aryl, and C₅₋₆heteroaryl; and
R¹⁰ is independently in each occurrence selected from H, C₁₋₆alkyl, C₂₋₆alkenyl, C₂₋₆alkynyl, C₃₋₇-cycloalkyl, C₆aryl, and C₅₋₆heteroaryl; and
c is 0, 1, 2, 3, 4 or 5;
wherein said alkyl, alkenyl, alkynyl, cycloalkyl, heterocycloalkyl, aryl or heteroaryl group is optionally further substituted.
Embodiment 17. The compound of embodiment 15, wherein R³ is of the formula:

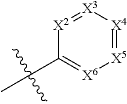

wherein,
X², X³, X⁴, X⁵, and X⁶ are each independently selected from N and CR¹¹, wherein at most three of X², X³, X⁴, X⁵, and X⁶ are N.
R¹¹ is independently in each occurrence selected from hydrogen, halogen, OH, CN, NO₂, C(O)R⁹, C(O)N(R⁸)₂, C(R¹⁰)═NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, OC₁₋₆alkyl, C₆-10aryl, C₅₋₁₀heteroaryl, C₃₋₁₀cycloalkyl, and C₄₋₁₀heterocycloalkyl; and
c is 0, 1, 2, 3, 4 or 5;
wherein R⁸, R⁹, and R¹⁰ are as defined in embodiment 16; and
wherein said alkyl, alkenyl, alkynyl, cycloalkyl, heterocycloalkyl, aryl or heteroaryl group is optionally further substituted.
Embodiment 18. The compound of embodiment 15, wherein R³ is of the formula:

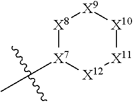

wherein,
X⁷ is selected from N and CR¹¹;
X⁸, X⁹, X¹⁰, X¹¹, and X¹² are each independently selected from O, NR^{x} and C(R^{y})₂, wherein at most two of X⁷, X⁸, X⁹, X¹⁰, X¹¹, and X¹² are or comprise 0 or N;
R¹¹ is as defined in embodiment 17;
R^{x} is selected from is independently in each occurrence selected from hydrogen, C(O)R⁹, C(O)N(R⁸)₂, SO₂R⁹, SO₂N(R⁸)₂, P(O)(R⁸)₂, P(O)(OR⁸)₂, B(OR⁸)₂, C₁₋₆alkyl, C₂₋₆alkenyl, C₂₋₆alkynyl, C₆-10aryl, C₅₋₁₀heteroaryl, C₃₋₁₀cycloalkyl, and C₄₋₁₀heterocycloalkyl;
R^{y} is independently in each occurrence selected from hydrogen, halogen, OH, CN, NO₂, C(O)R⁹, C(O)N(R⁸)₂, C(R¹⁰)═NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, OC₁₋₆alkyl, C₆-10aryl, C₅₋₁₀heteroaryl, C₃₋₁₀cycloalkyl, and C₄₋₁₀heterocycloalkyl or two R^{y} are taken together with their adjacent atom(s) to form a C₄₋₁₀cycloalkyl or C₄₋₁₀heterocycloalkyl group;
wherein R⁸, R⁹, and R¹⁰ are as defined in embodiment 16; and
wherein said alkyl, alkenyl, alkynyl, cycloalkyl, heterocycloalkyl, aryl or heteroaryl group is optionally further substituted.
Embodiment 19. The compound of embodiment 17 or 18, wherein R¹¹ or R^{y} is a halogen (e.g. F or C1) or halogenated C₁₋₆alkyl (e.g. fluorinated C₁-3alkyl), in at least one occurrence.
Embodiment 20. The compound of embodiment 17 or 18, wherein R¹¹ or R^{y} is a hydrogen atom in each occurrence.
Embodiment 21. The compound of any one of embodiments 1 to 20, wherein R² is an optionally substituted C₂₋₆alkyl group.
Embodiment 22. The compound of any one of embodiments 1 to 20, wherein R² is an optionally substituted C₆aryl, C₅₋₆heteroaryl or C₅₋₇heterocycloalkyl group.
Embodiment 23. The compound of embodiment 22, wherein R² is of the formula:

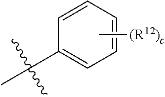

wherein,
R¹² is independently in each occurrence selected from halogen, OH, CN, NO₂, C(O)R¹⁴, C(O)N(R¹³)₂, C(R¹⁵)═NR¹⁵, SO₂R¹⁴, SO₂N(R¹³)₂, N(R¹⁵)C(O)R¹⁴, N(R¹⁵)SO₂R¹⁴, N(R¹⁵)C(O)N(R¹³)₂, N(R¹⁵)SO₂N(R¹³)₂, N(R¹³)₂, P(O)(R¹³)₂, P(O)(OR¹³)₂, B(OR¹³)₂, C₁₋₆alkyl, C₂₋₆alkenyl, C₂₋₆alkynyl, OC₁₋₆alkyl, C₆-10aryl, C₅₋₁₀heteroaryl, C₃₋₁₀cycloalkyl, and C₄₋₁₀heterocycloalkyl;
R¹³ is independently in each occurrence selected from H, C₁₋₆alkyl, C₂₋₆alkenyl, C₂₋₆alkynyl, C₃₋₁₀cycloalkyl, C₄₋₁₀heterocycloalkyl, C₆aryl, and C₅₋₁₀heteroaryl, or two R$are taken together with their adjacent atom(s) to form a C₄₋₁₀heterocycloalkyl group;
R¹⁴ is independently in each occurrence selected from C₁₋₆alkyl, C₂₋₆alkenyl, C₂₋₆alkynyl, C₃₋₇cycloalkyl, C₆aryl, and C₅₋₆heteroaryl; and
R¹⁵ is independently in each occurrence selected from H, C₁₋₆alkyl, C₂₋₆alkenyl, C₂₋₆alkynyl, C₃₋₇-cycloalkyl, C₆aryl, and C₅₋₆heteroaryl; and
c is 0, 1, 2, 3, 4 or 5;
wherein said alkyl, alkenyl, alkynyl, cycloalkyl, heterocycloalkyl, aryl or heteroaryl group is optionally further substituted.
Embodiment 24. The compound of embodiment 22, wherein R² is of the formula:

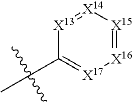

wherein,
X¹³, X¹⁴, X¹⁵, X¹⁶, and X¹⁷ are each independently selected from N and CR¹⁶, wherein at most three of X¹³, X¹⁴, X¹⁵, X¹⁶, and X¹⁷ are N.

R¹⁶ is independently in each occurrence selected from hydrogen, halogen, OH, CN, NO₂, C(O)R¹⁴, C(O)N(R¹³)₂, C(R¹⁵)═NR¹⁵, SO₂R¹⁴, SO₂N(R¹³)₂, N(R¹⁵)C(O)R¹⁴, N(R¹⁵)SO₂R¹⁴, N(R¹⁵)C(O)N(R¹³)₂, N(R¹⁵)SO₂N(R¹³)₂, N(R¹³)₂, P(O)(R¹³)₂, P(O)(OR¹³)₂, B(OR¹³)₂, C₁₋₆alkyl, C₂₋₆alkenyl, C₂₋₆alkynyl, OC₁₋₆alkyl, C₆-10aryl, C₅₋₁₀heteroaryl, C₃₋₁₀cycloalkyl, and C₄₋₁₀heterocycloalkyl; and
c is 0, 1, 2, 3, 4 or 5;
wherein R¹³, R¹⁴, and R¹⁵ are as defined in embodiment 23; and
wherein said alkyl, alkenyl, alkynyl, cycloalkyl, heterocycloalkyl, aryl or heteroaryl group is optionally further substituted.
Embodiment 25. The compound of embodiment 22, wherein R² is of the formula:

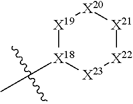

wherein,
X¹⁸ is selected from N and CR¹⁶
X¹⁹, X²⁰, X²1, X²², and X²³ are each independently selected from O, NR^{w} and C(R^{z})₂, wherein at most two of X¹⁸, X¹⁹, X²⁰, X²¹, X²², and X²³ are or comprise 0 or N;
wherein R¹⁶ is as defined in embodiment 24;
R^{w} is selected from is independently in each occurrence selected from hydrogen, C(O)R¹⁴, C(O)N(R¹³)₂, SO₂R¹⁴, SO₂N(R¹³)₂, P(O)(R¹³)₂, P(O)(OR¹³)₂, B(OR¹³)₂, C₁₋₆alkyl, C₂₋₆alkenyl, C₂₋₆alkynyl, C₆-10aryl, C₅₋₁₀heteroaryl, C₃₋₁₀cycloalkyl, and C₄₋₁₀heterocycloalkyl;
R^{z} is independently in each occurrence selected from hydrogen, halogen, OH, CN, NO₂, C(O)R¹⁴, C(O)N(R¹³)₂, C(R¹⁵)═NR¹⁵, SO₂R¹⁴, SO₂N(R¹³)₂, N(R¹⁵)C(O)R¹⁴, N(R¹⁵)SO₂R¹⁴, N(R¹⁵)C(O)N(R¹³)₂, N(R¹⁵)SO₂N(R¹³)₂, N(R¹³)₂, P(O)(R¹³)₂, P(O)(OR¹³)₂, B(OR¹³)₂, C₁₋₆alkyl, C₂₋₆alkenyl, C₂₋₆alkynyl, OC₁₋₆alkyl, C₆-10aryl, C₅₋₁₀heteroaryl, C₃₋₁₀cycloalkyl, and C₄₋₁₀heterocycloalkyl or two R^{z} are taken together with their adjacent atom(s) to form a C₄₋₁₀cycloalkyl or C₄₋₁₀heterocycloalkyl group;
wherein R¹³, R¹⁴, and R¹⁵ are as defined in embodiment 23; and
wherein said alkyl, alkenyl, alkynyl, cycloalkyl, heterocycloalkyl, aryl or heteroaryl group is optionally further substituted.
Embodiment 26. The compound of embodiment 24 or 25, wherein R¹⁶ or R^{z} is a hydrogen atom in each occurrence.
Embodiment 27. The compound of embodiment 24 or 25, wherein R¹⁶ or R^{z} is a halogen (e.g. F or C1) or halogenated C₁₋₆alkyl (e.g. fluorinated C₁-3alkyl), in at least one occurrence.
Embodiment 28. The compound of embodiment 1, wherein the compound is selected from:

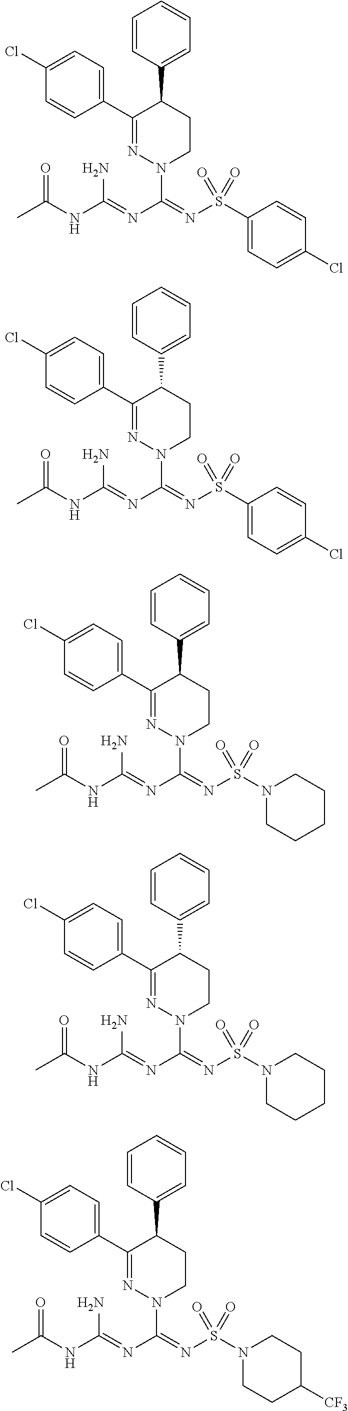 [SMILES: CC(=O)N/C(N)=N/C(=N/S(=O)(=O)c1ccc(Cl)cc1)N1CC[C@H](c2ccccc2)C(c2ccc(Cl)cc2)=N1 ; CC(=O)N/C(N)=N/C(=N/S(=O)(=O)c1ccc(Cl)cc1)N1CC[C@@H](c2ccccc2)C(c2ccc(Cl)cc2)=N1 ; CC(=O)N/C(N)=N/C(=N/S(=O)(=O)N1CCCCC1)N1CC[C@H](c2ccccc2)C(c2ccc(Cl)cc2)=N1 ; CC(=O)N/C(N)=N/C(=N/S(=O)(=O)N1CCCCC1)N1CC[C@@H](c2ccccc2)C(c2ccc(Cl)cc2)=N1 ; CC(=O)N/C(N)=N/C(=N/S(=O)(=O)N1CCC(C(F)(F)F)CC1)N1CC[C@H](c2ccccc2)C(c2ccc(Cl)cc2)=N1]

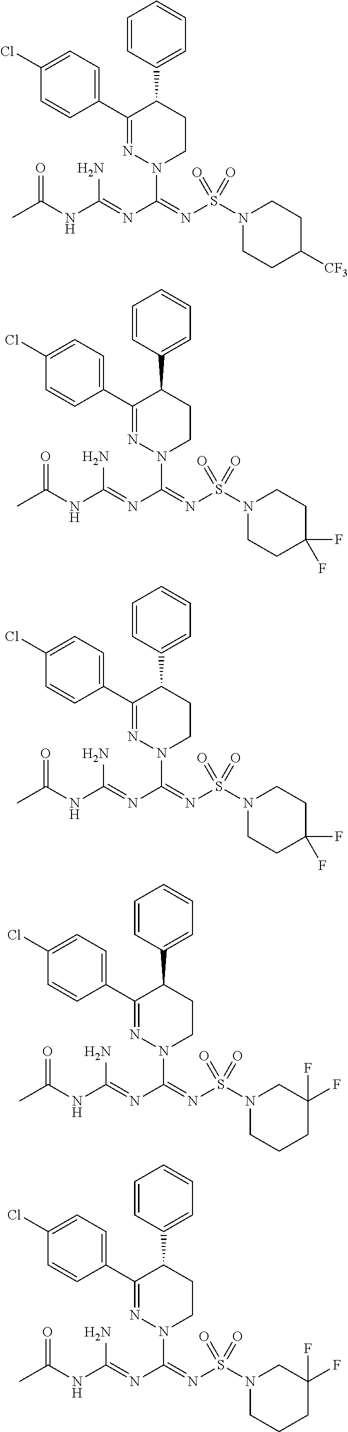 [SMILES: CC(=O)N/C(N)=N/C(=N/S(=O)(=O)N1CCC(C(F)(F)F)CC1)N1CC[C@@H](c2ccccc2)C(c2ccc(Cl)cc2)=N1 ; CC(=O)N/C(N)=N/C(=N/S(=O)(=O)N1CCC(F)(F)CC1)N1CC[C@H](c2ccccc2)C(c2ccc(Cl)cc2)=N1 ; CC(=O)N/C(N)=N/C(=N/S(=O)(=O)N1CCC(F)(F)CC1)N1CC[C@@H](c2ccccc2)C(c2ccc(Cl)cc2)=N1 ; CC(=O)N/C(N)=N/C(=N/S(=O)(=O)N1CCCC(F)(F)C1)N1CC[C@H](c2ccccc2)C(c2ccc(Cl)cc2)=N1 ; CC(=O)N/C(N)=N/C(=N/S(=O)(=O)N1CCCC(F)(F)C1)N1CC[C@@H](c2ccccc2)C(c2ccc(Cl)cc2)=N1]

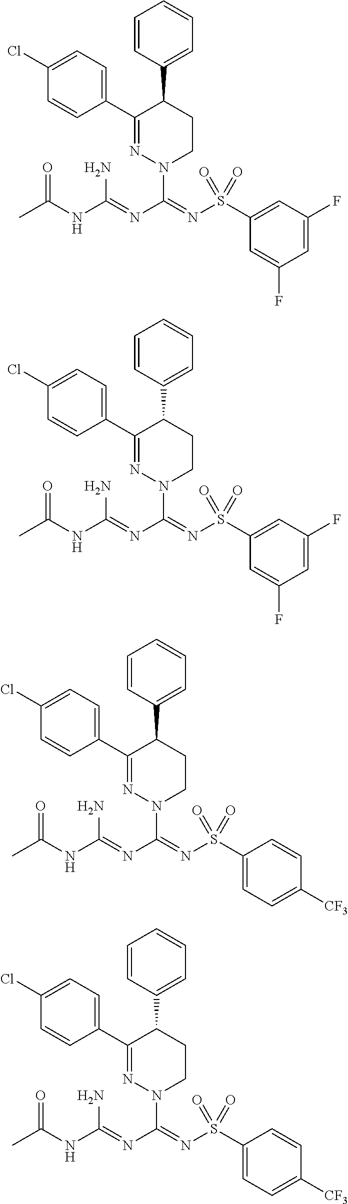 [SMILES: CC(=O)N/C(N)=N/C(=N/S(=O)(=O)c1cc(F)cc(F)c1)N1CC[C@H](c2ccccc2)C(c2ccc(Cl)cc2)=N1 ; CC(=O)N/C(N)=N/C(=N/S(=O)(=O)c1cc(F)cc(F)c1)N1CC[C@@H](c2ccccc2)C(c2ccc(Cl)cc2)=N1 ; CC(=O)N/C(N)=N/C(=N/S(=O)(=O)c1ccc(C(F)(F)F)cc1)N1CC[C@H](c2ccccc2)C(c2ccc(Cl)cc2)=N1 ; CC(=O)N/C(N)=N/C(=N/S(=O)(=O)c1ccc(C(F)(F)F)cc1)N1CC[C@@H](c2ccccc2)C(c2ccc(Cl)cc2)=N1]

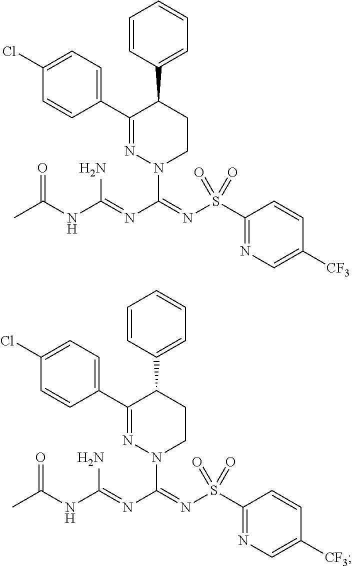 [SMILES: CC(=O)N/C(N)=N/C(=N/S(=O)(=O)c1ccc(C(F)(F)F)cn1)N1CC[C@H](c2ccccc2)C(c2ccc(Cl)cc2)=N1 ; CC(=O)N/C(N)=N/C(=N/S(=O)(=O)c1ccc(C(F)(F)F)cn1)N1CC[C@@H](c2ccccc2)C(c2ccc(Cl)cc2)=N1]

or a tautomer thereof, or a pharmaceutically acceptable salt thereof.
Embodiment 29. The compound of embodiment 2, wherein the compound is selected from:

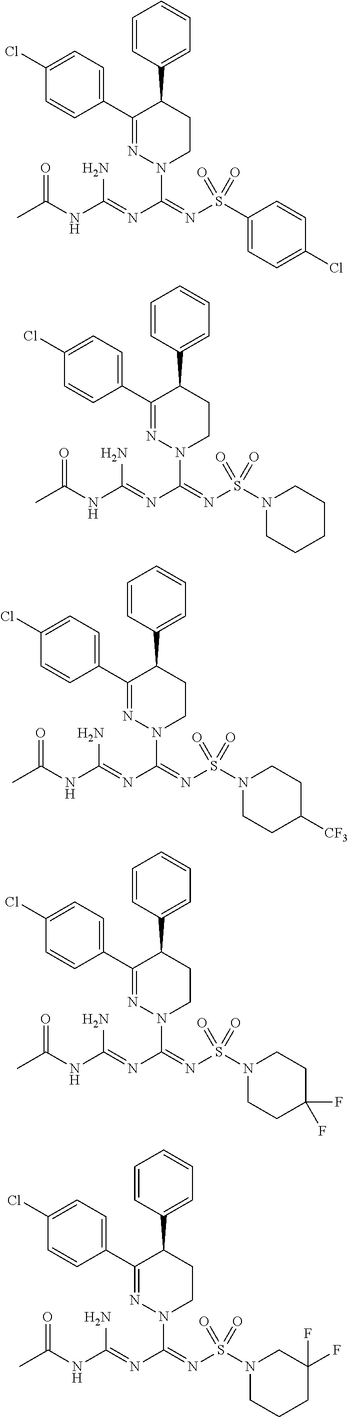 [SMILES: CC(=O)N/C(N)=N/C(=N/S(=O)(=O)c1ccc(Cl)cc1)N1CC[C@H](c2ccccc2)C(c2ccc(Cl)cc2)=N1 ; CC(=O)N/C(N)=N/C(=N/S(=O)(=O)N1CCCCC1)N1CC[C@H](c2ccccc2)C(c2ccc(Cl)cc2)=N1 ; CC(=O)N/C(N)=N/C(=N/S(=O)(=O)N1CCC(C(F)(F)F)CC1)N1CC[C@H](c2ccccc2)C(c2ccc(Cl)cc2)=N1 ; CC(=O)N/C(N)=N/C(=N/S(=O)(=O)N1CCC(F)(F)CC1)N1CC[C@H](c2ccccc2)C(c2ccc(Cl)cc2)=N1 ; CC(=O)N/C(N)=N/C(=N/S(=O)(=O)N1CCCC(F)(F)C1)N1CC[C@H](c2ccccc2)C(c2ccc(Cl)cc2)=N1]

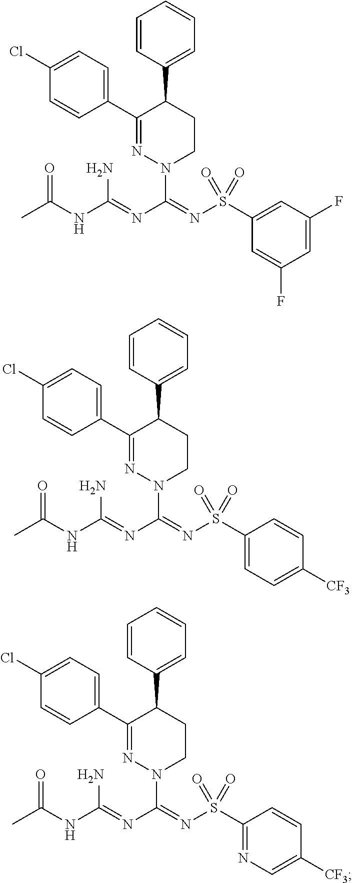 [SMILES: CC(=O)N/C(N)=N/C(=N/S(=O)(=O)c1cc(F)cc(F)c1)N1CC[C@H](c2ccccc2)C(c2ccc(Cl)cc2)=N1 ; CC(=O)N/C(N)=N/C(=N/S(=O)(=O)c1ccc(C(F)(F)F)cc1)N1CC[C@H](c2ccccc2)C(c2ccc(Cl)cc2)=N1 ; CC(=O)N/C(N)=N/C(=N/S(=O)(=O)c1ccc(C(F)(F)F)cn1)N1CC[C@H](c2ccccc2)C(c2ccc(Cl)cc2)=N1]

or a tautomer thereof, or a pharmaceutically acceptable salt thereof.
Embodiment 30. The compound of embodiment 3, wherein the compound is selected from:

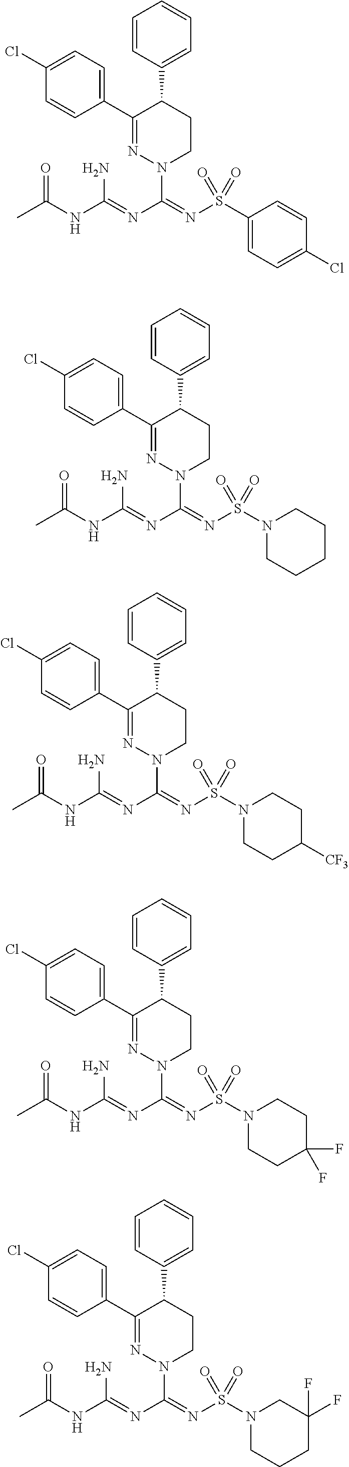 [SMILES: CC(=O)N/C(N)=N/C(=N/S(=O)(=O)c1ccc(Cl)cc1)N1CC[C@@H](c2ccccc2)C(c2ccc(Cl)cc2)=N1 ; CC(=O)N/C(N)=N/C(=N/S(=O)(=O)N1CCCCC1)N1CC[C@@H](c2ccccc2)C(c2ccc(Cl)cc2)=N1 ; CC(=O)N/C(N)=N/C(=N/S(=O)(=O)N1CCC(C(F)(F)F)CC1)N1CC[C@@H](c2ccccc2)C(c2ccc(Cl)cc2)=N1 ; CC(=O)N/C(N)=N/C(=N/S(=O)(=O)N1CCC(F)(F)CC1)N1CC[C@@H](c2ccccc2)C(c2ccc(Cl)cc2)=N1 ; CC(=O)N/C(N)=N/C(=N/S(=O)(=O)N1CCCC(F)(F)C1)N1CC[C@@H](c2ccccc2)C(c2ccc(Cl)cc2)=N1]

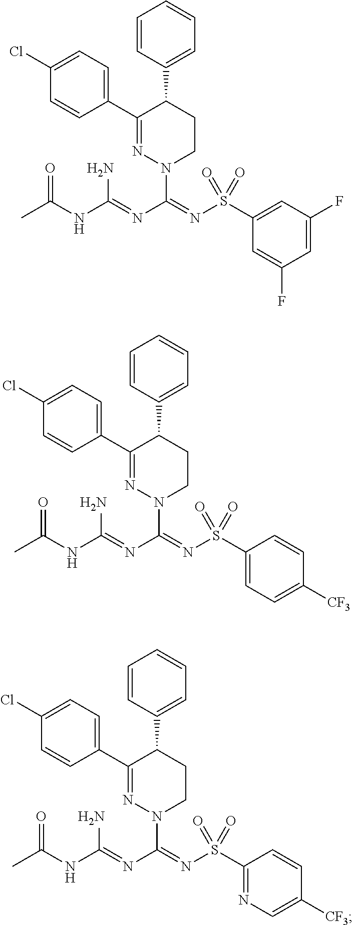 [SMILES: CC(=O)N/C(N)=N/C(=N/S(=O)(=O)c1cc(F)cc(F)c1)N1CC[C@@H](c2ccccc2)C(c2ccc(Cl)cc2)=N1 ; CC(=O)N/C(N)=N/C(=N/S(=O)(=O)c1ccc(C(F)(F)F)cc1)N1CC[C@@H](c2ccccc2)C(c2ccc(Cl)cc2)=N1 ; CC(=O)N/C(N)=N/C(=N/S(=O)(=O)c1ccc(C(F)(F)F)cn1)N1CC[C@@H](c2ccccc2)C(c2ccc(Cl)cc2)=N1]

or a tautomer thereof, or a pharmaceutically acceptable salt thereof.
Embodiment 31. A pharmaceutical composition comprising a compound as defined in any one of embodiments 1 to 30, together with a pharmaceutically acceptable carrier, diluent or excipient.
Embodiment 32. Use of a compound as defined in any one of embodiments 1 to 30 or a pharmaceutical composition as defined in embodiment 31 for the treatment of a disorder related to appetite or one of its complications, a disorder related to glucose regulation or one of its complications, a fibrosis related disorder or one of its complications, a disorder related to metabolism or one of its complications, a disorder related to skin and hair growth and healing, a disorder related to the GI tract, a disorder related to obesity or one of its complications, or a combination thereof.
Embodiment 33. The use of embodiment 32, wherein said disorder related to appetite or one of its complications is selected from Prader-Willi Syndrome (PWS), hypothalamic obesity, pro-opiomelanocortin (POMC) deficiency (including POMC obesity, heterozygous POMC deficiency obesity, POMC epigenetic disorders), leptin receptor (LepR) deficiency, Bardet-Biedl (BB) syndrome, and Alstr6m syndrome.
Embodiment 34. The use of embodiment 32, wherein said disorder related to glucose regulation or one of its complications is selected from diabetes Type I, diabetes Type II, insulin resistance, pre-diabetes, pancreatic diseases (by β-cell protection and/or increased insulin production), and associated nephropathies, neuropathies and retinopathies.
Embodiment 35. The use of embodiment 32, wherein said fibrosis related disorder or one of its complications is selected from progressive fibrosis associated with interstitial lung disease, idiopathic pulmonary fibrosis (IPF), Hermansky-Pudlak syndrome pulmonary fibrosis (HPS-PF), cirrhosis and other liver fibrosis disorders (such as nonalcoholic steatohepatitis (NASH), primary sclerosing cholangitis, primary biliary cholangitis), fibrotic renal diseases, skin fibrotic disorders (such as scleroderma, and chronic kidney diseases.
Embodiment 36. The use of embodiment 32, wherein said disorder related to metabolism or one of its complications is selected from metabolic syndrome and hyperlipidemia (e.g. hyper-triglyceridemia, hyper-triglyceridemia in the setting of low HDL-cholesterol, elevation of LDL and/or total cholesterol and/or VLDL and/or elevated Apolipoprotein B, atherosclerotic cardiovascular disease, etc.).
Embodiment 37. The use of embodiment 32, wherein said disorder related to obesity or one of its complications is selected from sleep apnea, snoring, asthma, pulmonary hypoventilation syndrome, dementia, heart disease, hypertension, gallbladder disease, gastrointestinal disorders, menstrual irregularities, degenerative arthritis, venous statis ulcer, coronary artery disease, arterial sclerotic disease, pseudotumor cerebri, osteoarthritis, high cholesterol, and increased incidence of malignancies of the liver, ovaries, cervix, uterus, breasts, prostate, or gallbladder.
Embodiment 38. The use of embodiment 32, wherein said disorder of the skin and hair is selected from alopecia (male pattern baldness and alopecia associated with metabolic syndrome), excessive scar formation (cicatrix and keloid), and scleroderma.
Embodiment 39. The use of embodiment 32, wherein said disorder related to the GI tract is selected from constipation, irritable bowel syndrome, and inflammatory bowel syndrome, including ulcerative colitis and Crohn's disease.
Embodiment 40. A method for the treatment of a disorder selected from disorders related to appetite or their complications, disorders related to glucose regulation or their complications, fibrosis related disorders or their complications, disorders related to metabolism or their complications, disorders related to skin and hair growth and healing, disorders related to the GI tract, disorders related to obesity or their complications, or a combination thereof, comprising administering a compound as defined in any one of embodiments 1 to 30 or a pharmaceutical composition as defined in embodiment 31 to a subject in need thereof.
Embodiment 41. The method of embodiment 40, wherein said disorders related to appetite or their complications are selected from Prader-Willi Syndrome (PWS), hypothalamic obesity, pro-opiomelanocortin (POMC) deficiency (including POMC obesity, heterozygous POMC deficiency obesity, POMC epigenetic disorders), leptin receptor (LepR) deficiency, Bardet-Biedl (BB) syndrome, and Alstr6m syndrome.
Embodiment 42. The method of embodiment 40, wherein said disorders related to glucose regulation or their complications are selected from diabetes Type I, diabetes Type II, insulin resistance, pre-diabetes, pancreatic diseases (by β-cell protection and/or increased insulin production), and associated nephropathies, neuropathies and retinopathies.
Embodiment 43. The method of embodiment 40, wherein said fibrosis related disorders or their complications are selected from progressive fibrosis associated with interstitial lung disease, idiopathic pulmonary fibrosis (IPF), Hermansky-Pudlak syndrome pulmonary fibrosis (HPS-PF), cirrhosis and other liver fibrosis disorders (such as nonalcoholic steatohepatitis (NASH), primary sclerosing cholangitis, primary biliary cholangitis), fibrotic renal diseases, skin fibrotic disorders (such as scleroderma, and chronic kidney diseases.
Embodiment 44. The method of embodiment 40, wherein said disorders related to metabolism or their complications are selected from metabolic syndrome and hyperlipidemia (e.g. hyper-triglyceridemia, hyper-triglyceridemia in the setting of low HDL-cholesterol, elevation of LDL and/or total cholesterol and/or VLDL and/or elevated Apolipoprotein B, atherosclerotic cardiovascular disease, etc.).
Embodiment 45. The method of embodiment 40, wherein said disorders related to obesity or their complications are selected from sleep apnea, snoring, obese asthma, pulmonary hypoventilation syndrome, dementia, heart disease, hypertension, gallbladder disease, gastrointestinal disorders, menstrual irregularities, degenerative arthritis, venous statis ulcer, coronary artery disease, arterial sclerotic disease, pseudotumor cerebri, osteoarthritis, high cholesterol, and increased incidence of malignancies of the liver, ovaries, cervix, uterus, breasts, prostate, or gallbladder.
Embodiment 46. The method of embodiment 40, wherein said disorders of the skin and hair is selected from alopecia (male pattern baldness and alopecia associated with metabolic syndrome), excessive scar formation (cicatrix and keloid), and scleroderma.
Embodiment 47. The method of embodiment 40, wherein said disorders related to the GI tract is selected from constipation, irritable bowel syndrome, and inflammatory bowel syndrome, including ulcerative colitis and Crohn's disease.

### DETAILED DESCRIPTION

All technical and scientific terms and expressions used herein have the same definitions as those commonly understood by a person skilled in the art to which the present technology pertains. The definition of some terms and expressions used is nevertheless provided below. To the extent the definitions of terms in the publications, patents, and patent applications incorporated herein by reference are contrary to the definitions set forth in this specification, the definitions in this specification will control. The section headings used herein are for organizational purposes only and are not to be construed as limiting the subject matter disclosed.

Chemical structures described herein are drawn according to conventional standards. Also, when an atom, such as a carbon atom, as drawn seems to include an incomplete valency, then the valency is assumed to be satisfied by one or more hydrogen atoms even though these are not necessarily explicitly drawn. Hydrogen atoms should be inferred to be part of the compound.

The terminology used herein is for the purpose of describing particular embodiments only and is not intended to be limiting. It should be noted that, the singular forms “a”, “an”, and “the” include plural forms as well, unless the content clearly dictates otherwise. Thus, for example, reference to a composition containing “a compound” also contemplates a mixture of two or more compounds. It should also be noted that the term “or” is generally employed in its sense including “and/or” unless the context clearly dictates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the terms “compounds”, “compounds herein described”, “compounds of the present application”, “tetrahydropyridazines”, “tetrahydropyridazine compounds” and equivalent expressions refer to compounds described in the present application, e.g. those encompassed by structural Formulae I, I(a), and I(b), optionally with reference to any of the applicable embodiments, and also includes exemplary compounds, such as Compounds 1 to 8, Compounds 1A to 8A, and Compounds 1B to 8B, their pharmaceutically acceptable salts and tautomeric forms, as well as solvates, esters, and prodrugs thereof when applicable. When a zwitterionic form is possible, the compound may be drawn as its neutral form for practical purposes, but the compound is understood to also include its zwitterionic form. Embodiments herein may also exclude one or more of the compounds. Compounds may be identified either by their chemical structure or their chemical name. In a case where the chemical structure and chemical name would conflict, the chemical structure will prevail.

Unless otherwise stated, structures depicted herein are also meant to include all isomeric (e.g., enantiomeric, diastereomeric, tautomeric, and geometric (or conformational)) forms of the structure when applicable; for example, the R and S configurations for each asymmetric center.

Therefore, single stereochemical isomers as well as enantiomeric, diastereomeric, tautomeric, and geometric (or conformational) mixtures of the present compounds are within the scope of the present description. The present compounds unless otherwise noted, also encompasses all possible tautomeric forms of the illustrated compound, if any. The term also includes isotopically labeled compounds where one or more atoms have an atomic mass different from the atomic mass most abundantly found in nature. Examples of isotopes that may be incorporated into the present compounds include, but are not limited to, ²H (D), ³H (T), ¹¹C, ¹³C, ¹⁴C, ¹⁵N, ¹⁸O, ¹⁷O, any one of the isotopes of sulfur, etc. The compounds may also exist in unsolvated forms as well as solvated forms, including hydrated forms. The compounds may exist in multiple crystalline or amorphous forms. In general, all physical forms are equivalent for the uses contemplated herein and are intended to be within the scope of the present invention.

Where a particular enantiomer is preferred, it may, in some embodiments be provided substantially free of the corresponding enantiomer and may also be enantiomerically enriched. “Enantiomerically enriched” means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including high-pressure liquid chromatography (HPLC) or supercritical Fluid Chromatography (SFC) on chiral support, or by the formation and crystallization of chiral salts or be prepared by asymmetric syntheses.

The expression “pharmaceutically acceptable salt” refers to those salts of the compounds of the present description which are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the present description, or separately by reacting a free base function of the compound with a suitable organic or inorganic acid (acid addition salts) or by reacting an acidic function of the compound with a suitable organic or inorganic base (base addition salts).

The term “solvate” refers to a physical association of one of the present compound with one or more solvent molecules, including water and non-aqueous solvent molecules. This physical association may include hydrogen bonding. In certain instances, the solvate will be capable of isolation, for example when one or more solvent molecules are incorporated in the crystal lattice of a crystalline solid. The term “solvate” encompasses both solution-phase and isolable solvates.

Exemplary solvates include, without limitation, hydrates, hemihydrates, ethanolates, hemiethanolates, n-propanolates, iso-propanolates, 1-butanolates, 2-butanolate, and solvates of other physiologically acceptable solvents, such as the Class 3 solvents described in the International Conference on Harmonization (ICH), Guide for Industry, Q3C Impurities: Residual Solvents (1997). Accordingly, the compound as herein described also includes each of its solvates and mixtures thereof.

As used herein, the expression “pharmaceutically acceptable ester” refers to esters of the compounds formed by the process of the present description which may hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates of hydroxyl groups, and alkyl esters of an acidic group. Other ester groups include sulfonate or sulfate esters.

The expression “pharmaceutically acceptable prodrugs” as used herein refers to those prodrugs of the compounds formed by the process of the present description which are, within the scope of sound medical judgment, suitable for use in contact with the tissues of humans and lower animals with undue toxicity, irritation, allergic response, and the like, commensurate with a reasonable benefit/risk ratio, and effective for their intended use. The term “prodrug”, as used herein, means a compound which is convertible in vivo by metabolic means (e.g. by hydrolysis) to afford any compound delineated by the formulae of the instant description.

Abbreviations may also be used throughout the application, unless otherwise noted, such abbreviations are intended to have the meaning generally understood by the field. Examples of such abbreviations include Me (methyl), Et (ethyl), Pr (propyl), i-Pr(isopropyl), Bu (butyl), t-Bu (tert-butyl), i-Bu (iso-butyl), s-Bu (sec-butyl), c-Bu (cyclobutyl), Ph (phenyl), Bn (benzyl), Bz (benzoyl), CBz or Cbz or Z (carbobenzyloxy), Boc or BOC (tert-butoxycarbonyl), and Su or Suc (succinimide).

The number of carbon atoms in a hydrocarbon substituent can be indicated by the prefix “Cₓ-C_{y}” or “C_{x-y}” where x is the minimum and y is the maximum number of carbon atoms in the substituent. However, when the prefix “Cₓ-C_{y}” or “C_{x-y}” is associated with a group incorporating one or more heteroatom(s) by definition (e.g. heterocycloalkyl, heteroaryl, etc.), then x and y define respectively the minimum and maximum number of atoms in the cycle, including carbon atoms as well as heteroatom(s).

The term “heteroatom” includes atoms other than carbon and hydrogen, such as, but not limited to nitrogen, oxygen, or sulfur, and includes any oxidized form of nitrogen or sulfur, substituted form of nitrogen, and any quaternized form of a basic nitrogen.

The term “alkyl” as used herein, refers to a saturated, straight- or branched-chain hydrocarbon radical typically containing from 1 to 20 carbon atoms. For example, “C₁₋₈alkyl” contains from one to eight carbon atoms. Examples of alkyl radicals include, but are not limited to, methyl, ethyl, propyl, isopropyl, n-butyl, tert-butyl, neopentyl, n-hexyl, heptyl, octyl radicals and the like.

The term “alkenyl” as used herein, denotes a straight- or branched-chain hydrocarbon radical containing one or more double bonds and typically from 2 to 20 carbon atoms. For example, “C₂₋₈alkenyl” contains from two to eight carbon atoms. Alkenyl groups include, but are not limited to, for example, ethenyl, propenyl, butenyl, 1-methyl-2-buten-1-yl, heptenyl, octenyl and the like.

The term “alkynyl” as used herein, denotes a straight- or branched-chain hydrocarbon radical containing one or more triple bonds and typically from 2 to 20 carbon atoms. For example, “C₂₋₈alkynyl” contains from two to eight carbon atoms. Representative alkynyl groups include, but are not limited to, for example, ethynyl,1-propynyl, 1-butynyl, heptynyl, octynyl and the like.

The terms “cycloalkyl”, “alicyclic”, “carbocyclic” and equivalent expressions refer to a group comprising a saturated or partially unsaturated (non-aromatic) carbocyclic ring in a monocyclic or polycyclic ring system, including spiro (sharing one atom), fused (sharing at least one bond) or bridged (sharing two or more bonds) carbocyclic ring systems, having from three to fifteen ring members. Examples of cycloalkyl groups include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, bicyclo[4,3,0]nonanyl, norbornyl, and the like. The term cycloalkyl includes both unsubstituted cycloalkyl groups and substituted cycloalkyl groups.

The term “C₃-ncycloalkyl” refers to a cycloalkyl group having from 3 to the indicated “n” number of carbon atoms in the ring structure. Unless the number of carbons is otherwise specified, “lower cycloalkyl” groups as herein used, have at least 3 and equal or less than 8 carbon atoms in their ring structure.

As used herein, the terms “heterocycloalkyl”, “heterocyclyl”, and the like are used interchangeably and refer to a chemically stable 3- to 7-membered monocyclic or 7-10-membered bicyclic heterocycloalkyl moiety that is either saturated or partially unsaturated, and having, in addition to carbon atoms, one or more, preferably one to four, heteroatoms, as defined above. As an example, in a saturated or partially unsaturated ring having 1-3 heteroatoms selected from oxygen, sulfur or nitrogen, the nitrogen may be N (as in 3,4-dihydro-2H-pyrrolyl), NH (as in pyrrolidinyl), or NR (as in N-substituted pyrrolidinyl). A heterocycloalkyl can be attached to its pendant group at any heteroatom or carbon atom that results in a chemically stable structure and any of the ring atoms can be optionally substituted. Examples of heterocycloalkyl groups include, but are not limited to, 1,3-dioxolanyl, pyrrolidinyl, pyrrolidonyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, tetrahydrofuranyl, tetrahydropyranyl, tetrahydrothiopyranyl, tetrahydrodithienyl, tetrahydrothienyl, thiomorpholino, thioxanyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, 2H-pyranyl, 4H-pyranyl, dioxanyl, dithianyl, dithiolanyl, dihydropyranyl, dihydrothienyl, dihydrofuranyl, 3-azabicyclo[3,1,0]hexanyl, 3-azabicyclo[4,1,0]heptanyl, quinolizinyl, quinuclidinyl, tetrahydroquinolinyl, tetrahydroisoquinolinyl, decahydroquinolinyl, and the like. Heterocycloalkyl groups also include groups in which a heterocycloalkyl ring is fused to one or more aryl, heteroaryl, or cycloaliphatic rings, such as indolinyl, 3H-indolyl, chromanyl, chromenyl, phenanthridinyl, 2-azabicyclo[2.2.1]heptanyl, octahydroindolyl, or tetrahydroquinolinyl, where the radical or point of attachment is on the heterocycloalkyl ring. A heterocycloalkyl group may be mono- or bicyclic. The term “heterocyclylalkyl” refers to an alkyl group substituted by a heterocycloalkyl, wherein the alkyl and heterocyclyl portions independently are optionally substituted. The term “C₃-nheterocycloalkyl” refers to a heterocycloalkyl group having from 3 to the indicated “n” number of atoms in the ring structure, including carbon atoms and heteroatoms.

As used herein, the term “partially unsaturated” refers to a ring moiety that includes at least one double or triple bond between ring atoms but is not aromatic. The term “partially unsaturated” is intended to encompass rings having multiple sites of unsaturation but is not intended to include aryl or heteroaryl moieties, as herein defined.

The term “aryl” used alone or as part of a larger moiety as in “aralkyl”, “aralkoxy”, “aryloxy”, or “aryloxyalkyl”, refers to aromatic groups having 4n+2 conjugated π(pi) electrons, wherein n is an integer from 1 to 3, in a monocyclic moiety or a bicyclic or tricyclic fused ring system having a total of six to 15 ring members, wherein at least one ring in the system is aromatic and wherein each ring in the system contains three to seven ring members. The term “aryl” may be used interchangeably with the expression “aryl ring”. In certain embodiments of the present description, “aryl” refers to an aromatic ring or ring system which includes, but is not limited to, phenyl, biphenyl, naphthyl, azulenyl, anthracyl and the like, which may bear one or more substituents.

The term “aralkyl” or “arylalkyl” refers to an alkyl residue attached to an aryl ring. Examples of aralkyl include, but are not limited to, benzyl, phenethyl, and the like. Also included within the scope of the term “aryl”, as it is used herein, is a group in which an aromatic ring is fused to one or more non-aromatic rings, such as indanyl, indenyl, phthalimidyl, naphthimidyl, fluorenyl, phenanthridinyl, or tetrahydronaphthyl, and the like. The term “C₆₋ₙaryl” refers to an aryl group having from 6 to the indicated “n” number of atoms in the ring structure.

The term “heteroaryl”, used alone or as part of a larger moiety, e.g., “heteroaralkyl”, or “heteroaralkoxy”, refers to aromatic groups having 4n+2 conjugated π(pi) electrons, wherein n is an integer from 1 to 3 (e.g. having 5 to 18 ring atoms, preferably 5, 6, or 9 ring atoms; having 6, 10, or 14 π electrons shared in a cyclic array); and having, in addition to carbon atoms, from one to five heteroatoms. The term “heteroatom” is as defined above. A heteroaryl may be a single ring, or two or more fused rings. The term “heteroaryl”, as used herein, also includes groups in which a heteroaromatic ring is fused to one or more aryl, cycloalkyl, or heterocycloalkyl rings. Nonlimiting examples of heteroaryl groups include thienyl, furanyl (furyl), pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, triazinyl, indolyl, 3H-indolyl, isoindolyl, indolizinyl, benzothienyl (benzothiophenyl), benzofuranyl, dibenzofuranyl, indazolyl, benzimidazolyl, benzoxazolyl, benzothiazolyl, benzotriazolyl, pyrrolopyridinyl (e.g. pyrrolo[3,2-b]pyridinyl or pyrrolo[3,2-c]pyridinyl), pyrazolopyridinyl (e.g. pyrazolo[1,5-a]pyridinyl), furopyridinyl, purinyl, imidazopyrazinyl (e.g. imidazo[4,5-b]pyrazinyl), quinolyl (quinolinyl), isoquinolyl (isoquinolinyl), quinolonyl, isoquinolonyl, cinnolinyl, phthalazinyl, quinazolinyl, quinoxalinyl, 4H-quinolizinyl, naphthyridinyl, and pteridinyl carbazolyl, acridinyl, phenanthridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. Heteroaryl groups include rings that are optionally substituted. The term “heteroaralkyl” refers to an alkyl group substituted by a heteroaryl, wherein the alkyl and heteroaryl portions are independently optionally substituted. Examples include, but are not limited to, pyridinylmethyl, pyrimidinylethyl and the like. For instance, the term “C₅-,heteroaryl” refers to a heteroaryl group having from 5 to the indicated “n” number of atoms in the ring structure, including carbon atoms and heteroatoms.

The term “halogen” or “halo” designates a halogen atom, i.e. a fluorine, chlorine, bromine or iodine atom, preferably fluorine or chlorine.

As described herein, compounds of the present description may contain “optionally substituted” moieties. In general, the term “substituted” means that one or more hydrogens of the designated moiety are replaced with a suitable substituent. Unless otherwise indicated, an “optionally substituted” group may have a suitable substituent at any or each substitutable position of the group, and when more than one position in any given structure may be substituted with more than one substituent selected from a specified group, the substituent may be either the same or different at each position. Combinations of substituents envisioned under the present description are preferably those that result in the formation of chemically stable or chemically feasible compounds. The term “chemically stable”, as used herein, refers to compounds that are not substantially altered when subjected to conditions to allow for their production, detection, and, in certain embodiments, their recovery, purification, and use for one or more of the purposes disclosed herein.

Examples of substituents include, but are not limited to halogen (F, C1, Br, 1), OH, CO₂H, alkoxy, oxo, thiooxo, NO₂, CN, CF₃, CHF₂, NH₂, NHalkyl, NHalkenyl, NHalkynyl, NHcycloalkyl, NHaryl, NHheteroaryl, NHheterocycloalkyl, dialkylamino, diarylamino, diheteroarylamino, O-alkyl, O-alkenyl, O-alkynyl, O-cycloalkyl, O-aryl, O-heteroaryl, O-haloalkyl, O-heterocycloalkyl, C(O)alkyl, C(O)alkenyl, C(O)alkynyl, C(O)cycloalkyl, C(O)aryl, C(O)heteroaryl, C(O)heterocycloalkyl, CO₂alkyl, CO₂alkenyl, CO₂alkynyl, CO₂cycloalkyl, CO₂aryl, CO₂heteroaryl, CO₂heterocycloalkyl, OC(O)alkyl, OC(O)alkenyl, OC(O)alkynyl, OC(O)cycloalkyl, OC(O)aryl, OC(O)heteroaryl, OC(O)heterocycloalkyl, C(O)NH₂, C(O)NHalkyl, C(O)NHalkenyl, C(O)NHalkynyl, C(O)NHcycloalkyl, C(O)NHaryl, C(O)NHheteroaryl, C(O)NHheterocycloalkyl, OCO₂alkyl, OCO₂alkenyl, OCO₂alkynyl, OCO₂cycloalkyl, OCO₂aryl, OCO₂heteroaryl, OCO₂heterocycloalkyl, OC(O)NH₂, OC(O)NHalkyl, OC(O)NHalkenyl, OC(O)NHalkynyl, OC(O)NHcycloalkyl, OC(O)NHaryl, OC(O)NHheteroaryl, OC(O)NHheterocycloalkyl, NHC(O)alkyl, NHC(O)alkenyl, NHC(O)alkynyl, NHC(O)cycloalkyl, NHC(O)aryl, NHC(O)heteroaryl, NHC(O)heterocycloalkyl, NHCO₂alkyl, NHCO₂alkenyl, NHCO₂alkynyl, NHCO₂cycloalkyl, NHCO₂aryl, NHCO₂heteroaryl, NHCO₂heterocycloalkyl, NHC(O)NH₂, NHC(O)NHalkyl, NHC(O)NHalkenyl, NHC(O)NHalkenyl, NHC(O)NHcycloalkyl, NHC(O)NHaryl, NHC(O)NHheteroaryl, NHC(O)NHheterocycloalkyl, NHC(S)NH₂, NHC(S)NHalkyl, NHC(S)NHalkenyl, NHC(S)NHalkynyl, NHC(S)NHcycloalkyl, NHC(S)NHaryl, NHC(S)NHheteroaryl, NHC(S)NHheterocycloalkyl, NHC(NH)NH₂, NHC(NH)NHalkyl, NHC(NH)NHalkenyl, NHC(NH)NHalkenyl, NHC(NH)NHcycloalkyl, NHC(NH)NHaryl, NHC(NH)NHheteroaryl, NHC(NH)NHheterocycloalkyl, NHC(NH)alkyl, NHC(NH)alkenyl, NHC(NH)alkenyl, NHC(NH)cycloalkyl, NHC(NH)aryl, NHC(NH)heteroaryl, NHC(NH)heterocycloalkyl, C(NH)NHalkyl, C(NH)NHalkenyl, C(NH)NHalkynyl, C(NH)NHcycloalkyl, C(NH)NHaryl, C(NH)NHheteroaryl, C(NH)NHheterocycloalkyl, S(O)alkyl, S(O)alkenyl, S(O)alkynyl, S(O)cycloalkyl, S(O)aryl, S(O)₂alkyl, S(O)₂alkenyl, S(O)₂alkynyl, S(O)₂cycloalkyl, S(O)₂aryl, S(O)heteroaryl, S(O)heterocycloalkyl, SO₂NH₂, SO₂NHalkyl, SO₂NHalkenyl, SO₂NHalkynyl, SO₂NHcycloalkyl, SO₂NHaryl, SO₂NHheteroaryl, SO₂NHheterocycloalkyl, NHSO₂alkyl, NHSO₂alkenyl, NHSO₂alkynyl, NHSO₂cycloalkyl, NHSO₂aryl, NHSO₂heteroaryl, NHSO₂heterocycloalkyl, CH₂NH₂, CH₂SO₂CH₃, alkyl, alkenyl, alkynyl, aryl, arylalkyl, heteroaryl, heteroarylalkyl, heterocycloalkyl, cycloalkyl, carbocyclic, heterocycloalkyl, polyalkoxyalkyl, polyalkoxy, methoxymethoxy, methoxyethoxy, SH, S-alkyl, S-alkenyl, S-alkynyl, S-cycloalkyl, S-aryl, S-heteroaryl, S-heterocycloalkyl, or methylthiomethyl. Each of these substituents may also be further substituted where possible.

The present document therefore to tetrahydropyridazine compounds as defined herein and in the following paragraphs. When referring to chemical moieties, the recitation of a listing of chemical groups in any definition of a variable includes definitions of that variable as any single group or combination of listed groups. Similarly, the recitation of an embodiment for a variable herein includes that embodiment as any single embodiment or in combination with any other embodiments or portions thereof. As such, the following embodiments are present alone or in combination if applicable.

The present compounds present a tetrahydropyridazine core structure to which is attached defined substituents. Exemplary compounds as defined herein are illustrated by the general Formula I:

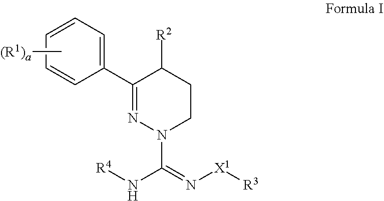

wherein,
R¹ is independently in each occurrence selected from optionally substituted alkyl, optionally substituted cycloalkyl, optionally substituted heterocycloalkyl, halogen, cyano, nitro, hydroxy, optionally substituted alkoxy, amino, optionally substituted alkylsulfonyl, optionally substituted aryl, optionally substituted heteroaryl, optionally substituted carboxyl, acyl, optionally substituted alkenyl, optionally substituted alkynyl, optionally substituted phosphonyl, optionally substituted phosphinyl, optionally substituted boronate, optionally substituted silyl, and imino;
R² and R³ are each independently selected from optionally substituted alkyl, aryl, heteroaryl, cycloalkyl, and heterocycloalkyl groups;
R⁴ is selected from —C(O)R⁵, —S(O)₂R⁵, —C(═NR⁶)R⁵, and —C(═NR⁶)NHC(O)R⁵;
R⁵ is selected from optionally substituted alkyl, optionally substituted alkoxy, optionally substituted alkylamino or dialkylamino, optionally substituted cycloalkyl, optionally substituted heterocycloalkyl, optionally substituted aryl, and optionally substituted heteroaryl;
R⁶ is selected from hydrogen, optionally substituted alkyl, optionally substituted alkoxy, optionally substituted alkylamino or dialkylamino, optionally substituted cycloalkyl, optionally substituted heterocycloalkyl, optionally substituted aryl, and optionally substituted heteroaryl, or R⁶ is taken together with R⁵ and their adjacent atoms to form an optionally substituted heterocycloalkyl or heteroaryl;
X¹ is selected from SO₂ and C═O; and
a is 0, 1, 2, 3, 4 or 5;
or an isomer and/or tautomer thereof, or a pharmaceutically acceptable salt thereof.

In the above formula, the compounds may be enriched in a particular isomer, for instance of Formula I(a):

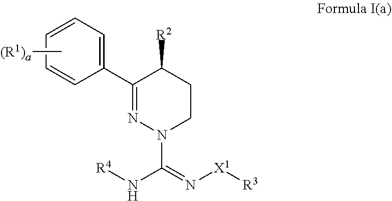

wherein R¹ to R⁴, X¹, and a are as previously defined, or an isomer and/or tautomer thereof, or a pharmaceutically acceptable salt thereof.

In the above formula, the compounds may be enriched is a different isomer, for instance an isomer of Formula I(b):

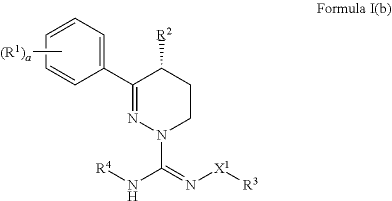

wherein R¹ to R⁴, X¹, and a are as previously defined, or an isomer and/or tautomer thereof, or a pharmaceutically acceptable salt thereof.

In some examples of the above formulae, R⁴ is —C(O)R⁵, —C(═NR⁶)R⁵, or —C(═NR⁶)NHC(O)R⁵, preferably R⁴ is —C(═NR⁶)NHC(O)R⁵.

R⁵ may be defined as an optionally substituted C₁₋₆alkyl, optionally substituted C₁₋₆alkoxy, or an optionally substituted C₁₋₆alkylamino or di C₁₋₆alkylamino group, for example R⁵ may be a C₁₋₆alkyl group, such as methyl, ethyl, propyl, etc. (for instance a methyl group).

R⁵ may also be defined as an optionally substituted cycloalkyl, optionally substituted heterocycloalkyl, optionally substituted aryl, or optionally substituted heteroaryl group.

In some compounds, R⁶ is H. In other compounds, R⁶ can be taken together with R⁵ and their adjacent atoms to form an optionally substituted heterocycloalkyl or heteroaryl.

In some examples, X¹ may be C═O. In other examples, X¹ is SO₂.

For example, in the compounds of Formulae I, I(a) and I(b), R¹ is halogen atom, for example R¹ is a chlorine atom.

Some of the compounds of Formulae I, I(a) and I(b) may include an R³ group being an optionally substituted C₂₋₆alkyl group. In other compounds, R³ may be an optionally substituted C₆aryl, C₅₋₆ heteroaryl or C₅₋₇heterocycloalkyl group.

For example, R³ may be a functional group of the formula:

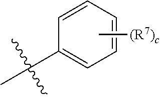

wherein,
R⁷ is independently in each occurrence selected from halogen, OH, CN, NO₂, C(O)R⁹, C(O)N(R⁸)₂, C(R¹⁰)═NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, OC₁₋₆alkyl, C₅₋₁₀aryl, C₅₋₁₀heteroaryl, C₃₋₁₀cycloalkyl, and C₄₋₁₀heterocycloalkyl;
R⁸ is independently in each occurrence selected from H, C₁₋₆alkyl, C₂₋₆alkenyl, C₂₋₆alkynyl, C₃₋₁₀cycloalkyl, C₄₋₁₀heterocycloalkyl, C₆aryl, and C₅₋₁₀heteroaryl, or two R⁸ are taken together with their adjacent atom(s) to form a C₄₋₁₀heterocycloalkyl group;
R⁹ is independently in each occurrence selected from C₁₋₆alkyl, C₂₋₆alkenyl, C₂₋₆alkynyl, C₃₋₇cycloalkyl, C₆aryl, and C₅₋₆heteroaryl; and
R¹⁰ is independently in each occurrence selected from H, C₁₋₆alkyl, C₂₋₆alkenyl, C₂₋₆alkynyl, C₃₋₇-cycloalkyl, C₆aryl, and C₅₋₆heteroaryl; and
c is 0,1,2,3,4 or 5;
wherein said alkyl, alkenyl, alkynyl, cycloalkyl, heterocycloalkyl, aryl or heteroaryl group is optionally further substituted.

Alternatively, R³ may be a functional group of the formula:

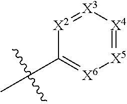

wherein,
X², X³, X⁴, X⁵, and X⁶ are each independently selected from N and CR¹¹, wherein at most three of X², X³, X⁴, X⁵, and X⁶ are N.
R¹¹ is independently in each occurrence selected from hydrogen, halogen, OH, CN, NO₂, C(O)R⁹, C(O)N(R⁸)₂, C(R¹⁰)═NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, OC₁₋₆alkyl, C₆-10aryl, C₅₋₁₀heteroaryl, C₃₋₁₀cycloalkyl, and C₄₋₁₀heterocycloalkyl; and
c is 0, 1, 2, 3, 4 or 5;
wherein R⁸, R⁹, and R¹⁰ are as defined herein; and
wherein said alkyl, alkenyl, alkynyl, cycloalkyl, heterocycloalkyl, aryl or heteroaryl group is optionally further substituted.

On the other hand, R³ may rather be a functional group of the formula:

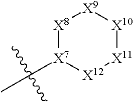

wherein,
X⁷ is selected from N and CR¹¹;
X⁸, X⁹, X¹⁰, X¹¹, and X¹² are each independently selected from O, NR^{x} and C(R^{y})₂, wherein at most two of X⁷, X⁸, X⁹, X¹⁰, X¹¹, and X¹² are or comprise 0 or N;
R^{x} is selected from is independently in each occurrence selected from hydrogen, C(O)R⁹, C(O)N(R⁸)₂, SO₂R⁹, SO₂N(R⁸)₂, P(O)(R⁸)₂, P(O)(OR⁸)₂, B(OR⁸)₂, C₁₋₆alkyl, C₂₋₆alkenyl, C₂₋₆alkynyl, C₆-10aryl, C₅₋₁₀heteroaryl, C₃₋₁₀cycloalkyl, and C₄₋₁₀heterocycloalkyl;
R^{y} is independently in each occurrence selected from hydrogen, halogen, OH, CN, NO₂, C(O)R⁹, C(O)N(R⁸)₂, C(R¹⁰)═NR¹⁰, SO₂R⁹, SO₂N(R⁸)₂, N(R¹⁰)C(O)R⁹, N(R¹⁰)SO₂R⁹, N(R¹⁰)C(O)N(R⁸)₂, N(R¹⁰)SO₂N(R⁸)₂, N(R⁸)₂, P(O)(R⁸)₂, P(O)(OR⁸)₂, B(OR⁸)₂, C₁₋₆alkyl, C₂₋₆alkenyl, C₂₋₆alkynyl, OC₁₋₆alkyl, C₆-10aryl, C₅₋₁₀heteroaryl, C₃₋₁₀cycloalkyl, and C₄₋₁₀heterocycloalkyl or two R^{y} are taken together with their adjacent atom(s) to form a C₄₋₁₀cycloalkyl or C₄₋₁₀heterocycloalkyl group;
wherein R⁸, R⁹, R¹⁰ and R¹¹ is as defined herein; and
wherein said alkyl, alkenyl, alkynyl, cycloalkyl, heterocycloalkyl, aryl or heteroaryl group is optionally further substituted.

In some examples, R¹¹ or R^{y} is a halogen (e.g. F or CI) or halogenated C₁₋₆alkyl (e.g. fluorinated C₁-3alkyl), in at least one occurrence. In other examples, R¹¹ or R^{y} is a hydrogen atom in each occurrence.

Some of the compounds of Formulae 1, I(a) and I(b) may include an R² group being an optionally substituted C₂₋₆alkyl group. In other compounds, R² may be an optionally substituted C₆aryl, C₅₋₆heteroaryl or C₅₋₇heterocycloalkyl group.

For example, R² may be a functional group of the formula:

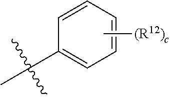

wherein,
R¹² is independently in each occurrence selected from halogen, OH, CN, NO₂, C(O)R¹⁴, C(O)N(R¹³)₂, C(R¹⁵)═NR¹⁵, SO₂R¹⁴, SO₂N(R¹³)₂, N(R¹⁵)C(O)R¹⁴, N(R¹⁵)SO₂R¹⁴, N(R¹⁵)C(O)N(R¹³)₂, N(R¹⁵)SO₂N(R¹³)₂, N(R¹³)₂, P(O)(R¹³)₂, P(O)(OR¹³)₂, B(OR¹³)₂, C₁₋₆alkyl, C₂₋₆alkenyl, C₂₋₆alkynyl, OC₁₋₆alkyl, C₆-10aryl, C₅₋₁₀heteroaryl, C₃₋₁₀cycloalkyl, and C₄₋₁₀heterocycloalkyl;
R¹³ is independently in each occurrence selected from H, C₁₋₆alkyl, C₂₋₆alkenyl, C₂₋₆alkynyl, C₃₋₁₀cycloalkyl, C₄₋₁₀heterocycloalkyl, C₆aryl, and C₅₋₁₀heteroaryl, or two R⁸ are taken together with their adjacent atom(s) to form a C₄₋₁₀heterocycloalkyl group;
R¹⁴ is independently in each occurrence selected from C₁₋₆alkyl, C₂₋₆alkenyl, C₂₋₆alkynyl, C₃₋₇cycloalkyl, C₆aryl, and C₅₋₆heteroaryl; and
R¹⁵ is independently in each occurrence selected from H, C₁₋₆alkyl, C₂₋₆alkenyl, C₂₋₆alkynyl, C₃₋₇-cycloalkyl, C₆aryl, and C₅₋₆heteroaryl; and
c is 0, 1, 2, 3, 4 or 5;
wherein said alkyl, alkenyl, alkynyl, cycloalkyl, heterocycloalkyl, aryl or heteroaryl group is optionally further substituted.

In the alternative, R² may be a functional group of the formula:

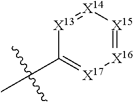

wherein,
X¹³, X¹⁴, X¹⁵, X¹⁶, and X¹⁷ are each independently selected from N and CR¹⁶, wherein at most three of X¹³, X¹⁴, X¹⁵, X¹⁶, and X¹⁷ are N.

R¹⁶ is independently in each occurrence selected from hydrogen, halogen, OH, CN, NO₂, C(O)R¹⁴, C(O)N(R¹³)₂, C(R¹⁵)═NR¹⁵, SO₂R¹⁴, SO₂N(R¹³)₂, N(R¹⁵)C(O)R¹⁴, N(R¹⁵)SO₂R¹⁴, N(R¹⁵)C(O)N(R¹³)₂, N(R¹⁵)SO₂N(R¹³)₂, N(R¹³)₂, P(O)(R¹³)₂, P(O)(OR¹³)₂, B(OR¹³)₂, C₁₋₆alkyl, C₂₋₆alkenyl, C₂₋₆alkynyl, OC₁₋₆alkyl, C₆-10aryl, C₅₋₁₀heteroaryl, C₃₋₁₀cycloalkyl, and C₄₋₁₀heterocycloalkyl; and
c is 0,1,2,3,4 or 5;
wherein R¹³, R¹⁴, and R¹⁵ are as defined herein; and
wherein said alkyl, alkenyl, alkynyl, cycloalkyl, heterocycloalkyl, aryl or heteroaryl group is optionally further substituted.

Or, R² may be a functional group of the formula:

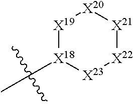

wherein,
X¹⁸ is selected from N and CR¹⁶;
X¹⁹, X²⁰, X²¹, X²², and X²³ are each independently selected from O, NR^{w} and C(R^{z})₂, wherein at most two of X¹⁸, X¹⁹, X²⁰, X²¹, X²², and X²³ are or comprise 0 or N;
R^{w} is selected from is independently in each occurrence selected from hydrogen, C(O)R¹⁴, C(O)N(R¹³)₂, SO₂R¹⁴, SO₂N(R¹³)₂, P(O)(R¹³)₂, P(O)(OR¹³)₂, B(OR¹³)₂, C₁₋₆alkyl, C₂₋₆alkenyl, C₂₋₆alkynyl, C₆-10aryl, C₅₋₁₀heteroaryl, C₃₋₁₀cycloalkyl, and C₄₋₁₀heterocycloalkyl;
R^{z} is independently in each occurrence selected from hydrogen, halogen, OH, CN, NO₂, C(O)R¹⁴, C(O)N(R¹³)₂, C(R¹⁵)═NR¹⁵, SO₂R¹⁴, SO₂N(R¹³)₂, N(R¹⁵)C(O)R¹⁴, N(R¹⁵)SO₂R¹⁴, N(R¹⁵)C(O)N(R¹³)₂, N(R¹⁵)SO₂N(R¹³)₂, N(R¹³)₂, P(O)(R¹³)₂, P(O)(OR¹³)₂, B(OR¹³)₂, C₁₋₆alkyl, C₂₋₆alkenyl, C₂₋₆alkynyl, OC₁₋₆alkyl, C₆-10aryl, C₅₋₁₀heteroaryl, C₃₋₁₀cycloalkyl, and C₄₋₁₀heterocycloalkyl or two R^{z} are taken together with their adjacent atom(s) to form a C₄₋₁₀cycloalkyl or C₄₋₁₀heterocycloalkyl group;
wherein R¹³, R¹⁴, R¹⁵ and R¹⁶ are is as defined herein; and
wherein said alkyl, alkenyl, alkynyl, cycloalkyl, heterocycloalkyl, aryl or heteroaryl group is optionally further substituted.

In some examples, R¹⁶ or R^{z} is a hydrogen atom in each occurrence. In other examples, R¹⁶ or R^{z} is a halogen (e.g. F or CI) or halogenated C₁₋₆alkyl (e.g. fluorinated C₁-3alkyl), in at least one occurrence.

Non-limiting examples of the compounds of Formula I comprise:

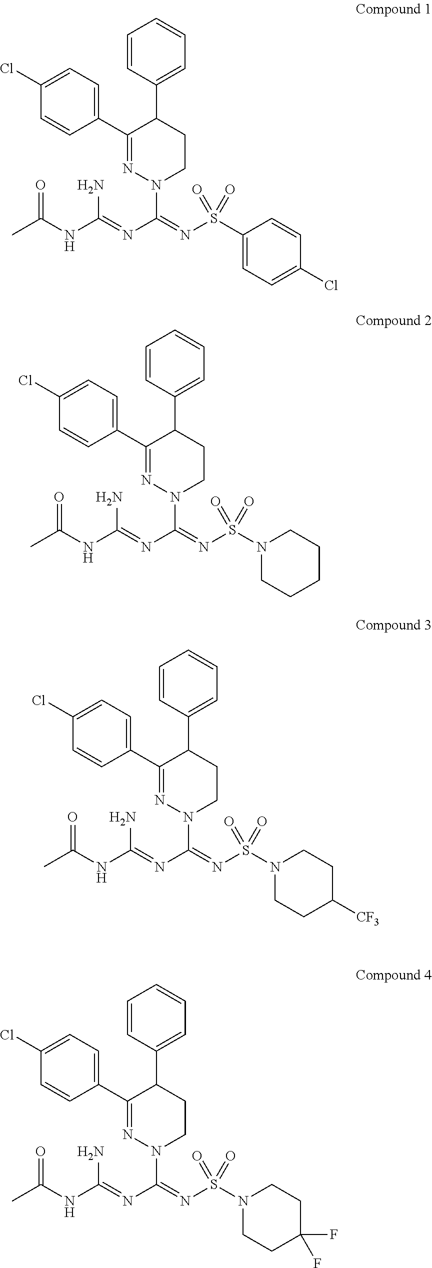 [SMILES: CC(=O)N/C(N)=N/C(=N/S(=O)(=O)c1ccc(Cl)cc1)N1CCC(c2ccccc2)C(c2ccc(Cl)cc2)=N1 ; CC(=O)N/C(N)=N/C(=N/S(=O)(=O)N1CCCCC1)N1CCC(c2ccccc2)C(c2ccc(Cl)cc2)=N1 ; CC(=O)N/C(N)=N/C(=N/S(=O)(=O)N1CCC(C(F)(F)F)CC1)N1CCC(c2ccccc2)C(c2ccc(Cl)cc2)=N1 ; CC(=O)N/C(N)=N/C(=N/S(=O)(=O)N1CCC(F)(F)CC1)N1CCC(c2ccccc2)C(c2ccc(Cl)cc2)=N1]

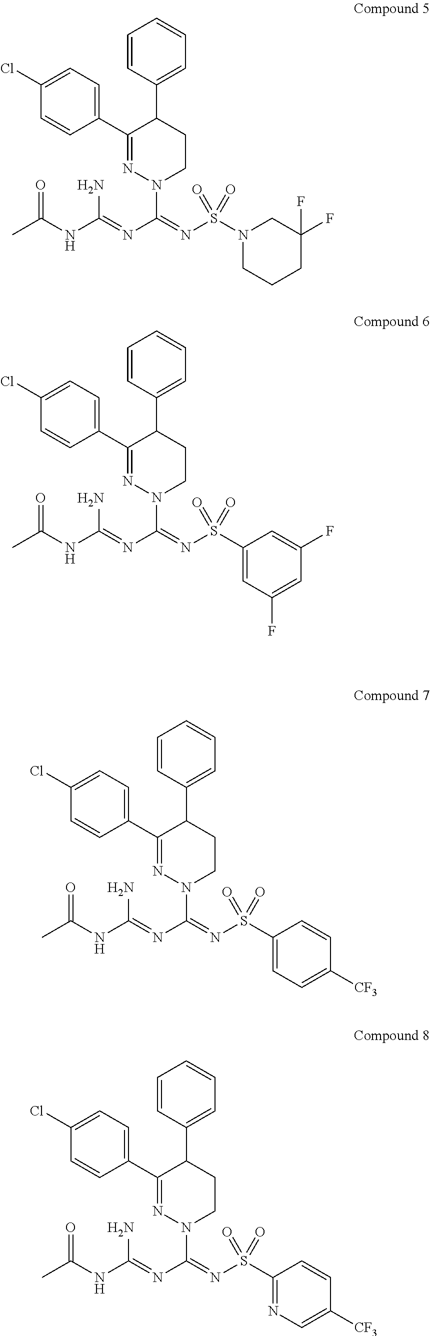 [SMILES: CC(=O)N/C(N)=N/C(=N/S(=O)(=O)N1CCCC(F)(F)C1)N1CCC(c2ccccc2)C(c2ccc(Cl)cc2)=N1 ; CC(=O)N/C(N)=N/C(=N/S(=O)(=O)c1cc(F)cc(F)c1)N1CCC(c2ccccc2)C(c2ccc(Cl)cc2)=N1 ; CC(=O)N/C(N)=N/C(=N/S(=O)(=O)c1ccc(C(F)(F)F)cc1)N1CCC(c2ccccc2)C(c2ccc(Cl)cc2)=N1 ; CC(=O)N/C(N)=N/C(=N/S(=O)(=O)c1ccc(C(F)(F)F)cn1)N1CCC(c2ccccc2)C(c2ccc(Cl)cc2)=N1]

or an isomer and/or tautomer thereof, or a pharmaceutically acceptable salt thereof.

Non-limiting examples of the compounds of Formula I(a) comprise:

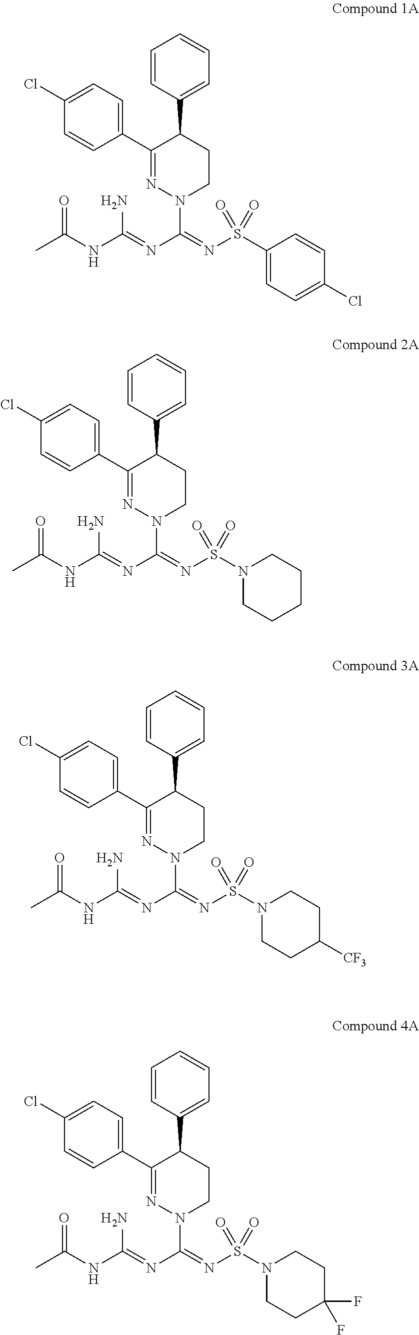 [SMILES: CC(=O)N/C(N)=N/C(=N/S(=O)(=O)c1ccc(Cl)cc1)N1CC[C@H](c2ccccc2)C(c2ccc(Cl)cc2)=N1 ; CC(=O)N/C(N)=N/C(=N/S(=O)(=O)N1CCCCC1)N1CC[C@H](c2ccccc2)C(c2ccc(Cl)cc2)=N1 ; CC(=O)N/C(N)=N/C(=N/S(=O)(=O)N1CCC(C(F)(F)F)CC1)N1CC[C@H](c2ccccc2)C(c2ccc(Cl)cc2)=N1 ; CC(=O)N/C(N)=N/C(=N/S(=O)(=O)N1CCC(F)(F)CC1)N1CC[C@H](c2ccccc2)C(c2ccc(Cl)cc2)=N1]

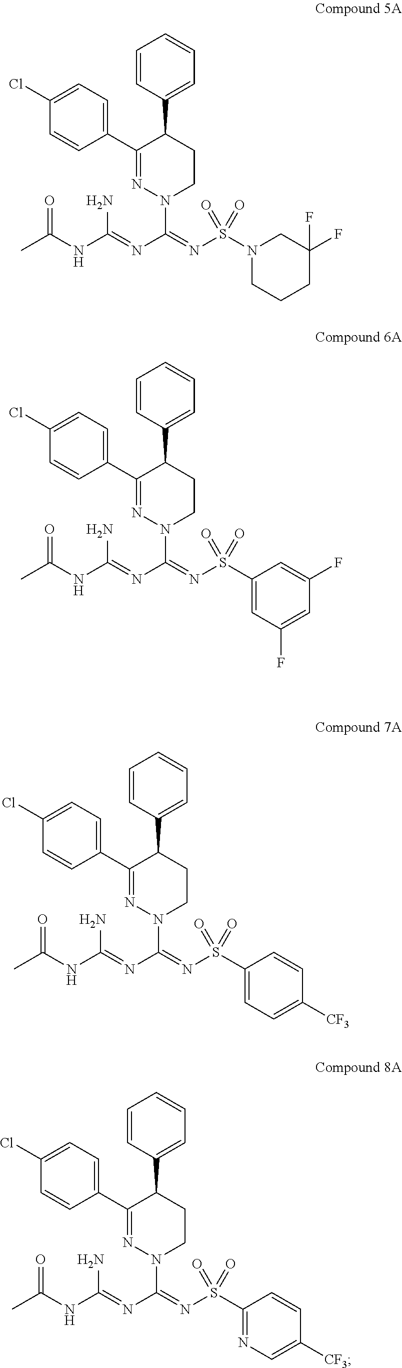 [SMILES: CC(=O)N/C(N)=N/C(=N/S(=O)(=O)N1CCCC(F)(F)C1)N1CC[C@H](c2ccccc2)C(c2ccc(Cl)cc2)=N1 ; CC(=O)N/C(N)=N/C(=N/S(=O)(=O)c1cc(F)cc(F)c1)N1CC[C@H](c2ccccc2)C(c2ccc(Cl)cc2)=N1 ; CC(=O)N/C(N)=N/C(=N/S(=O)(=O)c1ccc(C(F)(F)F)cc1)N1CC[C@H](c2ccccc2)C(c2ccc(Cl)cc2)=N1 ; CC(=O)N/C(N)=N/C(=N/S(=O)(=O)c1ccc(C(F)(F)F)cn1)N1CC[C@H](c2ccccc2)C(c2ccc(Cl)cc2)=N1]

or a tautomer thereof, or a pharmaceutically acceptable salt thereof.

Non-limiting examples of the compounds of Formula I(b) comprise:

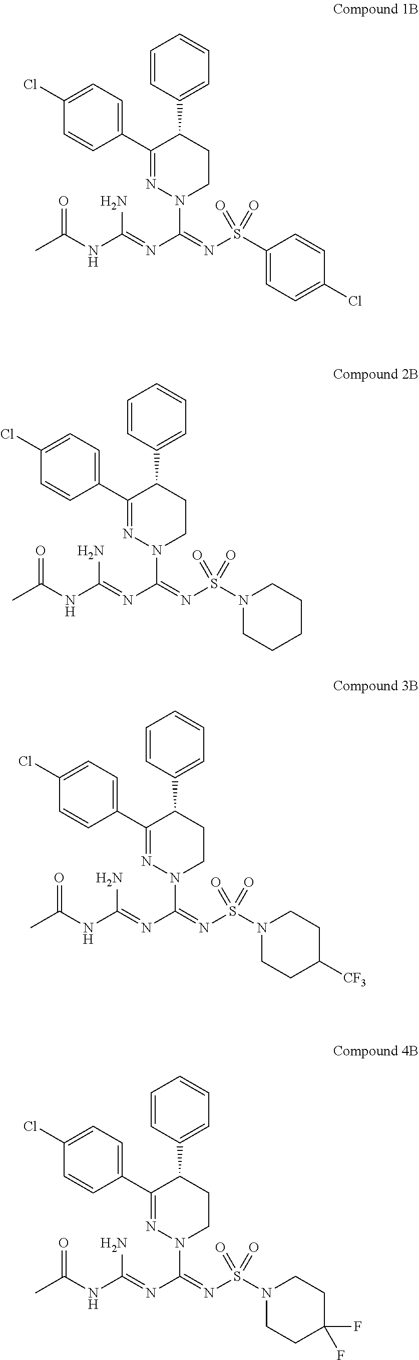 [SMILES: CC(=O)N/C(N)=N/C(=N/S(=O)(=O)c1ccc(Cl)cc1)N1CC[C@@H](c2ccccc2)C(c2ccc(Cl)cc2)=N1 ; CC(=O)N/C(N)=N/C(=N/S(=O)(=O)N1CCCCC1)N1CC[C@@H](c2ccccc2)C(c2ccc(Cl)cc2)=N1 ; CC(=O)N/C(N)=N/C(=N/S(=O)(=O)N1CCC(C(F)(F)F)CC1)N1CC[C@@H](c2ccccc2)C(c2ccc(Cl)cc2)=N1 ; CC(=O)N/C(N)=N/C(=N/S(=O)(=O)N1CCC(F)(F)CC1)N1CC[C@@H](c2ccccc2)C(c2ccc(Cl)cc2)=N1]

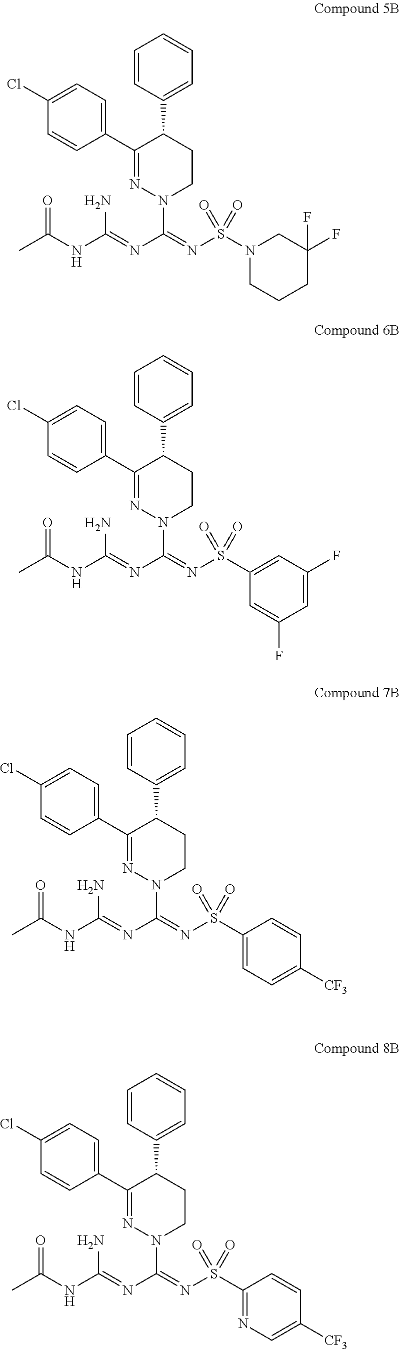 [SMILES: CC(=O)N/C(N)=N/C(=N/S(=O)(=O)N1CCCC(F)(F)C1)N1CC[C@@H](c2ccccc2)C(c2ccc(Cl)cc2)=N1 ; CC(=O)N/C(N)=N/C(=N/S(=O)(=O)c1cc(F)cc(F)c1)N1CC[C@@H](c2ccccc2)C(c2ccc(Cl)cc2)=N1 ; CC(=O)N/C(N)=N/C(=N/S(=O)(=O)c1ccc(C(F)(F)F)cc1)N1CC[C@@H](c2ccccc2)C(c2ccc(Cl)cc2)=N1 ; CC(=O)N/C(N)=N/C(=N/S(=O)(=O)c1ccc(C(F)(F)F)cn1)N1CC[C@@H](c2ccccc2)C(c2ccc(Cl)cc2)=N1]

or a tautomer thereof, or a pharmaceutically acceptable salt thereof.

These compounds may be prepared by conventional chemical synthesis such as those described in the Examples section below. As can be appreciated by the skilled artisan, further methods of synthesizing the compounds of the formulae herein will be evident to those of ordinary skill in the art. Additionally, the various synthetic steps may be performed in an alternate sequence or order to give the desired compounds.

The compound as defined herein can be formulated in a pharmaceutical composition for administration to a subject, the compound being usually admixed with a at least one pharmaceutically acceptable carrier, diluent, or excipient.

The expression “pharmaceutically acceptable carrier, diluent, or excipient” and equivalent expressions, refer to a non-toxic carrier, diluent, or excipient that does not destroy the pharmacological activity of the compound with which it is formulated.

Compositions described herein may be administered orally, parenterally, by inhalation spray, topically, rectally, nasally, buccally, or via an implanted reservoir. The term “parenteral” as used herein includes subcutaneous, intravenous, intramuscular, intraarticular, intrasynovial, intrasternal, intrathecal, intrahepatic, and intralesional injection or infusion techniques. Other modes of administration also include intradermal or transdermal administration.

For instance, solid dosage forms for oral administration include capsules, tablets, pills, and granules. In a preferred alternative, the composition is a solid dosage form which comprises the compound as described herein and at least one binder as defined in the preceding paragraph, the binder preferably comprising microcrystalline cellulose.

Pharmaceutically acceptable carriers, diluents or excipients that may be used in oral compositions of this disclosure include, but are not limited to, binders, sweeteners, disintegrating agents, diluents, flavorings, coating agents, preservatives, lubricants, and/or polymers. Examples of binders include cellulose-based substances such as microcrystalline cellulose and carboxymethylcellulose, and other binders like gum acacia, gelatin, corn starch, gum tragacanth, sodium alginate, or polyethylene glycol (PEG). Examples of sweeteners include sucrose, lactose, glucose, aspartame or saccharine. Disintegrating agents include corn starch, methylcellulose, polyvinylpyrrolidone, xanthan gum, bentonite, alginic acid or agar. Examples of diluents include lactose, sorbitol, mannitol, dextrose, kaolin, cellulose, calcium carbonate, calcium silicate or dicalcium phosphate. Flavoring agents include peppermint oil, oil of wintergreen, cherry, orange, or raspberry flavoring. Coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Examples of excipients may further include a polymer selected from the group consisting of polyvinylpyrrolidone (PVP), polyvinylpyrrolidone-vinylacetate copolymer (PVP-VA), hydroxypropylmethylcellulose (HPMC), hypromellose-acetate-succinate (HPMCAS), and mixtures thereof.

The present compositions may also be employed as fillers in soft and hard-filled capsules. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that release the active ingredient(s) only, or preferentially, in a certain part of the intestinal tract, optionally, in a delayed manner. Examples of embedding compositions that can be used include polymeric substances and waxes. The composition may also be in micro-encapsulated form with one or more excipients as noted above.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, these oral compositions can also include adjuvants such as wetting agents, emulsifying and suspending agents, surfactants, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a provided compound, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled.

Examples of other biodegradable polymers include poly(orthoesters) and poly(anhydrides). Depot injectable formulations are also prepared by entrapping the compound in liposomes or microemulsions that are compatible with body tissues.

Dosage forms for topical or transdermal administration of a compound of the present description include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of the present description. Additionally, the description contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutically acceptable compositions provided herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promotors to enhance bioavailability, fluorocarbons, and/or other conventional solubilizing or dispersing agents.

The amount of compound that may be combined with carrier materials to produce a composition in a single dosage form will vary depending upon the patient to be treated and the particular mode of administration.

As used herein, the term “effective amount” means that amount of a compound that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in treatment, healing, prevention, or amelioration of a disease, disorder, or symptom thereof, or a decrease in the rate of advancement of a disease or disorder. The term also includes within its scope amounts effective to enhance normal physiological function.

As used herein, the terms “treatment”, “treat”, and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “patient” or “subject” as used herein refers to an animal such as a mammal. A subject may therefore refer to, for example, mice, rats, dogs, cats, horses, cows, pigs, guinea pigs, primates including humans and the like. Preferably the subject is a human.

The present compounds are useful for the treatment of diseases and disorders where inhibition of the cannabinoid receptor CB₁ is indicated. Accordingly, here are contemplated a use of the present compounds for the treatment of a disease or disorder as defined herein, a use of the present compounds in the manufacture of a medicament for the treatment of a disease or disorder as defined herein, a compound as herein defined for use in the treatment of the present diseases or disorders, as well as a method for treating a disease or disorder as defined herein comprising the administration of one of the present compounds to a subject in need thereof. Such diseases and disorders may generally be generally related to diabetes and metabolic disorders (e.g. metabolic syndrome). Preferably, the compound selectively targets the CB₁ receptor in peripheral tissue (e.g. adipose tissue, liver, muscle, lung, kidney, macrophages, pancreatic beta cells and gastrointestinal tract), while not or mainly not interacting with CB₁ receptors in brain tissue, thereby avoiding or reducing CNS-related side effects.

The effect of the present compounds may include reduced food intake, reduced body weight, reversed insulin and leptin resistance, reverse hepatic steatosis (fatty liver) and improved dyslipidemia. Examples of diseases and disorders to be treated include obesity, diabetes (type I or II), non-alcoholic and alcoholic fatty liver disease (a risk factor for insulin resistance), a co-morbidity of obesity, a co-morbidity of diabetes, Prader-Willi Syndrome (PWS), Pro-opiomelanocortin (POMC) deficiency obesity, leptin receptor (LepR) deficiency obesity, POMC heterozygous deficiency obesity, POMC epigenetic disorders, Bardet-Biedl (BB) syndrome, Alstr6m syndrome, dyslipidemia predisposing to arteriosclerotic heart disease, diabetic nephropathy, fibrosis and fibrotic diseases of the skin, liver, lung or kidney such as Idiopathic Pulmonary Fibrosis (IPF), Progressing Fibrosis Interstitial Lung Diseases, Hermansky-Pudlak Syndrome pulmonary fibrosis (HPS-PF), cirrhosis, renal fibrosis, scleroderma, and gout. In addition, disorders of the skin include reducing scar formation (cicatrix, keloid) and alopecia, particularly that associated with male pattern baldness and metabolic syndrome. For instance, the co-morbidity of obesity is selected from metabolic syndrome, dementia, heart disease, hypertension, gallbladder disease, gastrointestinal disorders, menstrual irregularities, degenerative arthritis, venous statis ulcer, pulmonary hypoventilation syndrome, sleep apnea, snoring, obese asthma, coronary artery disease, arterial sclerotic disease, pseudotumor cerebri, osteoarthritis, high cholesterol, and increased incidence of malignancies of the liver, ovaries, cervix, uterus, breasts, prostate, or gallbladder. In preferred examples, the disease or disorder include diabetes (type I or II), obesity, and non-alcoholic fatty liver disease (e.g. non-alcoholic steatohepatitis). Examples of co-morbidities of diabetes (e.g. type 1) include diabetic nephropathy, chronic kidney disease, diabetic retinopathy, and peripheral and autonomic neuropathy.

Diseases, disorders and conditions to be treated, including those above, may be separated into various categories, while some of the conditions may also coexist in one given subject. Examples of categories include disorders related to appetite and their complications, disorders related to glucose regulation and their complications, fibrosis related disorders and their complications, disorders related to metabolism and their complications, disorders related to skin and hair growth and healing, disorders related to the GI tract, and disorders related to obesity and their complications.

Examples of disorders related to appetite and their complications include, without limitation, Prader-Willi Syndrome (PWS), hypothalamic obesity, pro-opiomelanocortin (POMC) deficiency (including POMC obesity, heterozygous POMC deficiency obesity, POMC epigenetic disorders), leptin receptor (LepR) deficiency, Bardet-Biedl (BB) syndrome, and Alstr6m syndrome.

Examples of disorders related to glucose regulation and their complications include, without limitation, diabetes Type I, diabetes Type II, insulin resistance, pre-diabetes, pancreatic diseases (by β-cell protection and/or increased insulin production), and associated nephropathies, neuropathies and retinopathies.

Examples of fibrosis related disorders and their complications include, without limitation, progressive fibrosis associated with interstitial lung disease, idiopathic pulmonary fibrosis (IPF), Hermansky-Pudlak syndrome pulmonary fibrosis (HPS-PF), cirrhosis and other liver fibrosis disorders (such as nonalcoholic steatohepatitis (NASH), primary sclerosing cholangitis, primary biliary cholangitis), fibrotic renal diseases, skin fibrotic disorders (such as scleroderma, and chronic kidney diseases.

Examples of disorders related to metabolism and their complications include, without limitation, metabolic syndrome and hyperlipidemia (e.g. hyper-triglyceridemia, hyper-triglyceridemia in the setting of low HDL-cholesterol, elevation of LDL and/or total cholesterol and/or VLDL and/or elevated Apolipoprotein B, atherosclerotic cardiovascular disease, etc.).

Examples of disorders related to obesity and their complications include, without limitation, sleep apnea, snoring, asthma, pulmonary hypoventilation syndrome, dementia, heart disease, hypertension, gallbladder disease, gastrointestinal disorders, menstrual irregularities, degenerative arthritis, venous statis ulcer, coronary artery disease, arterial sclerotic disease, pseudotumor cerebri, osteoarthritis, high cholesterol, and increased incidence of malignancies of the liver, ovaries, cervix, uterus, breasts, prostate, or gallbladder.

Examples of disorders of the skin and hair include alopecia (male pattern baldness and alopecia associated with metabolic syndrome), excessive scar formation (cicatrix and keloid), scleroderma, among others.

Examples of disorders related to the GI tract include constipation, irritable bowel syndrome, inflammatory bowel syndrome, including ulcerative colitis and Crohn's disease, etc.

Other disorders may also benefit from the present compounds, including muscle wasting disorders including muscular dystrophy (such as Duchenne Muscular Dystrophy (DMD)), amyotrophic lateral sclerosis (ALS), multiple sclerosis (MS), spinal muscular atrophy, and others.

The present solid compounds and compositions may also be used in a method for preventing or reversing the deposition of adipose tissue in a subject, which is expected to contribute to a reduction of incidence or severity of obesity, which in turn would reduce the incidence or severity of associated co-morbidities.

The present description provides a method of treating a disorder (as described herein) in a subject, comprising administering to the subject identified as in need thereof, a compound or composition of the present description. The identification of those patients who are in need of treatment for the disorders described above is well within the ability and knowledge of one skilled in the art. Certain of the methods for identification of patients which are at risk of developing the above disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination, medical/family history, and genetic determination.

A method of assessing the efficacy of a treatment in a subject includes determining the pre-treatment symptoms of a disorder by methods well known in the art and then administering a therapeutically effective amount of a compound of the present description, to the subject. After an appropriate period of time following the administration of the compound (e.g., 1 week, 2 weeks, one month, six months), the symptoms of the disorder are reevaluated. The modulation (e.g., decrease) of symptoms and/or of a biomarker of the disorder indicates efficacy of the treatment. The symptoms and/or biomarker of the disorder may be determined periodically throughout treatment. For example, the symptoms and/or biomarker of the disorder may be checked every few days, weeks or months to assess the further efficacy of the treatment. A decrease in symptoms and/or biomarker of the disorder indicates that the treatment is efficacious.

Pharmaceutical compositions provided herein are preferably adapted for oral administration. Such formulations may be administered with or without food. The compositions are formulated in unit dosage forms for ease of administration and uniformity of dosage. The expression “unit dosage form” as used herein refers to a physically discrete unit of agent appropriate for the patient to be treated. It will be understood, however, that the total daily usage of the solid dispersions and compositions of the present disclosure will be decided by the attending physician within the scope of sound medical judgment.

The amount of composition that may be included in a single dosage form will vary depending upon the patient to be treated (e.g. child vs adult, etc.) and the particular compound included in the composition. Provided compositions may be formulated such that a total daily dosage of, for instance, between 0.01 and 100 mg/kg body weight/day or between 0.01 and 20 mg/kg body weight/day of the compound can be administered to a patient receiving these compositions. Single dose compositions may contain such an amount, or the total daily dose may be divided in multiple dosage forms to be taken, for instance, one, two or three times a day. For instance, a single dose may include between 5 and 500 mg of the active ingredient, or between 20 and 200 mg. Treatment regimens may comprise administration to a patient a total amount of from about 10 mg to about 1000 mg of the compound(s) of the present description per day in a single dose or divided in multiple doses.

It will be understood that the total daily dose of the compound will be decided by the attending physician within the scope of sound medical judgment. For instance, a specific dosage or treatment regimen for any particular patient will depend upon a variety of factors, including age, body weight, general health, sex, diet, time of administration, rate of excretion, drug combination, the judgment of the treating physician, and the severity of the symptoms associated with the disease or disorder.

Depending upon the disease or disorder to be treated, additional therapeutic agents may also be present in the compositions of this disclosure or co-administered separately. Non-limiting examples of additional therapeutic agents which could be used in combination with the present solid dispersions and formulations include antidiabetic agents, cholesterol-lowering agents, anti-inflammatory agents, antimicrobial agents, matrix metalloproteinase inhibitors, lipoxygenase inhibitors, cytokine antagonists, immunosuppressants, anti-cancer agents, anti-viral agents, cytokines, growth factors, immunomodulators, prostaglandins, or anti-vascular hyperproliferation compound. The treatment may also be complemented with other treatments or interventions such as surgery, radiotherapy (e.g., gamma-radiation, neutron beam radiotherapy, electron beam radiotherapy, proton therapy, brachytherapy, and systemic radioactive isotopes), a biologic response modifier (e.g., an interferon, an interleukin, tumor necrosis factor (TNF)), and agents used to attenuate an adverse effect of the present compound or of a co-administered ingredient.

The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

The following non-limiting examples are illustrative embodiments and should not be construed as further limiting the scope of the present invention. These examples will be better understood with reference to the accompanying figures.

Unless otherwise indicated, all numbers expressing quantities of ingredients, reaction conditions, concentrations, properties, stabilities, and so forth used in the specification and claims are to be understood as being modified in all instances by the term “about.” At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

### Example 1—Synthesis of Intermediate I-1

Scheme 1 illustrates an example of synthesis for preparing Intermediate I-1 as shown below.

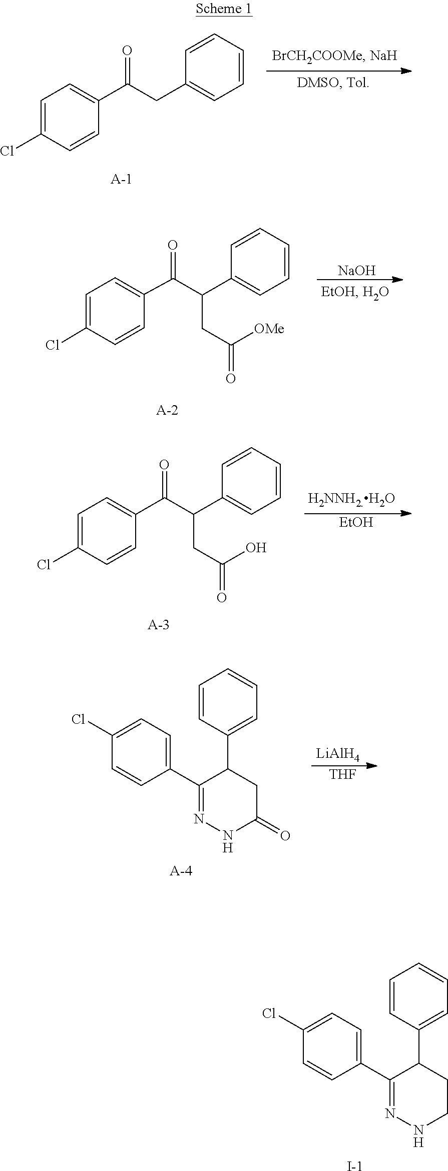

### Step-1: Synthesis of compound A-2

A mixture of sodium hydride (3.5 g, 86.7 mmol, 60% purity, 1 eq.) in dimethylsulfoxide (45 mL) was added a solution of 1-(4-chlorophenyl)-2-phenyl-ethanone A-1 (20 g, 86.7 mmol, 1 eq.) in dimethylsulfoxide (111 mL). Then a solution of methyl 2-bromoacetate (13.3 g, 86.7 mmol, 8.2 mL, 1 eq.) in toluene (68 mL) was added and the mixture was stirred at 25° C. for 16 h. The mixture was poured to hydrochloric acid (1M, 200 mL) and extracted with ethyl acetate 600 mL (3×200 mL). Then the organic phase was washed with brine (500 mL), dried over anhydrous sodium sulfate and concentrated to give the residue. The residue was triturated with petroleum ether (100 mL) to give the product methyl 4-(4-chlorophenyl)-4-oxo-3-phenyl-butanoate A-2 (17.0 g) as a light yellow solid.

¹H NMR (400 MHz, DMSO-d₆) δ=8.08-7.99 (m, 2H), 7.57-7.51 (m, 2H), 7.36-7.27 (m, 4H), 7.24-7.18 (m, 1H), 5.25 (m, 1H), 3.57 (s, 3H), 3.23 (m, 1H), 2.74 (m, 1H).

### Step-2: Synthesis of compound A-3

To a solution of methyl 4-(4-chlorophenyl)-4-oxo-3-phenyl-butanoate A-2 (17 g, 56.2 mmol, 1 eq.) in ethanol (35 mL) was added a solution of sodium hydroxide (22.5 g, 561.5 mmol, 10 eq.) in water (35 mL). Then the mixture was stirred at 25° C. for 2 h. After evaporation of ethanol, the residue was diluted with water (200 mL) and washed with ethyl acetate (3×50 mL). The aqueous layer was then acidified by 6N hydrochloric acid (50 mL) and extracted with ethyl acetate (3×200 mL). After drying over sodium sulfate, and evaporation of the solvent, the product 4-(4-chlorophenyl)-4-oxo-3-phenyl-butanoic acid A-3 (13 g) was obtained as a light yellow solid.

¹H NMR (400 MHz, DMSO-d₆) δ=12.31 (br s, 1H), 8.03 (d, J=8.8 Hz, 2H), 7.53 (d, J=8.8 Hz, 2H), 7.35-7.26 (m, 4H), 7.23-7.17 (m, 1H), 5.19 (m, 1H), 3.16 (m, 1H), 2.63 (m, 1H).

### Step-3: Synthesis of compound A-4

To a solution of 4-(4-chlorophenyl)-4-oxo-3-phenyl-butanoic acid A-3 (13 g, 45.0 mmol, 1 eq.) in ethanol (344 mL) was added hydrazine hydrate (90.1 mmol, 4.5 mL, 98% purity, 2 eq.). Then the mixture was stirred at 80° C. for 16 h. The mixture was cooled and filtered. The filter cake was dried to give 3-(4-chlorophenyl)-4-phenyl-4,5-dihydro-1 H-pyridazin-6-one A-4 (10.1 g, crude) as a white solid.

¹H NMR (400 MHz, DMSO-d₆) δ=11.22 (s, 1H), 7.74 (d, J=8.8 Hz, 2H), 7.44 (d, J=8.8 Hz, 2H), 7.35-7.29 (m, 2H), 7.27-7.22 (m, 1H), 7.20-7.16 (m, 2H), 4.68 (d, J=7.2 Hz, 1H), 3.09 (m, 1H), 2.47 (s, 1H).

### Step-4: Synthesis of Intermediate I-1

To a solution of 3-(4-chlorophenyl)-4-phenyl-4,5-dihydro-1 H-pyridazin-6-one A-4 (1 g, 3.5 mmol, 1 eq.) in tetrahydrofuran (10 mL) was added lithium aluminum hydride (400 mg, 10.5 mmol, 3 eq.) at 0° C. Then the mixture was stirred at 70° C. for 1 h. The mixture was filtered, and the filtrate was concentrated to give the residue. The residue was purified by silica column (petroleum ether: ethyl acetate, 5:1) to give the product 3-(4-chlorophenyl)-4-phenyl-1,4,5,6-tetrahydropyridazine (Intermediate I-1, 730 mg) as a yellow solid.

¹H NMR (400 MHz, DMSO-d₆) δ=7.67 (s, 1H), 7.50-7.43 (m, 2H), 7.32-7.12 (m, 7H), 4.15 (d, J=4.4 Hz, 1H), 3.10-3.03 (m, 1H), 2.92 (m, 1H), 2.19-2.06 (m, 1H), 1.87-1.77 (m, 1H).

### Example 2—Synthesis of Intermediates 1-2

Some of Intermediates 1-2 as defined below may be commercially available, while others may be prepared through various procedures. Schemes 2 to 4 illustrate three procedures that can be used for preparing Intermediates 1-2.

### (a) Procedure 1 (Scheme 2):

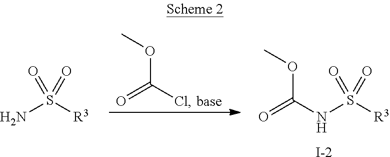

### i. Methyl N-(4-chlorophenyl)sulfonylcarbamate (R³=4-CI-Ph)

To a solution of 4-chlorobenzenesulfonamide (R³=4-CI-Ph, 5 g, 26.1 mmol, 1 eq.) and potassium carbonate (10.4 g, 74.9 mmol, 2.87 eq.) in acetone (35 mL) and water (2.3 mL) was added methyl chloroformate (36.53 mmol, 2.8 mL, 1.4 eq.) at 0° C. Then the mixture was stirred at 25° C. for 2 hours. Then the pH was adjusted to 3 with 1M hydrochloric acid (50 mL) and the mixture was extracted with ethyl acetate 90 mL (3×30 mL). Then the organic phase was washed with brine (50 mL), dried over anhydrous sodium sulfate and concentrated to give crude product methyl N-(4-chlorophenyl)sulfonylcarbamate 1-2 (R³=4-CI-Ph, 5.8 g) as a white solid.

¹H NMR (400 MHz, DMSO-d₆) δ=12.61-11.72 (m, 1H), 7.91 (d, J=8.8 Hz, 2H), 7.70 (d, J=8.8 Hz, 2H), 3.58 (s, 3H).

### ii. Methyl N-(3,5-difluorophenyl)sulfonylcarbamate (R³=3,5-F₂-Ph)

To a solution of 3,5-difluorobenzenesulfonamide (R³=3,5-F₂-Ph, 2 g, 10.35 mmol, 1 eq.) in DCM (20 mL) was added triethylamine (31.06 mmol, 4.3 mL, 3 eq) at 0° C. and stirred at 0° C. for 10 min. Then to the mixture was added methyl chloroformate (1.17 g, 12.42 mmol, 0.96 mL, 1.2 eq) at 0° C. The mixture was stirred at 0° C. for 30 min and then warmed up to 25° C. and stirred for 2 hours. The mixture was diluted with a sodium bicarbonate aqueous solution (30 mL), extracted with dichloromethane (30 mL×3) and washed with brine (5 mL). The organic layer was dried with anhydrous sodium sulfate, filtered and concentrated under vacuum to give the crude product methyl N-(3,5-difluorophenyl)sulfonylcarbamate 1-2 (R³=3,5-F₂-Ph, 4.4 g, crude) as a yellow solid.

¹H NMR (400 MHz, Chloroform-d) 6=7.52-7.44 (m, 2H), 6.92-6.84 (m, 1H), 3.58-3.53.

### iii. Methyl N-[5-(trifluoromethyl)-2-pyridyl]sulfonylcarbamate (R³=5-CF₃-2-Py)

To a solution of 5-(trifluoromethyl)pyridine-2-sulfonamide (1 g, 4.42 mmol, 1 eq.) and potassium carbonate (1.8 g, 12.7 mmol, 2.87 eq) in acetone (10 mL) and water (2 mL) was added methyl chloroformate (585 mg, 6.2 mmol, 0.5 mL, 1.4 eq) at 0° C. Then the mixture was stirred at 25° C. for 2 h. The mixture was filtered and the filtrate was concentrated under reduced pressure to give methyl N-[5-(trifluoromethyl)-2-pyridyl]sulfonylcarbamate 1-2 (R³=5-CF₃-2-Py, 1.3 g, crude) as a light yellow solid.

¹H NMR (400 MHz, DMSO-d₆) δ=8.96 (s, 1H), 8.32 (d, J=8.0 Hz, 1H), 8.05 (d, J=8.0 Hz, 1H), 3.30 (s, 3H).

### (b) Procedure 2 (Scheme 3)

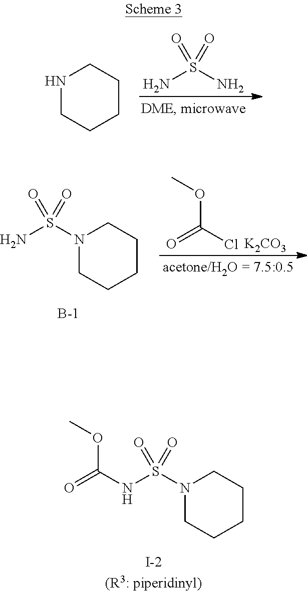

### Step 1-Synthesis of compound B-1

A mixture of piperidine (11.7 mmol, 1.2 mL, 1 eq.) and sulfamide (6.8 g, 70.5 mmol, 6 eq.) in dimethoxyethane (10 mL) was stirred at 120° C. for 3 h under microwave. The mixture was concentrated to give a residue. The residue was purified by silica column (petroleum ether:ethyl acetate=1:1, Rf=0.4) to give the product piperidine-1-sulfonamide B-1 (1.3 g) as a white solid.

¹H NMR (400 MHz, DMSO-d₆) δ=6.65 (s, 2H), 2.97-2.87 (m, 4H), 1.56 (m, 4H), 1.46-1.39 (m, 2H).

### Step 2-Synthesis of Intermediate 1-2 (with R³: N-piperidinyl)

To a solution of piperidine-1-sulfonamide B-1 (1.3 g, 7.9 mmol, 1 eq.) and potassium carbonate (3.1 g, 22.7 mmol, 2.87 eq.) in acetone (9.75 mL) and water (0.65 mL) was added methyl chloroformate (11.1 mmol, 0.86 mL, 1.4 eq.) at 0° C. Then the mixture was stirred at 25° C. for 2h. Then the pH was adjusted to 3 with 1M hydrochloric acid (30 mL) and the mixture was extracted with ethyl acetate 90 mL (3×30 mL). Then the organic phase was washed with brine (30 mL), dried over anhydrous sodium sulfate and concentrated under reduced pressure to give the crude product methyl N-(1-piperidylsulfonyl)carbamate (1.8 g, crude) as a yellow oil.

¹H NMR (400 MHz, DMSO-d₆) δ=11.23 (s, 1H), 3.64 (s, 3H), 3.23-3.13 (m, 4H), 1.57-1.44 (m, 6H).

### (c) Procedure 3 (Scheme 4)

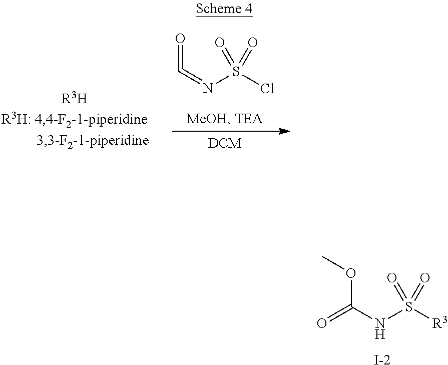

### i. Methyl N-[(4,4-difluoro-1-piperidyl)sulfonyl]carbamate (R³=4,4-F₂-piperidinyl)

To a solution of N-(oxomethylene)sulfamoyl chloride also known as chlorosulfonyl isocyanate (18.2 mmol, 1.6 mL, 1.1 eq.) in dichloromethane (16 mL) was added a solution of methanol (18.2 mmol, 0.7 mL, 1.1 eq.) in dichloromethane (3 mL) at −10° C. under nitrogen, the mixture was stirred at −10° C. for 1h. A solution of 4,4-difluoropiperidine (2 g, 16.5 mmol, 1 eq) and triethylamine (33.0 mmol, 4.6 mL, 2 eq.) in dichloromethane (3 mL) was added at −10° C., the mixture was stirred at 25° C. for 15 h. The mixture was concentrated to give the residue. The residue was purified by silica column (petroleum ether:ethyl acetate=1:1) to give the product methyl N-[(4,4-difluoro-1-piperidyl)sulfonyl]carbamate (1.4 g) as a white solid.

¹H NMR (400 MHz, DMSO-d₆) δ=11.54 (br s, 1H), 3.67 (s, 3H), 3.40-3.35 (m, 4H), 2.19-1.99 (m, 4H).

### ii. Methyl N-[(3,3-difluoro-1-piperidyl)sulfonyl]carbamate (R³=3,3-F₂-piperidinyl)

To a solution of N-(oxomethylene)sulfamoyl chloride (14.0 mmol, 1.2 mL, 1.1 eq.) in dichloromethane (16 mL) was added a solution of methanol (14.0 mmol, 0.56 mL, 1.1 eq.) in dichloromethane (3 mL) at −10° C. under nitrogen, the mixture was stirred at −10° C. for 1h. A solution of 3,3-difluoropiperidine (2.00 g, 12.69 mmol, 1 eq, HCl) and triethylamine (38.1 mmol, 5.3 mL, 3 eq.) in dichloromethane (3 mL) was added at −10° C., the the mixture was stirred at 25° C. for 15h.

The mixture was concentrated to give the residue. The residue was purified by silica column (petroleum ether:ethyl acetate=1:1) to give the product methyl N-[(3,3-difluoro-1-piperidyl)sulfonyl]carbamate (1.1 g) as a yellow oil.

¹H NMR (400 MHz, DMSO-d₆) δ=11.74 (s, 1H), 3.66 (s, 3H), 3.52 (t, J=11.6 Hz, 2H), 3.30-3.22 (m, 2H), 2.08-2.00 (m, 2H), 1.80-1.66 (m, 2H).

### Example 3—Synthesis of Compounds 1 to 8 and separation of isomers

Scheme 5 illustrates a general chemical synthesis of the present compound, such as Compounds 1 to 8, and separation of their isomers. It should be noted that isomers are numbered hereinbelow as Isomers 1 and 2, following the order in which they come out during chiral separation as presented below. This numbering should not be considered as an admission of an A or B compound structure as described herein and is not associated to the stereochemistry order indicated in Scheme 5, unless otherwise indicated.

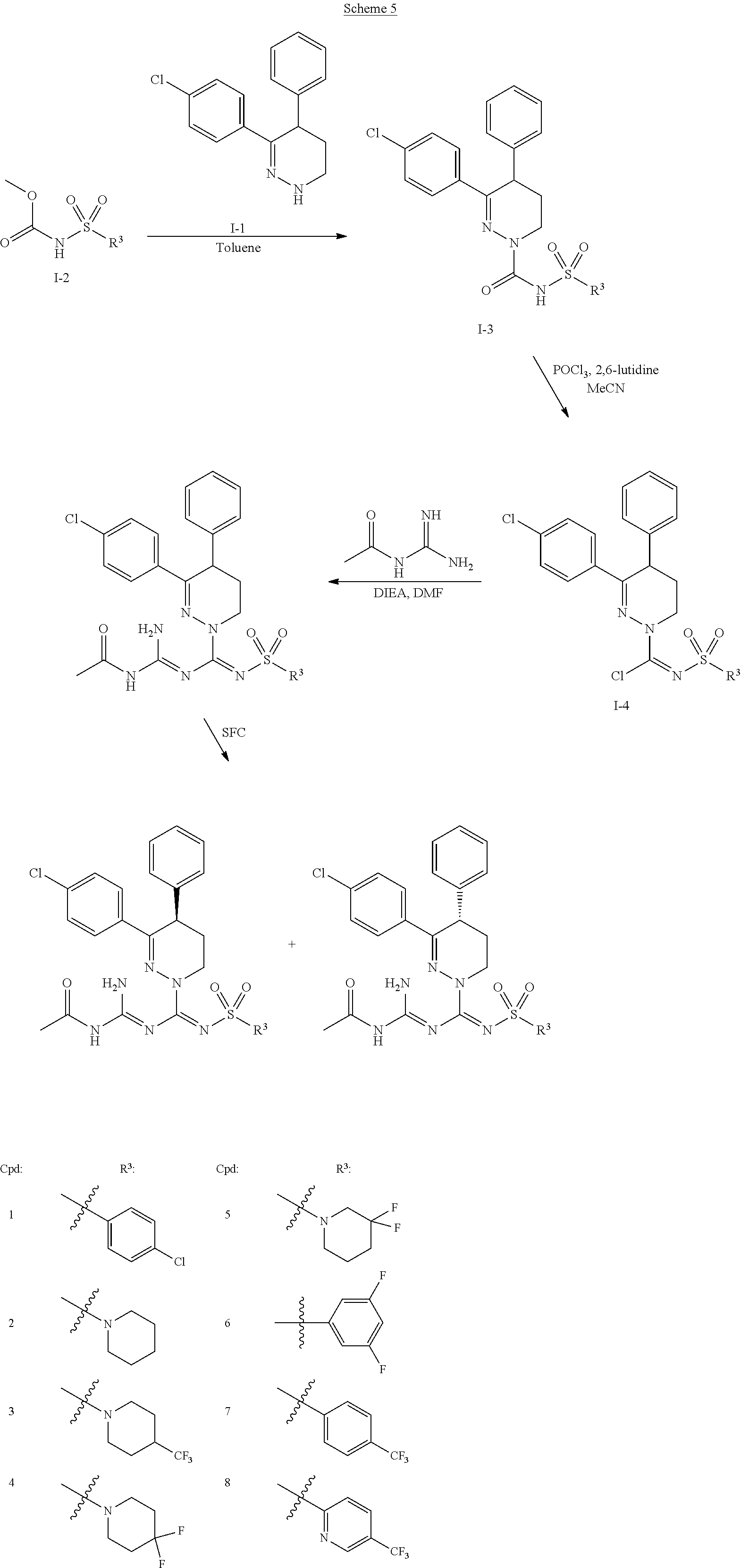

### (a) Compound 1

Compound 1 (and its isomers), where R³ is a 4-chlorophenyl group is prepared according to the following procedures.

### i. Intermediate 1-3 (R³=4-chlorophenyl)

A mixture of Intermediate 1-2 (Example 2(a)(i), 554.3 mg, 2.2 mmol, 1.2 eq.) and Intermediate 1-1 (Example 1, 500 mg, 1.9 mmol, 1 eq.) in toluene (20 mL) was stirred at 120° C. for 16 h. The mixture was concentrated under reduced pressure to give the residue. The residue was purified by silica column (petroleum ether:ethyl acetate=5:1) to give Intermediate 1-3, i.e. 6-(4-chlorophenyl)-N-(4-chlorophenyl)sulfonyl-5-phenyl-4,5-dihydro-3H-pyridazine-2-carboxamide (800 mg) as a yellow solid.

¹H NMR (400 MHz, DMSO-d₆) δ=11.23 (s, 1H), 8.05 (d, J=8.8 Hz, 2H), 7.89 (d, J=8.8 Hz, 2H), 7.74 (d, J=8.8 Hz, 2H), 7.42 (d, J=8.8 Hz, 2H), 7.35-7.27 (m, 2H), 7.26-7.18 (m, 1H), 7.13 (d, J=7.2 Hz, 2H), 4.46 (d, J=3.2 Hz, 1H), 3.97 (d, J=13.2 Hz, 1H), 2.90 (m, 1H), 2.17-2.05 (m, 1H), 1.94 (m, 1H).

### ii. Intermediate 1-4 (R³=4-chlorophenyl)

To a solution of Intermediate 1-3 from step (i) (400 mg, 819.0 μmol, 1 eq.) in acetonitrile (10 mL) was added phosphorus oxychloride (2.9 mmol, 0.27 mL, 3.5 eq.), and the mixture was stirred at 25° C. for 0.5h. Then 2,6-lutidine (3.3 mmol, 0.38 mL, 4 eq.) was added and the mixture was stirred at 80° C. for 15.5h. The mixture was poured into a sodium bicarbonate aqueous solution (20 mL) and extracted with ethyl acetate 60 mL (3×20 mL). Then the organic phase was washed with brine (20 mL), dried over anhydrous sodium sulfate and concentrated to give Intermediate 1-4 (2E)-6-(4-chlorophenyl)-N-(4-chlorophenyl)sulfonyl-5-phenyl-4,5-dihydro-3H-pyridazine-2-carboximidoyl chloride (400 mg, crude) as a brown solid.

### iii. Compound 1 (R³=4-chlorophenyl) racemate

To a solution of N-carbamimidoylacetamide (279 mg, 2.8 mmol, 4 eq.) and Intermediate 1-4 from step (ii) (350 mg, 690.6 μmol, 1 eq.) in N,N-dimethylformamide (3.5 mL) was added N,N-diisopropylethylamine (3.5 mmol, 0.60 mL, 5 eq.). Then the mixture was stirred at 25° C. for 2h. The mixture was purified by prep-HPLC (column: Waters Xbridge™ 150×25 mm, 5 um; mobile phase: [water(10 mM ammonium bicarbonate)-acetonitrile];B %: 49%-79%,10 min), and then the fraction was concentrated to remove acetonitrile and lyophilized to give the racemic Compound 1, i.e. N-[(E)-N′-[(Z)-C-[6-(4-chlorophenyl)-5-phenyl-4,5-dihydro-3H-pyridazin-2-yl]-N-(4-chlorophenyl)sulfonyl carbonimidoyl]carbamimidoyl]acetamide (180 mg) as a white solid.

### iv. Compound 1 (R³=4-chlorophenyl) isomers separation

The racemic product of step (iii) was separated by SFC (column: DAICEL CHIRALPAK™ AD(250 mm×30 mm,10 μm); mobile phase: 60% isopropanol (0.1% ammonia monohydrate) in CO₂, 3;50 min) to give Isomers 1 and 2 of Compound 1 having the following properties.

Isomer 1 (61 mg) as a white solid.

¹H NMR (400 MHz, DMSO-d₆) δ=10.67 (s, 1H), 8.34-7.70 (m, 4H), 7.54 (d, J=8.4 Hz, 4H), 7.33-7.26 (m, 4H), 7.25-7.14 (m, 3H), 4.38 (d, J=3.2 Hz, 1H), 4.31-4.22 (m, 1H), 3.23-3.12 (m, 1H), 2.28-2.18 (m, 1H), 2.13 (s, 3H), 1.97 (m, 1H).

LCMS: (ES*) m/z=571.1 (M+H), Rₜ=1.004 min.

SFC: ee value>99%, Rₜ=0.754 min.

Isomer 2 (51 mg) as a white solid.

¹H NMR (400 MHz, DMSO-d₆) δ=10.66 (s, 1H), 8.36-7.72 (m, 4H), 7.54 (d, J=8.8 Hz, 4H), 7.32-7.27 (m, 4H), 7.25-7.15 (m, 3H), 4.38 (d, J=3.2 Hz, 1H), 4.30-4.22 (m, 1H), 3.23-3.11 (m, 1H), 2.30-2.17 (m, 1H), 2.13 (s, 3H), 1.97 (m, 1H).

LCMS: (ES*) m/z=571.1(M+H), Rₜ=1.009 min.

SFC: ee value>99%, Rₜ=1.543 min.

### (b) Compound 2

Compound 2 (and its isomers), where R³ is a N-piperidinyl group is prepared according to the following procedures.

### i. Intermediate 1-3 (R³=N-piperidinyl)

A mixture of Intermediate 1-2 (Example 2(b), 493.4 mg, 2.22 mmol, 1.2 eq.) and Intermediate 1-1 (Example 1, 500 mg, 1.9 mmol, 1 eq.) in toluene (20 mL) was stirred at 120° C. for 16 h. The mixture was concentrated under reduced pressure to give the residue. The residue was triturated with ethanol (10 mL) to give Intermediate 1-3, i.e. 6-(4-chlorophenyl)-5-phenyl-N-(1-piperidylsulfonyl)-4,5-dihydro-3H-pyridazine-2-carboxamide (800 mg) as a yellow solid.

¹H NMR (400 MHz, DMSO-d₆) b=10.24 (s, 1H), 7.85 (d, J=8.8 Hz, 2H), 7.39 (d, J=8.8 Hz, 2H), 7.35-7.29 (m, 2H), 7.26-7.21 (m, 1H), 7.16 (d, J=7.2 Hz, 2H), 4.48 (d, J=3.2 Hz, 1H), 4.09 (d, J=13.2 Hz, 1H), 3.32-3.28 (m, 4H), 2.97 (m, 1H), 2.17 (m, 1H), 1.99 (m, 1H), 1.64-1.45 (m, 6H).

### ii. Intermediate 1-4 (R³=N-piperidinyl)

To a solution of Intermediate 1-3 from step (i) (400 mg, 867.7 μmol, 1 eq.) in acetonitrile (10 mL) was added phosphorus oxychloride (3.0 mmol, 0.28 mL, 3.5 eq.), and the mixture was stirred at 25° C. for 0.5h. Then 2,6-lutidine (3.5 mmol, 0.40 mL, 4 eq.) was added and the mixture was stirred at 80° C. for 15.5h. The mixture was poured into a sodium bicarbonate aqueous solution (20 mL) and extracted with ethyl acetate 60 mL (3×20 mL). Then the organic phase was washed with brine (20 mL), dried over anhydrous sodium sulfate and concentrated to give Intermediate 1-4, i.e. (2E)-6-(4-chlorophenyl)-5-phenyl-N-(1-piperidylsulfonyl)-4,5-dihydro-3H-pyridazine-2-carboximidoyl chloride (450 mg, crude) as a brown solid.

### iii. Compound 2 (R³=N-piperidinyl) racemate

To a solution of N-carbamimidoylacetamide (337.4 mg, 3.3 mmol, 4 eq.) and Intermediate 1-4 from step (ii) (400 mg, 834.3 μmol, 1 eq.) in N,N-dimethylformamide (4 mL) was added N,N-diisopropylethylamine (4.2 mmol, 0.73 mL, 5 eq.). Then the mixture was stirred at 25° C. for 2h.

The mixture was purified by prep-HPLC (column: Waters Xbridge™ 150×25 mm, 5 μm; mobile phase: [water(10 mM ammonium bicarbonate)-acetonitrile];B %: 42%-72%,9 min), and then the fraction was concentrated to remove acetonitrile and lyophilized to give the racemic Compound 2, i.e. N-[(E)-N′-[(Z)-C-[6-(4-chlorophenyl)-5-phenyl-4,5-dihydro-3H-pyridazin-2-yl]-N-(1-piperidylsulfonyl)carbonimidoyl]carbamimidoyl]acetamide (120 mg) as a white solid.

### iv. Compound 2 (R³=N-piperidinyl) isomers separation

The racemic product from step (iii) was separated by SFC (column: DAICEL CHIRALPAK™ AD(250 mm×30 mm,10 μm); mobile phase: 60% isopropanol (0.1% ammonia monohydrate) in CO₂, 4.3 min, 60 min) to give Isomers 1 and 2 of Compound 2 having the following properties.

Isomer 1 (54 mg) as a white solid.

¹H NMR (400 MHz, DMSO-d₆) δ=10.84 (s, 1H), 7.64 (d, J=8.8 Hz, 2H), 7.40-7.33 (m, 4H), 7.32-7.22 (m, 3H), 4.43 (d, J=3.2 Hz, 1H), 4.34-4.27 (m, 1H), 3.18 (br s, 1H), 3.10-3.02 (m, 4H), 2.41-2.27 (m, 1H), 2.21 (s, 3H), 2.03 (d, J=11.6 Hz, 1H), 1.60 (br s, 4H), 1.48 (d, J=4.4 Hz, 2H).

LCMS: (ES*) m/z=544.2 (M+H), Rₜ=0.992 min.

SFC: ee value>99%, Rₜ=0.616 min.

Isomer 2 (52 mg) as a white solid.

¹H NMR (400 MHz, DMSO-d₆) δ=10.84 (s, 1H), 7.64 (d, J=8.8 Hz, 2H), 7.40-7.34 (m, 4H), 7.31-7.23 (m, 3H), 4.43 (d, J=3.6 Hz, 1H), 4.31 (d, J=12.8 Hz, 1H), 3.25-3.14 (m, 1H), 3.11-3.04 (m, 4H), 2.37-2.27 (m, 1H), 2.21 (s, 3H), 2.07-1.99 (m, 1H), 1.60 (br s, 4H), 1.48 (d, J=4.4 Hz, 2H).

LCMS: (ES*) m/z=544.2(M+H), Rₜ=0.992 min.

SFC: ee value>99%, Rₜ=2.235 min.

### (c) Compound 3

Compound 3 (and its isomers), where R³ is a N-(4-trifluoromethyl-1-piperidinyl) group is prepared according to the following procedures.

### i. Intermediate 1-3 (R³=4-trifluoromethyl-1-piperidinyl)

A mixture of methyl N-[[4-(trifluoromethyl)-1-piperidyl]sulfonyl]carbamate as Intermediate 1-3 (500 mg, 1.7 mmol, 1 eq.) and Intermediate 1-1 (Example 1, 560 mg, 2.1 mmol, 1.2 eq.) in toluene (20 mL) was stirred at 120° C. for 16 h. The mixture was concentrated under reduced pressure to give a residue. The residue was purified by silica gel column (petroleum ether:ethyl acetate=5:1, R_{f}=0.5) to give Intermediate 1-3, i.e. 6-(4-chlorophenyl)-5-phenyl-N-[[4-(trifluoromethyl)-1-piperidyl]sulfonyl]-4,5-dihydro-3H-pyridazine-2-carboxamide (840 mg) as a yellow solid.

¹H NMR (400 MHz, DMSO-d₆) δ=10.41 (s, 1H), 7.86 (d, J=8.8 Hz, 2H), 7.39 (d, J=8.8 Hz, 2H), 7.34-7.29 (m, 2H), 7.26-7.20 (m, 1H), 7.15 (d, J=7.2 Hz, 2H), 4.48 (d, J=2.8 Hz, 1H), 4.12-4.03 (m, 1H), 3.86 (t, J=9.6 Hz, 2H), 3.10-2.87 (m, 4H), 2.23-2.11 (m, 1H), 2.06-1.96 (m, 1H), 1.90 (d, J=11.6 Hz, 2H), 1.57-1.41 (m, 2H).

### ii. Intermediate 1-4 (R³=4-trifluoromethyl-1-piperidinyl)

To a solution of Intermediate 1-3 from step (i) (400 mg, 756.2 μmol, 1 eq.) in acetonitrile (10 mL) was added phosphorus oxychloride (2.6 mmol, 0.25 mL, 3.5 eq.), and the mixture was stirred at 25° C. for 0.5h. Then 2,6-lutidine (3.0 mmol, 0.35 mL, 4 eq.) was added and the mixture was stirred at 80° C. for 15.5h. The mixture was poured into a sodium bicarbonate aqueous solution (20 mL) and extracted with ethyl acetate 60 mL (3×20 mL). Then the organic phase was washed with brine (20 mL), dried over anhydrous sodium sulfate and concentrated to give Intermediate 1-4, i.e. (2E)-6-(4-chlorophenyl)-5-phenyl-N-[[4-(trifluoromethyl)-1-piperidyl]sulfonyl]-4,5-dihydro-3H-pyridazine-2-carboximidoyl chloride (470 mg, crude) as a brown solid.

### iii. Compound 3 (R³=4-trifluoromethyl-1-piperidinyl) racemate

To a solution of N-carbamimidoylacetamide (347.4 mg, 3.4 mmol, 4 eq.) and Intermediate 1-4 from step (ii) (470 mg, 858.6 μmol, 1 eq.) in N,N-dimethylformamide (5 mL) was added N,N-diisopropylethylamine (4.3 mmol, 0.75 mL, 5 eq.). Then the mixture was stirred at 25° C. for 2h.

The mixture was purified by prep-HPLC (column: Waters Xbridge™ 150×25 mm, 5 μm; mobile phase: [water(10 mM ammonium bicarbonate)-acetonitrile];B %: 48%-78%,9 min), and then the fraction was concentrated to remove acetonitrile and lyophilized to give the racemic Compound 3, i.e. N-[(E)-N′-[(Z)-C-[6-(4-chlorophenyl)-5-phenyl-4,5-dihydro-3H-pyridazin-2-yl]-N-[[4-(trifluoromethyl)-1-piperidyl]sulfonyl]carbonimidoyl]carbamimidoyl]acetamide (180 mg) as a white solid.

### iv. Compound 3 (R³=4-trifluoromethyl-1-piperidinyl) isomers separation

The racemic product from step (iii) was separated by SFC (column: DAICEL CHIRALPAK™ AD(250 mm×30 mm,10 μm); mobile phase: 50% isopropanol (0.1% ammonia monohydrate) in CO₂, 3.6 min, 40 min) to give Isomers 1 and 2 of Compound 3 having the following properties.

Isomer 1 (80 mg) as a white solid.

¹H NMR (400 MHz, DMSO-d₆) δ=10.80 (s, 1H), 8.35-7.78 (m, 2H), 7.58 (d, J=8.8 Hz, 2H), 7.35-7.28 (m, 4H), 7.26-7.16 (m, 3H), 4.38 (d, J=3.2 Hz, 1H), 4.24 (d, J=13.2 Hz, 1H), 3.69-3.58 (m, 2H), 3.18-3.05 (m, 1H), 2.64 (d, J=12.4 Hz, 2H), 2.45-2.21 (m, 2H), 2.15 (s, 3H), 2.03-1.90 (m, 1H), 1.83 (d, J=12.8 Hz, 2H), 1.59-1.41 (m, 2H).

LCMS: (ES*) m/z=612.1 (M+H), Rₜ=1.049 min.

SFC: ee value>99%, Rₜ=0.537 min.

Isomer 2 (75 mg) as a white solid.

¹H NMR (400 MHz, DMSO-d₆) δ=10.86 (s, 1H), 8.42-7.79 (m, 2H), 7.64 (d, J=8.8 Hz, 2H), 7.40-7.34 (m, 4H), 7.32-7.23 (m, 3H), 4.44 (br d, J=3.2 Hz, 1H), 4.31 (d, J=13.2 Hz, 1H), 3.78-3.62 (m, 2H), 3.24-3.11 (m, 1H), 2.78-2.65 (m, 2H), 2.53-2.27 (m, 2H), 2.21 (s, 3H), 2.05-1.97 (m, 1H), 1.90 (d, J=12.4 Hz, 2H), 1.65-1.50 (m, 2H).

LCMS: (ES*) m/z=612.1(M+H), Rₜ=1.041 min.

SFC: ee value>99%, Rₜ=1.134 min.

### (d) Compound 4

Compound 4 (and its isomers), where R³ is a N-(4,4-difluoro-1-piperidinyl) group is prepared according to the following procedures.

### i. Intermediate 1-3 (R³=4,4-difluoro-1-piperidinyl)

A mixture of Intermediate 1-2 (Example 2(c)(i), 500 mg, 1.9 mmol, 1 eq.) and Intermediate 1-1 (Example 1, 524.2 mg, 1.9 mmol, 1.2 eq.) in toluene (5 mL) was stirred at 120° C. for 5 h. The mixture was concentrated under reduced pressure to give a residue. The residue was triturated with ethanol (10 mL) to give Intermediate 1-3, i.e. 6-(4-chlorophenyl)-N-[(4,4-difluoro-1-piperidyl)sulfonyl]-5-phenyl-4,5-dihydro-3H-pyridazine-2-carboxamide (800 mg) as a white solid.

¹H NMR (400 MHz, DMSO-d₆) δ=10.56 (s, 1H), 7.92-7.82 (m, 2H), 7.40 (d, J=8.8 Hz, 2H), 7.35-7.30 (m, 2H), 7.26-7.21 (m, 1H), 7.16 (d, J=7.2 Hz, 2H), 4.48 (d, J=3.2 Hz, 1H), 4.10 (d, J=12.8 Hz, 1H), 3.50 (m, 4H), 2.98 (m, 1H), 2.23-2.03 (m, 5H), 1.99 (m, 1H).

### ii. Intermediate 1-4 (R³=4,4-difluoro-1-piperidinyl)

To a solution of Intermediate 1-3 from step (i) (400 mg, 804.9 μmol, 1 eq.) in acetonitrile (5 mL) was added phosphorus oxychloride (2.8 mmol, 0.26 mL, 3.5 eq.), and the mixture was stirred at 25° C. for 0.5h. Then 2,6-lutidine (3.2 mmol, 0.37 mL, 4 eq.) was added and the mixture was stirred at 80° C. for 15.5h. The mixture was poured into water (20 mL) and extracted with ethyl acetate 30 mL (3×10 mL). Then the organic phase was washed with brine (30 mL), dried over anhydrous sodium sulfate and concentrated to give Intermediate 1-4, i.e. (2E)-6-(4-chlorophenyl)-N-[(4,4-difluoro-1-piperidyl)sulfonyl]-5-phenyl-4,5-dihydro-3H-pyridazine-2-carboximidoyl chloride (360 mg, crude) as a yellow solid.

### iii. Compound 4 (R³=4,4-difluoro-1-piperidinyl) racemate

To a solution of N-carbamimidoylacetamide (243.3 mg, 2.4 mmol, 4 eq.) and Intermediate 1-4 from step (ii) (310 mg, 601.5 μmol, 1 eq.) in N,N-dimethylformamide (3 mL) was added N,N-diisopropylethylamine (3.01 mmol, 0.52 mL, 5 eq.). Then the mixture was stirred at 25° C. for 16h.

The mixture was purified by prep-HPLC (column: Waters Xbridge™ 150×25 mm, 5 μm; mobile phase: [water(ammonium bicarbonate)-acetonitrile]; B %: 50%-80%, 10 min), and then the fraction was concentrated to remove acetonitrile and lyophilized to give the racemic Compound 4, i.e. N-[(E)-N′-[(Z)-C-[6-(4-chlorophenyl)-5-phenyl-4,5-dihydro-3H-pyridazin-2-yl]-N-[(4,4-difluoro-1-piperidyl)sulfonyl]carbonimidoyl]carbamimidoyl]acetamide (170 mg) as a yellow solid.

### iv. Compound 4 (R³=4,4-difluoro-1-piperidinyl) isomers separation

The racemic product from step (iii) was separated by SFC (column: DAICEL CHIRALPAK™ AD(250 mm×30 mm,10 μm); mobile phase: 45% isopropanol (0.1% ammonia monohydrate) in CO₂, 7.5 min, 90 min) to give Isomers 1 and 2 of Compound 4 having the following properties.

Isomer 1 (45 mg, 99.1% purity) as a white solid.

¹H NMR (400 MHz, DMSO-d₆) δ=10.83 (br s, 1H), 8.52-7.69 (m, 2H), 7.58 (d, J=8.8 Hz, 2H), 7.35-7.28 (m, 4H), 7.26-7.17 (m, 3H), 4.39 (d, J=3.6 Hz, 1H), 4.19 (d, J=13.2 Hz, 1H), 3.22 (t, J=5.2 Hz, 4H), 3.12 (t, J=11.6 Hz, 1H), 2.31-2.20 (m, 1H), 2.16 (s, 3H), 2.11-1.94 (m, 5H).

LCMS: (ES*) m/z=580.1 (M+H), Rₜ=1.000 min.

SFC: ee value>99%, Rₜ=1.793 min.

Isomer 2 (40 mg, 100% purity) as a white solid.

¹H NMR (400 MHz, DMSO-d₆) δ=10.83 (s, 1H), 8.52-7.72 (m, 2H), 7.58 (d, J=8.8 Hz, 2H), 7.35-7.28 (m, 4H), 7.26-7.16 (m, 3H), 4.39 (d, J=3.2 Hz, 1H), 4.19 (d, J=13.2 Hz, 1H), 3.22 (t, J=5.2 Hz, 4H), 3.17-3.07 (m, 1H), 2.31-2.20 (m, 1H), 2.16 (s, 3H), 2.13-1.95 (m, 5H).

LCMS: (ES*) m/z=580.1(M+H), Rₜ=1.003 min.

SFC: ee value>99%, Rₜ=2.257 min.

### (e) Compound 5

Compound 5 (and its isomers), where R³ is a N-(3,3-difluoro-1-piperidinyl) group is prepared according to the following procedures.

### i. Intermediate 1-3 (R³=3,3-difluoro-1-piperidinyl)

A mixture of Intermediate 1-2 (Example 2(c)(ii), 500 mg, 1.94 mmol, 1 eq.) and Intermediate 1-1 (Example 1, 524.2 mg, 1.9 mmol, 1 eq.) in toluene (5 mL) was stirred at 120° C. for 5 h. The mixture was concentrated under reduced pressure to give a residue. The residue was purified by silica column (petroleum ether:ethyl acetate=1:1) to give Intermediate 1-3, i.e. 6-(4-chlorophenyl)-N-[(3,3-difluoro-1-piperidyl)sulfonyl]-5-phenyl-4,5-dihydro-3H-pyridazine-2-carboxamide (800 mg) as a yellow solid.

¹H NMR (400 MHz, DMSO-d₆) δ=10.59 (s, 1H), 7.88 (d, J=8.8 Hz, 2H), 7.40 (d, J=8.8 Hz, 2H), 7.36-7.29 (m, 2H), 7.24 (d, J=7.2 Hz, 1H), 7.16 (d, J=7.2 Hz, 2H), 4.49 (d, J=2.8 Hz, 1H), 4.10 (d, J=13.2 Hz, 1H), 3.64 (t, J=11.6 Hz, 2H), 3.41-3.35 (m, 2H), 2.98 (m, 1H), 2.53 (d, J=1.6 Hz, 1H), 2.16 (m, 1H), 2.11-2.02 (m, 2H), 1.82-1.71 (m, 2H).

### ii. Intermediate 1-4 (R³=3,3-difluoro-1-piperidinyl)

To a solution of Intermediate 1-3 from step (i) (400 mg, 804.9 μmol, 1 eq.) in acetonitrile (5 mL) was added phosphorus oxychloride (2.8 mmol, 0.26 mL, 3.5 eq.), and the mixture was stirred at 25° C. for 0.5h. Then 2,6-lutidine (3.2 mmol, 0.37 mL, 4 eq.) was added and the mixture was stirred at 80° C. for 15.5h. The mixture was poured into water (20 mL) and extracted with ethyl acetate 30 mL (3×10 mL). Then the organic phase was washed with brine (30 mL), dried over anhydrous sodium sulfate and concentrated to give Intermediate 1-4, i.e. (2E)-6-(4-chlorophenyl)-N-[(3,3-difluoro-1-piperidyl)sulfonyl]-5-phenyl-4,5-dihydro-3H-pyridazine-2-carboximidoyl chloride (400 mg, crude) as a yellow solid.

### iii. Compound 5 (R³=3,3-difluoro-1-piperidinyl) racemate

To a solution of N-carbamimidoylacetamide (274.5 mg, 2.7 mmol, 4 eq.) and Intermediate 1-4 from step (ii) (350 mg, 679.1 μmol, 1 eq.) in N,N-dimethylformamide (3 mL) was added N,N-diisopropylethylamine (3.4 mmol, 0.59 mL, 5 eq.). Then the mixture was stirred at 25° C. for 16h. The mixture was purified by prep-HPLC (column: Waters Xbridge™ 150×25 mm, 5 μm; mobile phase: [water(ammonium bicarbonate)-acetonitrile]; B %: 48%-78%, 10 min), and then the fraction was concentrated to remove acetonitrile and lyophilized to give the racemic Compound 5, i.e. N-[(E)-N′-[(Z)-C-[6-(4-chlorophenyl)-5-phenyl-4,5-dihydro-3H-pyridazin-2-yl]-N-[(3,3-difluoro-1-piperidyl)sulfonyl]carbonimidoyl]carbamimidoyl]acetamide (140 mg) as a yellow solid.

### iv. Compound 5 (R³=3,3-difluoro-1-piperidinyl) isomers separation

The racemic product from step (iii) was separated by SFC (column: DAICEL CHIRALCEL™ OD(250 mm×30 mm,10 μm); mobile phase: 30% methanol (0.1% ammonia monohydrate) in CO₂, 3.0 min, 50 min) to give Isomers 1 and 2 of Compound 5 having the following properties.

Isomer 1 (35 mg, 99.5% purity) as a white solid.

¹H NMR (400 MHz, DMSO-d₆) δ=10.83 (br s, 1H), 8.58-7.70 (m, 2H), 7.58 (d, J=8.8 Hz, 2H), 7.35-7.28 (m, 4H), 7.26-7.17 (m, 3H), 4.39 (d, J=3.2 Hz, 1H), 4.26 (d, J=13.2 Hz, 1H), 3.28 (t, J=12.0 Hz, 2H), 3.19-3.12 (m, 1H), 3.11-3.05 (m, 2H), 2.31-2.21 (m, 1H), 2.16 (s, 3H), 2.04-1.90 (m, 3H), 1.71 (br s, 2H).

LCMS: (ES*) m/z=580.1 (M+H), Rₜ=1.008 min.

SFC: ee value>99%, Rₜ=1.467 min.

Isomer 2 (48 mg, 99.2% purity) as a white solid.

¹H NMR (400 MHz, DMSO-d₆) δ=10.84 (br s, 1H), 8.56-7.72 (m, 2H), 7.58 (d, J=8.8 Hz, 2H), 7.35-7.28 (m, 4H), 7.26-7.16 (m, 3H), 4.39 (d, J=3.2 Hz, 1H), 4.26 (d, J=13.2 Hz, 1H), 3.28 (t, J=11.6 Hz, 2H), 3.19-3.12 (m, 1H), 3.11-3.03 (m, 2H), 2.31-2.22 (m, 1H), 2.16 (s, 3H), 2.05-1.88 (m, 3H), 1.71 (br s, 2H).

LCMS: (ES*) m/z=580.1(M+H), Rₜ=1.003 min.

SFC: ee value>99%, Rₜ=1.648 min.

### (f) Compound 6

Compound 6 (and its isomers), where R³ is a 3,5-difluorophenyl group is prepared according to the following procedures.

### i. Intermediate 1-3 (R³=3,5-difluorophenyl)

A mixture of Intermediate 1-2 (Example 2(a)(ii), 510.29 mg, 2.03 mmol, 1.1 eq.) and Intermediate 1-1 (Example 1, 0.5 g, 1.85 mmol, 1 eq.) in toluene (5 mL) was stirred at 120° C. for 12 h. The mixture was diluted with water (10 mL), extracted with EtOAc (10 mL×2). The organic layer was dried with anhydrous sodium sulfate, filtered and concentrated under vacuum. The residue was purified by silica gel column chromatography (petroleum ether:ethyl acetate=2:1) to give Intermediate 1-3, i.e. 6-(4-chlorophenyl)-N-(3,5-difluorophenyl)sulfonyl-5-phenyl-4,5-dihydro-3H-pyridazine-2-carboxamide (0.5 g) as a brown solid.

¹H NMR (400 MHz, DMSO-d₆) δ=9.29 (s, 1H), 7.74 (d, J=4.2 Hz, 2H), 7.53 (d, J=8.6 Hz, 2H), 7.36-7.28 (m, 5H), 7.13-7.02 (m, 4H), 4.24-4.18 (m, 2H), 3.08 (m, 1H), 2.18-2.09 (m, 2H).

### ii. Intermediate 1-4 (R³=3,5-difluorophenyl)

To a solution of Intermediate 1-3 from step (i) (0.5 g, 1.02 mmol, 1 eq.) in acetonitrile (5 mL) was added phosphorus oxychloride (548 mg, 3.57 mmol, 0.33 mL, 3.5 eq.), and the mixture was stirred at 25° C. for 0.5h. Then 2,6-lutidine (437 mg, 4.08 mmol, 0.47 mL, 4 eq.) was added and the mixture was stirred at 40° C. for 2 h. The mixture was added dropwise into water (10 mL) and extracted with ethyl acetate 30 mL (3×10 mL). The organic phase was then dried over anhydrous sodium sulfate and concentrated under vacuum to give Intermediate 1-4 (2E)-6-(4-chlorophenyl)-N-(3,5-difluorophenyl)sulfonyl-5-phenyl-4,5-dihydro-3H-pyridazine-2-carboximidoyl chloride (0.5 g, crude) as a brown solid.

### iii. Compound 6 (R³=3,5-difluorophenyl) racemate

To a solution of N-carbamimidoylacetamide (89 mg, 885 μmol, 3 eq.) and Intermediate 1-4 from step (ii) (150 mg, 295.1 μmol, 1 eq.) in N,N-dimethylformamide (1.5 mL) was added N,N-diisopropylethylamine (1.48 mmol, 0.25 mL, 5 eq.). Then the mixture was stirred at 25° C. for 12h. The mixture was diluted with water (5 mL), extracted with ethyl acetate (5 mL×3). The organic layer was concentrated under vacuum. The residue was purified by prep-HPLC (0.1% M ammonium hydroxide), and then the fraction was lyophilized to give the racemic Compound 6, i.e. N-[(E)-N′-[(Z)-C-[6-(4-chlorophenyl)-5-phenyl-4,5-dihydro-3H-pyridazin-2-yl]-N-(3,5-difluorophenyl)sulfonyl carbonimidoyl]carbamimidoyl]acetamide (50 mg) as a white solid.

### iv. Compound 1 (R³=3,5-difluorophenyl) isomers separation

The racemic product of step (iii) was separated by SFC (column: DAICEL CHIRALCEL™ OD(250 mm×30 mm,10 μm); mobile phase: [Neu-MeOH];B %: 40%, 3.5; 35 min) to give Isomers 1 and 2 of Compound 6 having the following properties.

Isomer 1 (27 mg, 98.8% purity) as an off-white solid.

¹H NMR (400 MHz, Methanol-d₄) δ=7.57 (d, J=8.8 Hz, 2H), 7.49 (d, J=4.8 Hz, 2H), 7.34-7.28 (m, 2H), 7.24 (d, J=7.2 Hz, 1H), 7.21-7.12 (m, 5H), 4.59 (s, 2H), 4.44-4.29 (m, 2H), 2.37-2.25 (m, 1H), 2.14 (s, 3H), 2.09-2.05 (m, 1H). ¹⁹F NMR (377 MHz, Methanol-d₄) δ=−110.05 (br s, 1 F) LCMS: (ES*) m/z=573.2 (M+H), Rₜ=0.895 min.

SFC: ee value 97.88%, Rₜ=1.445 min.

Isomer 2 (31 mg) as an off-white solid.

¹H NMR (400 MHz, Methanol-d₄) δ=7.57 (d, J=8.8 Hz, 2H), 7.50-7.49 (m, 2H), 7.33-7.28 (m, 2H), 7.24 (d, J=7.2 Hz, 1H), 7.21-7.14 (m, 5H), 4.59 (s, 2H), 4.44-4.30 (m, 2H), 2.42-2.25 (m, 1H), 2.14 (s, 3H), 2.09-2.05 (m, 1H). ¹⁹F NMR (377 MHz, Methanol-d₄) δ=−110.05 (br s, 1 F) LCMS: (ES*) m/z=573.2(M+H), Rₜ=0.887 min.

SFC: ee value>99%, Rₜ=1.784 min.

### (g) Compound 7

Compound 7 (and its isomers), where R³ is a 4-trifluoromethylphenyl group is prepared according to the following procedures.

### i. Intermediate 1-3 (R³=4-trifluoromethylphenyl)

A mixture of Intermediate 1-2, methyl N-[4-(trifluoromethyl)phenyl]sulfonylcarbamate (500 mg, 1.8 mmol, 1 eq.) prepared by a known process, and Intermediate 1-1 (Example 1, 478 mg, 1.8 mmol, 1 eq.) in toluene (10 mL) was stirred at 120° C. for 12 h. After cooling, the mixture was concentrated under reduced pressure to give a residue. The residue was triturated with ethanol (10 mL), then filtered and the filter cake was dried to give Intermediate 1-3, i.e. 6-(4-chlorophenyl)-5-phenyl-N-[(4-trifluoromethyl)phenyl]sulfonyl-4,5-dihydro-3H-pyridazine-2-carboxamide (650 mg) as a light yellow solid.

¹H NMR (400 MHz, DMSO-d₆) δ=11.43 (br s, 1H), 8.26 (d, J=8.4 Hz, 2H), 8.06 (d, J=8.4 Hz, 2H), 7.89 (d, J=8.4 Hz, 2H), 7.43 (d, J=8.4 Hz, 2H), 7.35-7.27 (m, 2H), 7.26-7.18 (m, 1H), 7.13 (d, J=7.6 Hz, 2H), 4.47 (br d, J=3.2 Hz, 1H), 3.97 (d, J=12.8 Hz, 1H), 2.90 (dt, J=3.6, 13.2 Hz, 1H), 2.12 (m, 1H), 2.00-1.89 (m, 1H).

### ii. Intermediate 1-4 (R³=4-trifluoromethylphenyl)

To a solution of Intermediate 1-3 from step (i) (400 mg, 766.4 μmol, 1 eq.) in acetonitrile (4 mL) was added phosphorus oxychloride (2.7 mmol, 0.25 mL, 3.5 eq.), and the mixture was stirred at 25° C. for 0.5h. Then 2,6-lutidine (3.07 mmol, 0.36 mL, 4 eq.) was added and the mixture was stirred at 60° C. for 2.5h. The mixture was poured into water (10 mL) and extracted with ethyl acetate 15 mL (3×5 mL). Then the organic phase was washed with brine (10 mL), dried over anhydrous sodium sulfate and concentrated to give Intermediate 1-4, i.e. (2E)-6-(4-chlorophenyl)-5-phenyl-N-[4-(trifluoromethyl)phenyl]sulfonyl-4,5-dihydro-3H-pyridazine-2-carboximidoyl chloride (500 mg, crude) a brown solid.

### iii. Compound 7 (R³=4-trifluoromethylphenyl) racemate

To a solution of N-carbamimidoylacetamide (337 mg, 3.33 mmol, 4 eq.) and Intermediate 1-4 from step (ii) (450 mg, 832 μmol, 1 eq.) in N,N-dimethylformamide (4.5 mL) was added N,N-diisopropylethylamine (4.16 mmol, 0.72 mL, 5 eq.). Then the mixture was stirred at 25° C. for 1h.

The mixture was purified by prep-HPLC (column: Waters Xbridge™ 150×50 mm, 10 μm; mobile phase: [water(ammonium bicarbonate)-acetonitrile]; B %: 43%-73%, 11 min). The fraction was concentrated to remove acetonitrile and lyophilized to give the racemic Compound 7, i.e. N-[(E)-N′-[(Z)-C-[6-(4-chlorophenyl)-5-phenyl-4,5-dihydro-3H-pyridazin-2-yl]-N-[4-(trifluoromethyl)phenyl]sulfonyl-carbonimidoyl]carbamimidoyl]acetamide (170 mg) as a yellow solid.

### iv. Compound 7 (R³=4-trifluoromethylphenyl) isomers separation

The racemic product from step (iii) was separated by SFC (column: DAICEL CHIRALCEL™ OD(250 mm×30 mm,10 μm); mobile phase: 40% methanol (0.1% ammonia monohydrate) in CO₂, 3.5 min) to give Isomers 1 and 2 of Compound 7 having the following properties.

Isomer 1 (38 mg, 99% purity) as a white solid.

¹H NMR (400 MHz, Methanol-d₄) δ=8.11 (d, J=8.4 Hz, 2H), 7.83 (d, J=8.4 Hz, 2H), 7.58 (d, J=8.8 Hz, 2H), 7.34-7.29 (m, 2H), 7.25 (d, J=7.2 Hz, 1H), 7.18 (m, 4H), 4.41 (m, 1H), 4.33 (d, J=2.8 Hz, 1H), 3.31-3.27 (m, 1H), 2.37-2.26 (m, 1H), 2.14 (s, 3H), 2.09 (m, 1H).

LCMS: (ES*) m/z=605.2 (M+H), Rₜ=0.995 min.

SFC: ee value>99%, Rₜ=1.551 min.

Isomer 2 (38 mg, 99.6% purity) as a white solid.

¹H NMR (400 MHz, DMSO-d₆) δ=8.11 (d, J=8.4 Hz, 2H), 7.83 (d, J=8.4 Hz, 2H), 7.58 (d, J=8.8 Hz, 2H), 7.34-7.28 (m, 2H), 7.25 (d, J=7.2 Hz, 1H), 7.18 (m, 4H), 4.46-4.36 (m, 1H), 4.33 (d, J=3.2 Hz, 1H), 3.32-3.26 (m, 1H), 2.37-2.26 (m, 1H), 2.14 (s, 3H), 2.08 (m, 1H).

LCMS: (ES*) m/z=605.2 (M+H), Rₜ=0.997 min.

SFC: ee value>99%, Rₜ=1.923 min.

### (h) Compound 8

Compound 8 (and its isomers), where R³ is a 5-(trifluoromethyl)-2-pyridyl group is prepared according to the following procedures.

### i. Intermediate 1-3 (R³=5-(trifluoromethyl)-2-pyridyl)

A mixture of Intermediate 1-2 (Example 2(a)(iii), 1 g, 3.5 mmol, 1 eq.) was dissolved in N,N-dimethylformamide (10 mL) and Intermediate 1-1 (Example 1, 953 mg, 3.5 mmol, 1 eq.) was added. The mixture was stirred at 120° C. for 2 h. The mixture was poured into water (50 mL) and extracted with ethyl acetate 45 mL (3×15 mL). Then, the organic phase was washed with brine (50 mL), dried over anhydrous sodium sulfate and concentrated under reduced pressure to give a residue. The residue was purified by silica gel column (petroleum ether:ethyl acetate, 1:1) and prep-HPLC (formic acid, 0.1%, 30%-50% acetonitrile) to give Intermediate 1-3, i.e. 6-(4-chlorophenyl)-5-phenyl-N-[[5-(trifluoromethyl)-2-pyridyl]sulfonyl]-4,5-dihydro-3H-pyridazine-2-carboxamide (520 mg) as a yellow solid.

¹H NMR (400 MHz, DMSO-d₆) δ=12.00 (br s, 1H), 9.23 (s, 1H), 8.60 (d, J=7.6 Hz, 1H), 8.35 (d, J=8.4 Hz, 1H), 7.97 (d, J=8.4 Hz, 2H), 7.40 (d, J=8.4 Hz, 2H), 7.36-7.29 (m, 2H), 7.26-7.20 (m, 1H), 7.14 (d, J=7.2 Hz, 2H), 4.47 (br s, 1H), 3.93 (d, J=13.2 Hz, 1H), 2.89 (m, 1H), 2.20-2.07 (m, 1H), 1.97-1.87 (m, 1H).

### ii. Intermediate 1-4 (R³=5-(trifluoromethyl)-2-pyridyl)

To a solution of Intermediate 1-3 from step (i) (420 mg, 803.2 μmol, 1 eq.) in acetonitrile (4.2 mL) was added phosphorus oxychloride (2.8 mmol, 0.3 mL, 3.5 eq.), and the mixture was stirred at 25° C. for 0.5h. Then 2,6-lutidine (3.2 mmol, 0.37 mL, 4 eq.) was added and the mixture was stirred at 60° C. for 2.5h. After cooling, the mixture was poured into water (10 mL) and extracted with ethyl acetate 15 mL (3×5 mL). Then the organic phase was washed with brine (10 mL), dried over anhydrous sodium sulfate and concentrated to give Intermediate 1-4 (2E)-6-(4-chlorophenyl)-5-phenyl-N-[[5-(trifluoromethyl)-2-pyridyl]sulfonyl]-4,5-dihydro-3H-pyridazine-2-carboximidoyl chloride (540 mg, crude) as a yellow solid.

### iii. Compound 8 (R³=5-(trifluoromethyl)-2-pyridyl) racemate

To a solution of N-carbamimidoylacetamide (403 mg, 4 mmol, 4 eq.) and Intermediate 1-4 from step (ii) (540 mg, 997.5 μmol, 1 eq.) in N,N-dimethylformamide (5.4 mL) was added N,N-diisopropylethylamine (5 mmol, 0.87 mL, 5 eq.). Then, the mixture was stirred at 25° C. for 2 h. The mixture was poured into water (50 mL). The aqueous mixture was extracted with ethyl acetate 90 mL (3×30 mL). The organic layers were combined and washed with brine (50 mL), dried over anhydrous sodium sulfate and concentrated under reduce pressure to give a residue.

The residue purified by silica column (petroleum ether:ethyl acetate=1:1) and prep-HPLC (column: Phenomenex™ Luna C18 150×25 mm, 10 μm; mobile phase: [water(formic acid)-acetonitrile];B %: 45%-75%,10 min), and then the fraction was concentrated to remove acetonitrile and lyophilized to give the racemic Compound 8, i.e. N-[(NE)-N-[[6-(4-chlorophenyl)-5-phenyl-4,5-dihydro-3H-pyridazin-2-yl]-[[5-(trifluoromethyl)-2-pyridyl]sulfonylamino]methylene]carbamimidoyl]acetamide (95 mg) as a white solid.

### iv. Compound 8 (R³=5-(trifluoromethyl)-2-pyridyl) isomers separation

The racemic product of step (iii) was separated by SFC (column: DAICEL CHIRALPAK™ OD(250 mm×30 mm, 10 μm); mobile phase: 40% methanol (0.1% ammonia monohydrate) in CO₂, 4.6 min) to give Isomers 1 and 2 of Compound 1 having the following properties.

Isomer 1 (20 mg, 100% purity) as a white solid.

¹H NMR (400 MHz, DMSO-d₆) δ=10.48 (br s, 1H), 9.04 (s, 1H), 8.41 (dd, J=1.6, 8.4 Hz, 1H), 8.24-7.82 (m, 2H), 7.54 (d, J=4.8 Hz, 2H), 7.38-7.14 (m, 8H), 4.44-4.30 (m, 2H), 3.28-3.20 (m, 1H), 2.29-2.21 (m, 1H), 2.05 (s, 3H), 2.02-1.96 (m, 1H).

LCMS: (ES*) m/z=606.2 (M+H), Rₜ=0.950 min.

SFC: ee value>99%, Rₜ=1.698 min.

Isomer 2 (35 mg, 100% purity) as a white solid.

¹H NMR (400 MHz, DMSO-d₆) δ=10.48 (br s, 1H), 9.04 (s, 1H), 8.41 (dd, J=2.0, 8.4 Hz, 1H), 8.13 (d, J=8.4 Hz, 2H), 7.53 (m, 2H), 7.37-7.15 (m, 8H), 4.44-4.30 (m, 2H), 3.28-3.20 (m, 1H), 2.31-2.17 (m, 1H), 2.05 (s, 3H), 2.00 (m, 1H).

LCMS: (ES*) m/z=606.2(M+H), Rₜ=0.960 min.

SFC: ee value>99%, Rₜ=2.075 min.

### Example 4—Biological Properties

a) Design of the assay

### Day 1:

Using polyethylenimine (PEI) transfection agent, HEK293 cells were transfected in suspension with the human CB1 or CB1 b receptor and one of the following bioSensAll® assays: GAPL-Gi2, or β-arrestin plasma membrane (PM) translocation biosensor (+GRK2). Cells were directly seeded in 96-well plates immediately following transfection.

### Day 4 (˜65 Hours Post-Transfection):

Cells were incubated with coelenterazine (luciferase substrate) and different test compounds prior to the measurement of BRET signals.

b) BioSensAl© platform description.

Used in this project are i) Ga plasma membrane (GAPL) biosensors and the ii) β-arrestin plasma membrane translocation biosensor.
i) GAPL sensors are used to monitor the activation of heterotrimeric G proteins at the plasma membrane upon receptor stimulation. Specifically, these multimolecular BRET sensors detect the plasma membrane recruitment of proteins that interact with active Ga subunits in a G protein family-selective manner. G protein activation following receptor stimulation generally leads to an increase in the BRET signal.
ii) The multimolecular β-arrestin membrane recruitment biosensors allow for real-time spatio-temporal monitoring of β-arrestin 1 and β-arrestin 2 following GPCR activation. Specifically, these β-arrestin sensors were designed to detect the recruitment of proteins to the plasma membrane β-arrestin PM) with localization to compartment resulting in an increased BRET signal.
   c) Cell Culture Conditions HEK293 cells were maintained in Dulbecco's Modified Eagle Medium (DMEM) (Wisent; cat #319-030-CL: without sodium pyruvate, with 4.5 g/L glucose, without L-glutamine) supplemented with 1% penicillin-streptomycin (Wisent; cat #450-201-EL) and 2 or 10% fetal bovine serum (Wisent cat #090150).

### d) Transfection and Cell Plating

HEK293 cells were co-transfected with hCB1 or hCB1b and one of the above-listed bioSensAll® assays. For each transfection condition:
the total amount of transfected DNA was kept constant at 1 μg per mL of cell culture to be transfected; whenever necessary, salmon sperm DNA (Invitrogen, cat #15632011) was used as ‘carrier’ DNA to supplement plasmid DNA (i.e., biosensor and receptor);
the PEI (polyethylenimine 25 kDa linear, PolyScience, cat #23966) to DNA ratio (ug:ug) was fixed at 3:1.
   Transfections were Carried Out as Follows:
DNA and PEI were first diluted separately in 150 mM NaCl. The volume of the diluent in each tube corresponds to 5% of the cell culture volume to be transfected.
Once DNA and PEI were diluted, the PEI-containing solution was added to the DNA solution and the DNA/PEI mixture immediately vortexed for 5 seconds.
The DNA/PEI mixture was incubated for at least 20 minutes at room temperature to allow for the formation of DNA/PEI complexes.
During the incubation, HEK293 cells were detached, counted and re-suspended in culture medium (composition specified above).
At the end of the incubation period, the DNA/PEI mixture was added to the cells.
Cells were finally distributed in 96-well plates (White Opaque 96-well/Microplates, Greiner, cat #655083 or CulturPlate-96, White Opaque 96-well Microplate, PerkinElmer, cat #6005688) at a density of 30 000 cells per well.

### e) BRET Assay

### At −48 Hours Post-Transfection:

Using 450-Select TS Biotek plate washer, culture medium was aspirated and replaced with 100 μl of Hank's Balanced Salt Solution buffer (HBSS) (Wisent, cat #319-067CL: without red phenol; with sodium bicarbonate, with calcium and magnesium, with HEPES) per well. This process was performed a total of two times (i.e., 2 washes).
After the washing step, the HP D300 digital dispenser (Tecan) was used to inject increasing doses (12) of test compounds (ranging from 0-600 nM) to wells. Three to five minutes later, an EC75 of CP-55,940 (0.1 nM for GAPL-Gi2 assay; 100 nM for β-arrestin PM translocation biosensor (+GRK2)) was added to columns 2-12 of each 96-well plate.
The eight wells in column 1 of each plate were consistently reserved for controls (i.e., 3 non-treated wells, 2 wells injected with an EC75 of CP-55,940 and 3 wells injected with an EC100 of CP-55,940).
Plates were then incubated overnight (16-18 hours) at 37degC and 5% CO₂ in a humidified atmosphere.
The following day, BRET experiments were performed according to the following protocol:
10 μl of 20 μM e-Coelenterazine Prolume Purple (Methoxy e-CTZ) (Nanolight, cat #369) was added to each well for a final concentration of 2 μM.
Plates were incubated for 5 mins at room temperature on an orbital shaker.
BRET readings were then collected with a 0.4 sec integration time on a Synergy NEO plate reader (BioTek Instruments, Inc., USA; filters: 400 nm/70 nm, 515 nm/20 nm).

### f) Calculations

BRET signals were determined by calculating the ratio of light emitted by GFP-acceptor (515 nm) over light emitted by luciferase-donor (400 nm). All BRET ratios were standardized using the equation below with pre-established BRET values for positive and negative controls. The standardized BRET ratio is referred to as universal BRET (uBRET).

uBRET=((BRET ratio−A)/(B−A))*10 000

### Where:

A=BRET ratio obtained from transfection of negative control
B=BRET ratio obtained from transfection of positive control

Resulting dose-response curves were fitted using the four-parameter logistic non-linear regression model in GraphPad Prism 9.

Inv itro assay results for Isomers 1 and 2 of Compounds 1 to 8 as prepared in Example 3 are presented in Table 1.

TABLE 1
In vitro assay data

| | | CB₁ wt Gi2 | CB₁ wt βarr2 |
|---|---|---|---|
| Compound | Isomer | EC₅₀ (nM) | EC₅₀ (nM) |
| 1 | 1 | 430 | >30,000 |
| 1 | 2 | 1.2 | 3.3 |
| 2 | 1 | 300 | 23,600 |
| 2 | 2 | 0.65 | 1.5 |
| 3 | 1 | 190 | 14,100 |
| 3 | 2 | 0.64 | 1.3 |
| 4 | 1 | >30,000 | >30,000 |
| 4 | 2 | 0.67 | 1.4 |
| 5 | 1 | 150 | 400 |
| 5 | 2 | 0.43 | 0.72 |
| 6 | 1 | 39 | 380 |
| 6 | 2 | 0.68 | 2.6 |
| 7 | 1 | 200 | 580 |
| 7 | 2 | 0.42 | 2.1 |
| 8 | 1 | >30,000 | >30,000 |
| 8 | 2 | 1.9 | 8.5 |

For comparative purposes, the racemic compound identified as compound 3 in J. H. M. Lange et aL., Biorg. Med. Chem. Lett., 19 (2009), 5675-5678, was also prepared. The two isomers were separated by SFC as exemplified hereinabove and the two isomers were tested in vitro under the present conditions. The most active isomer was shown to have an Gi2 EC₅₀ of about 5.1 nM and a βarr2 EC₅₀ of 28 nM while the other isomer was inactive in both assays.

Numerous modifications could be made to any of the embodiments described above without departing from the scope of the present invention. Any references, patents or scientific literature documents referred to in the present document are incorporated herein by reference in their entirety for all purposes.

1. A compound of Formula I: 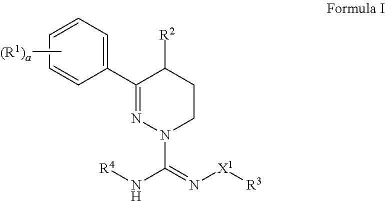
wherein,
R¹ is independently in each occurrence selected from optionally substituted alkyl, optionally substituted cycloalkyl, optionally substituted heterocycloalkyl, halogen, cyano, nitro, hydroxy, optionally substituted alkoxy, amino, optionally substituted alkylsulfonyl, optionally substituted aryl, optionally substituted heteroaryl, optionally substituted carboxyl, acyl, optionally substituted alkenyl, optionally substituted alkynyl, optionally substituted phosphonyl, optionally substituted phosphinyl, optionally substituted boronate, optionally substituted silyl, and imino;
R² and R³ are each independently selected from optionally substituted alkyl, aryl, heteroaryl, cycloalkyl, and heterocycloalkyl groups;
R⁴ is selected from —C(O)R⁵, —S(O)₂R⁵, —C(═NR⁶)R⁵, and —C(═NR⁶)NHC(O)R⁵;
R⁵ is selected from optionally substituted alkyl, optionally substituted alkoxy, optionally substituted alkylamino or dialkylamino, optionally substituted cycloalkyl, optionally substituted heterocycloalkyl, optionally substituted aryl, and optionally substituted heteroaryl;
R⁶ is selected from hydrogen, optionally substituted alkyl, optionally substituted alkoxy, optionally substituted alkylamino or dialkylamino, optionally substituted cycloalkyl, optionally substituted heterocycloalkyl, optionally substituted aryl, and optionally substituted heteroaryl, or
R⁶ is taken together with R⁵ and their adjacent atoms to form an optionally substituted heterocycloalkyl or heteroaryl;
X¹ is selected from SO₂ and C═O; and
a is 0, 1, 2, 3, 4 or 5;
or a tautomer thereof, or a pharmaceutically acceptable salt thereof.

2. The compound of claim 1, or a tautomer thereof or a pharmaceutically acceptable salt thereof, wherein said compound is of Formula I(a):

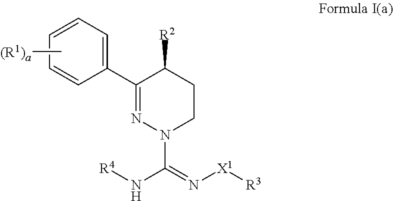

3. The compound of claim 1, or a tautomer thereof or a pharmaceutically acceptable salt thereof wherein said compound is of Formula I(b):

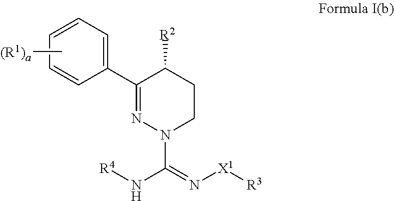

4-47. (canceled)

48. The compound of claim 1, or a tautomer thereof, or a pharmaceutically acceptable salt thereof, wherein R⁴ is —C(O)R⁵, —C(═NR⁶)R⁵, or —C(═NR⁶)NHC(O)R⁵; R⁵ is optionally substituted —C₁₋₆alkyl, optionally substituted —C₁₋₆alkoxy, optionally substituted —C₁₋₆alkylamino, or di —C₁₋₆alkylamino; and R⁶ is —H.

49. The compound of claim 48, or a tautomer thereof, or a pharmaceutically acceptable salt thereof, wherein R⁴ is —C(═NR⁶)NHC(O)R⁵.

50. The compound of claim 49, or a tautomer thereof, or a pharmaceutically acceptable salt thereof, wherein R⁴ is —C₁₋₆alkyl.

51. The compound of claim 1, or a tautomer thereof, or a pharmaceutically acceptable salt thereof, wherein X¹ is —SO₂; a is 1 and R¹ is halogen.

52. The compound of claim 51, or a tautomer thereof, or a pharmaceutically acceptable salt thereof, wherein R¹ is —Cl.

53. The compound of claim 52, or a tautomer thereof, or a pharmaceutically acceptable salt thereof, wherein R³ is an optionally substituted —C₆aryl, —C₅₋₆heteroaryl or —C₅₋₇heterocycloalkyl group.

54. The compound of claim 53, or a tautomer thereof, or a pharmaceutically acceptable salt thereof, wherein R³ is of the formula: 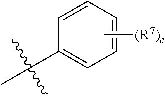
wherein,
R⁷ is independently in each occurrence selected from halogen, —OH, —CN, —NO₂, —C(O)R⁹, —C(O)N(R⁸)₂, —C(R¹⁰)═NR¹⁰, —SO₂R⁹, —SO₂N(R⁸)₂, —N(R¹⁰)C(O)R⁹, —N(R¹⁰)SO₂R⁹, —N(R¹⁰)C(O)N(R⁸)₂, —N(R¹⁰)SO₂N(R⁸)₂, —N(R⁸)₂, —P(O)(R⁸)₂, —P(O)(OR⁸)₂, —B(OR⁸)₂, —C₁₋₆alkyl, —C₂₋₆alkenyl, —C₂₋₆alkynyl, —OC₁₋₆alkyl, —C₆-10aryl, —C₅₋₁₀heteroaryl, —C₃₋₁₀cycloalkyl, and —C₄₋₁₀heterocycloalkyl;
R⁷ is independently in each occurrence selected from —H, —C₁₋₆alkyl, —C₂₋₆alkenyl, —C₂₋₆alkynyl, —C₃₋₁₀cycloalkyl, —C₄₋₁₀heterocycloalkyl, —C₆aryl, and —C₅₋₁₀heteroaryl, or two R⁸ are taken together with their adjacent atom(s) to form a C₄₋₁₀heterocycloalkyl group;
R⁹ is independently in each occurrence selected from —C₁₋₆alkyl, —C₂₋₆alkenyl, —C₂₋₆alkynyl, —C₃₋₇cycloalkyl, —C₆aryl, and —C₅₋₆heteroaryl; and
R¹⁰ is independently in each occurrence selected from —H, —C₁₋₆alkyl, —C₂₋₆alkenyl, —C₂₋₆alkynyl, —C₃₋₇cycloalkyl, —C₆aryl, and —C₅₋₆heteroaryl; and
c is 0, 1, 2, 3, 4 or 5;
wherein said alkyl, alkenyl, alkynyl, cycloalkyl, heterocycloalkyl, aryl or heteroaryl group is optionally further substituted.

55. The compound of claim 53, or a tautomer thereof, or a pharmaceutically acceptable salt thereof, wherein R³ is of the formula: 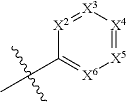
wherein,
X², X³, X⁴, X⁵, and X⁶ are each independently selected from N and CR¹¹, wherein at most three of X², X³, X⁴, X⁵, and X⁶ are N;
R⁸ is independently in each occurrence selected from —H, —C₁₋₆alkyl, —C₂₋₆alkenyl, —C₂₋₆alkynyl, —C₃₋₁₀cycloalkyl, —C₄₋₁₀heterocycloalkyl, —C₆aryl, and —C₅₋₁₀heteroaryl, or two R⁸ are taken together with their adjacent atom(s) to form a C₄₋₁₀heterocycloalkyl group;
R⁹ is independently in each occurrence selected from —C₁₋₆alkyl, —C₂₋₆alkenyl, —C₂₋₆alkynyl, —C₃₋₇cycloalkyl, —C₆aryl, and —C₅₋₆heteroaryl;
R¹⁰ is independently in each occurrence selected from —H, —C₁₋₆alkyl, —C₂₋₆alkenyl, —C₂₋₆alkynyl, —C₃₋₇cycloalkyl, —C₆aryl, and —C₅₋₆heteroaryl;
R” is independently in each occurrence selected from hydrogen, halogen, —OH, —CN, —NO₂, —C(O)R⁹, —C(O)N(R⁸)₂, —C(R¹⁰)═NR¹⁰, —SO₂R⁹, —SO₂N(R⁸)₂, —N(R¹⁰)C(O)R⁹, —N(R¹⁰)SO₂R⁹, —N(R¹⁰)C(O)N(R⁸)₂, —N(R¹⁰)SO₂N(R⁸)₂, —N(R⁸)₂, —P(O)(R⁸)₂, —P(O)(OR⁸)₂, —B(OR⁸)₂, —C₁₋₆alkyl, —C₂₋₆alkenyl, —C₂₋₆alkynyl, —OC₁₋₆alkyl, —C₆-10aryl, —C₅₋₁₀heteroaryl, —C₃₋₁₀cycloalkyl, and —C₄₋₁₀heterocycloalkyl; and
wherein said alkyl, alkenyl, alkynyl, cycloalkyl, heterocycloalkyl, aryl or heteroaryl group is optionally further substituted.

56. The compound of claim 53, or a tautomer thereof, or a pharmaceutically acceptable salt thereof, wherein R³ is of the formula: 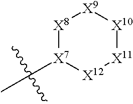
wherein,
X⁷ is selected from N and CR”;
X⁸, X⁹, X¹⁰, X”, and X¹² are each independently selected from O, NR^{x} and C(R^{Y})₂, wherein at most two of X⁷, X⁸, X⁹, X¹⁰, X”, and X¹² are or comprise O or N;
R⁸ is independently in each occurrence selected from —H, —C₁₋₆alkyl, —C₂₋₆alkenyl, —C₂₋₆alkynyl, —C₃₋₁₀cycloalkyl, —C₄₋₁₀heterocycloalkyl, —C₆aryl, and —C₅₋₁₀heteroaryl, or two R⁸ are taken together with their adjacent atom(s) to form a C₄₋₁₀heterocycloalkyl group;
R⁹ is independently in each occurrence selected from —C₁₋₆alkyl, —C₂₋₆alkenyl, —C₂₋₆alkynyl, —C₃₋₇cycloalkyl, —C₆aryl, and —C₅₋₆heteroaryl; and
R¹⁰ is independently in each occurrence selected from —H, —C₁₋₆alkyl, —C₂₋₆alkenyl, —C₂₋₆alkynyl, —C₃₋₇cycloalkyl, —C₆aryl, and —C₅₋₆heteroaryl;
R” is independently in each occurrence selected from hydrogen, halogen, —OH, —CN, —NO₂, —C(O)R⁹, —C(O)N(R⁸)₂, —C(R¹⁰)═NR¹⁰, —SO₂R⁹, -SO₂N(R⁸)₂, —N(R¹⁰)C(O)R⁹, —N(R¹⁰)S02R⁹, —N(R¹⁰)C(O)N(R⁸)₂, —N(R¹⁰)SO₂N(R⁸)₂, —N(R⁸)₂, —P(O)(R⁸)₂, —P(O)(OR⁸)₂, —B(OR⁸)₂, —C₁₋₆alkyl, —C₂₋₆alkenyl, —C₂₋₆alkynyl, —OC₁₋₆alkyl, —C₆-10aryl,-C₅₋₁₀heteroaryl, —C₃₋₁₀cycloalkyl, and —C₄₋₁₀heterocycloalkyl;
R^{x} is independently in each occurrence selected from hydrogen, —C(O)R⁹, —C(O)N(R⁸)₂, —SO₂R⁹, —SO₂N(R⁸)₂, —P(O)(R⁸)₂, —P(O)(OR⁸)₂, —B(OR⁸)₂, —C₁₋₆alkyl, —C₂₋₆alkenyl, —C₂₋₆alkynyl, —C₆-10aryl, —C₅₋₁₀heteroaryl, —C₃₋₁₀cycloalkyl, and —C₄₋₁₀heterocycloalkyl;
R^{y} is independently in each occurrence selected from hydrogen, halogen, —OH, —CN, —NO₂, —C(O)R⁹, —C(O)N(R⁸)₂, —C(R¹⁰)═NR¹⁰, —SO₂R⁹, —SO₂N(R⁸)₂, —N(R¹⁰)C(O)R⁹, —N(R¹⁰)SO₂R⁹, —N(R¹⁰)C(O)N(R⁸)₂, —N(R¹⁰)SO₂N(R⁸)₂, —N(R⁸)₂, —P(O)(R⁸)₂, —P(O)(OR⁸)₂, —B(OR⁸)₂, —C₁₋₆alkyl, —C₂₋₆alkenyl, —C₂₋₆alkynyl, —OC₁₋₆alkyl, —C₆-10aryl,-C₅₋₁₀heteroaryl, —C₃₋₁₀cycloalkyl, and —C₄₋₁₀heterocycloalkyl or two R^{y} are taken together with their adjacent atom(s) to form a C₄₋₁₀cycloalkyl or C₄₋₁₀heterocycloalkyl group; and
wherein said alkyl, alkenyl, alkynyl, cycloalkyl, heterocycloalkyl, aryl or heteroaryl group is optionally further substituted.

57. The compound of claim 55, or a tautomer thereof, or a pharmaceutically acceptable salt thereof, wherein R” in is a halogen or halogenated —C₁₋₆alkyl or wherein R” or R^{y} is a halogen or halogenated —C₁₋₆alkyl, in at least one occurrence.

58. The compound of claim 56, or a tautomer thereof, or a pharmaceutically acceptable salt thereof, wherein R” is a halogen or halogenated —C₁₋₆alkyl or wherein R” or R^{y} is a halogen or halogenated —C₁₋₆alkyl, in at least one occurrence.

59. The compound of claim 55, or a tautomer thereof, or a pharmaceutically acceptable salt thereof, wherein R” is a hydrogen atom or wherein R” or R^{y} is a hydrogen atom in each occurrence.

60. The compound of claim 56, or a tautomer thereof, or a pharmaceutically acceptable salt thereof, wherein R” is a hydrogen atom or wherein R” or R^{y} is a hydrogen atom in each occurrence.

61. The compound of claim 1, or a tautomer thereof, or a pharmaceutically acceptable salt thereof, wherein R² is of the formula: 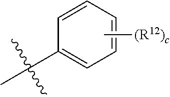
wherein,
R¹² is independently in each occurrence selected from halogen, —OH, —CN, —NO₂, —C(O)R¹⁴, —C(O)N(R¹³)₂, —C(R¹⁵)═NR¹⁵, —SO₂R¹⁴, —SO₂N(R¹³)₂, —N(R¹⁵)C(O)R¹⁴, —N(R¹⁵)SO₂R¹⁴, —N(R¹⁵)C(O)N(R¹³)₂, —N(R¹⁵)SO₂N(R¹³)₂, —N(R¹³)₂, —P(O)(R¹³)₂, —P(O)(OR¹³)₂, —B(OR¹³)₂, —C₁₋₆alkyl, —C₂₋₆alkenyl, —C₂₋₆alkynyl, —OC₁₋₆alkyl, —C₆-10aryl, —C₅₋₁₀heteroaryl, —C₃₋₁₀cycloalkyl, and —C₄₋₁₀heterocycloalkyl;
R¹³ is independently in each occurrence selected from —H, —C₁₋₆alkyl, —C₂₋₆alkenyl, —C₂₋₆alkynyl, —C₃₋₁₀cycloalkyl, —C₄₋₁₀heterocycloalkyl, —C₆aryl, and —C₅₋₁₀heteroaryl, or two R¹³ are taken together with their adjacent atom(s) to form a C₄₋₁₀heterocycloalkyl group;
R¹⁴ is independently in each occurrence selected from —C₁₋₆alkyl, —C₂₋₆alkenyl, —C₂₋₆alkynyl, —C₃₋₇cycloalkyl, —C₆aryl, and —C₅₋₆heteroaryl; and
R¹⁵ is independently in each occurrence selected from —H, —C₁₋₆alkyl, —C₂₋₆alkenyl, —C₂₋₆alkynyl, —C₃₋₇cycloalkyl, —C₆aryl, and —C₅₋₆heteroaryl; and
c is 0, 1, 2, 3, 4 or 5;
wherein said alkyl, alkenyl, alkynyl, cycloalkyl, heterocycloalkyl, aryl or heteroaryl group is optionally further substituted;
or
wherein R² is of the formula: 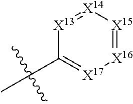
wherein,
X¹³, X¹⁴, X¹, X¹⁶, and X¹⁷ are each independently selected from N and CR¹⁶, wherein at most three of X1, X¹⁴, X1, X¹⁶, and X¹⁷ are N;
R¹³ is independently in each occurrence selected from —H, —C₁₋₆alkyl, —C₂₋₆alkenyl, —C₂₋₆alkynyl, —C₃₋₁₀cycloalkyl, —C₄₋₁₀heterocycloalkyl, —C₆aryl, and —C₅₋₁₀heteroaryl, or two R¹³ are taken together with their adjacent atom(s) to form a C₄₋₁₀heterocycloalkyl group;
R¹⁴ is independently in each occurrence selected from —C₁₋₆alkyl, —C₂₋₆alkenyl, —C₂₋₆alkynyl, —C₃₋₇cycloalkyl, —C₆aryl, and —C₅₋₆heteroaryl; and
R¹⁵ is independently in each occurrence selected from —H, —C₁₋₆alkyl, —C₂₋₆alkenyl, —C₂₋₆alkynyl, —C₃₋₇cycloalkyl, —C₆aryl, and —C₅₋₆heteroaryl;
R¹⁶ is independently in each occurrence selected from hydrogen, halogen, —OH, —CN, —NO₂, —C(O)R¹⁴, —C(O)N(R¹³)₂, —C(R¹⁵)═NR¹⁵, —SO₂R¹⁴, —SO₂N(R¹³)₂, —N(R¹⁵)C(O)R¹⁴, —N(R¹⁵)SO₂R¹⁴, —N(R¹⁵)C(O)N(R¹³)₂, —N(R¹⁵)SO₂N(R¹³)₂, —N(R¹³)₂, —P(O)(R¹³)₂, —P(O)(OR¹³)₂, —B(OR¹³)₂, —C₁₋₆alkyl, —C₂₋₆alkenyl, —C₂₋₆alkynyl, —OC₁₋₆alkyl, —C₆-10aryl, —C₅₋₁₀heteroaryl, —C₃₋₁₀cycloalkyl, and —C₄₋₁₀heterocycloalkyl; and
wherein said alkyl, alkenyl, alkynyl, cycloalkyl, heterocycloalkyl, aryl or heteroaryl group is
optionally further substituted;
or
wherein R² is of the formula: 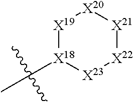
wherein,
X¹⁸ is selected from N and CR¹⁶;
X¹⁹, X²⁰, X²¹, X²², and X²³ are each independently selected from O, NR^{W} and C(R^{z})₂, wherein at most two of X1⁸, X¹⁹, X²⁰, X²¹, X²², and X²³ are or comprise 0 or N;
R¹³ is independently in each occurrence selected from —H, —C₁₋₆alkyl, —C₂₋₆alkenyl, —C₂₋₆alkynyl, —C₃₋₁₀cycloalkyl, —C₄₋₁₀heterocycloalkyl, —C₆aryl, and —C₅₋₁₀heteroaryl, or two R¹³ are taken together with their adjacent atom(s) to form a C₄₋₁₀heterocycloalkyl group;
R¹⁴ is independently in each occurrence selected from —C₁₋₆alkyl, —C₂₋₆alkenyl, —C₂₋₆alkynyl, —C₃₋₇cycloalkyl, —C₆aryl, and —C₅₋₆heteroaryl; and
R¹⁵ is independently in each occurrence selected from —H, —C₁₋₆alkyl, —C₂₋₆alkenyl, —C₂₋₆alkynyl, —C₃₋₇cycloalkyl, —C₆aryl, and —C₅₋₆heteroaryl;
R¹⁶ is independently in each occurrence selected from hydrogen, halogen, —OH, —CN, —NO₂, —C(O)R¹⁴, —C(O)N(R¹³)₂, —C(R¹⁵)═NR¹⁵, —SO₂R¹⁴, —SO₂N(R¹³)₂, —N(R¹⁵)C(O)R¹⁴, —N(R¹⁵)SO₂R¹⁴, —N(R¹⁵)C(O)N(R¹³)₂, —N(R¹⁵)SO₂N(R¹³)₂, —N(R¹³)₂, —P(O)(R¹³)₂, —P(O)(OR¹³)₂, —B(OR¹³)₂, —C₁₋₆alkyl, —C₂₋₆alkenyl, —C₂₋₆alkynyl, —OC₁₋₆alkyl, —C₆-10aryl, —C₅₋₁₀heteroaryl, —C₃₋₁₀cycloalkyl, and —C₄₋₁₀heterocycloalkyl;
R^{W} is independently in each occurrence selected from hydrogen, —C(O)R¹⁴, —C(O)N(R¹³)₂, —SO₂R¹⁴, —SO₂N(R¹³)₂, —P(O)(R¹³)₂, —P(O)(OR¹³)₂, —B(OR¹³)₂, —C₁₋₆alkyl, —C₂₋₆alkenyl, —C₂₋₆alkynyl, —C₆-10aryl, —C₅₋₁₀heteroaryl, —C₃₋₁₀cycloalkyl, and —C₄₋₁₀heterocycloalkyl;
R^{z} is independently in each occurrence selected from hydrogen, halogen, —OH, —CN, —NO₂, —C(O)R¹⁴, —C(O)N(R¹³)₂, —C(R¹⁵)═NR¹⁵, —SO₂R¹⁴, —SO₂N(R¹³)₂, —N(R¹⁵)C(O)R¹⁴, —N(R¹⁵)SO₂R¹⁴, —N(R¹⁵)C(O)N(R¹³)₂, —N(R¹⁵)SO₂N(R¹³)₂, —N(R¹³)₂, —P(O)(R¹³)₂, —P(O)(OR¹³)₂, —B(OR¹³)₂, —C₁₋₆alkyl, —C₂₋₆alkenyl, —C₂₋₆alkynyl, —OC₁₋₆alkyl, —C₆-10aryl, —C₅₋₁₀heteroaryl, —C₃₋₁₀cycloalkyl, and —C₄₋₁₀heterocycloalkyl or two R^{z} are taken together with their adjacent atom(s) to form a C₄₋₁₀cycloalkyl or C₄₋₁₀heterocycloalkyl group; and
wherein said alkyl, alkenyl, alkynyl, cycloalkyl, heterocycloalkyl, aryl or heteroaryl group is optionally further substituted.

62. The compound of claim 61, or a tautomer thereof, or a pharmaceutically acceptable salt thereof, wherein R¹⁶ or R^{z} is a hydrogen atom, a halogen, or halogenated —C₁₋₆alkyl, in at least one occurrence.

63. The compound of claim 1, wherein the compound is selected from: 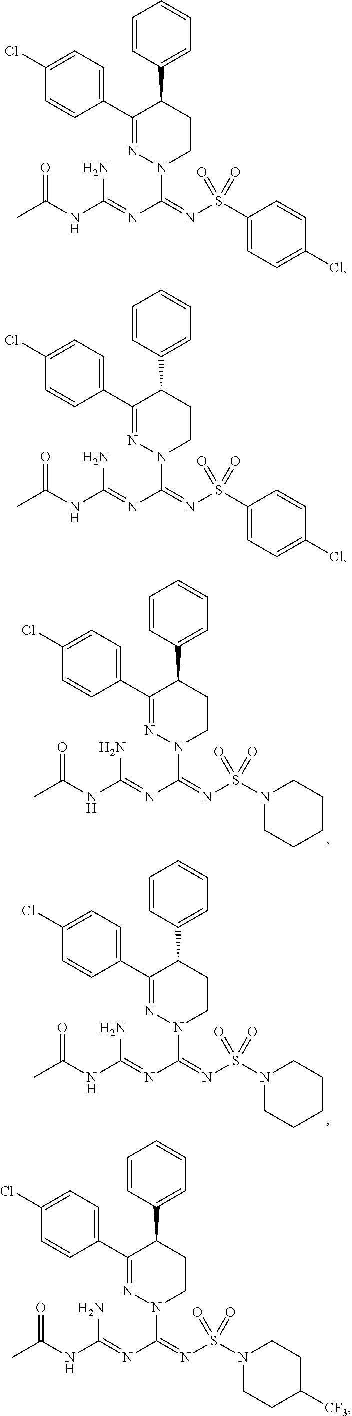 [SMILES: CC(=O)N/C(N)=N/C(=N/S(=O)(=O)c1ccc(Cl)cc1)N1CC[C@H](c2ccccc2)C(c2ccc(Cl)cc2)=N1 ; CC(=O)N/C(N)=N/C(=N/S(=O)(=O)c1ccc(Cl)cc1)N1CC[C@@H](c2ccccc2)C(c2ccc(Cl)cc2)=N1 ; CC(=O)N/C(N)=N/C(=N/S(=O)(=O)N1CCCCC1)N1CC[C@H](c2ccccc2)C(c2ccc(Cl)cc2)=N1 ; CC(=O)N/C(N)=N/C(=N/S(=O)(=O)N1CCCCC1)N1CC[C@@H](c2ccccc2)C(c2ccc(Cl)cc2)=N1 ; CC(=O)N/C(N)=N/C(=N/S(=O)(=O)N1CCC(C(F)(F)F)CC1)N1CC[C@H](c2ccccc2)C(c2ccc(Cl)cc2)=N1] 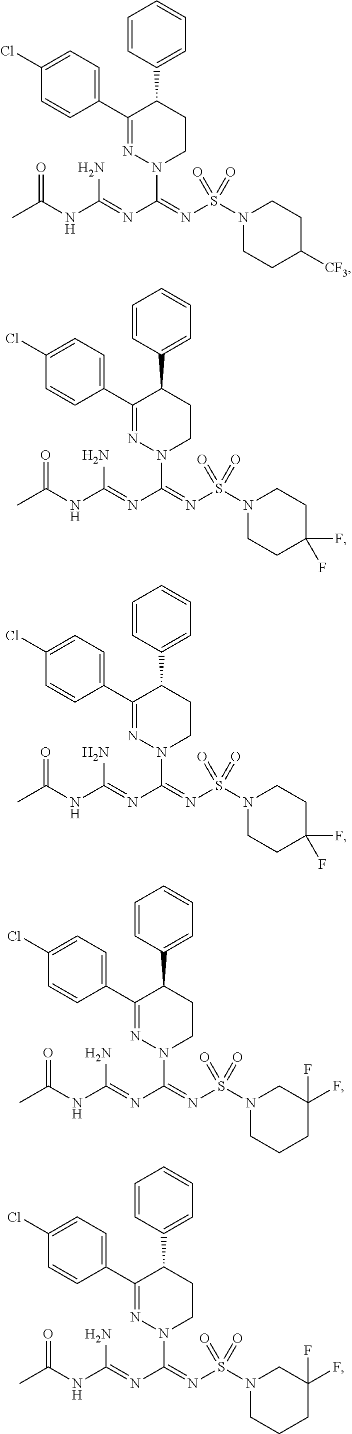 [SMILES: CC(=O)N/C(N)=N/C(=N/S(=O)(=O)N1CCC(C(F)(F)F)CC1)N1CC[C@@H](c2ccccc2)C(c2ccc(Cl)cc2)=N1 ; CC(=O)N/C(N)=N/C(=N/S(=O)(=O)N1CCC(F)(F)CC1)N1CC[C@H](c2ccccc2)C(c2ccc(Cl)cc2)=N1 ; CC(=O)N/C(N)=N/C(=N/S(=O)(=O)N1CCC(F)(F)CC1)N1CC[C@@H](c2ccccc2)C(c2ccc(Cl)cc2)=N1 ; CC(=O)N/C(N)=N/C(=N/S(=O)(=O)N1CCCC(F)(F)C1)N1CC[C@H](c2ccccc2)C(c2ccc(Cl)cc2)=N1 ; CC(=O)N/C(N)=N/C(=N/S(=O)(=O)N1CCCC(F)(F)C1)N1CC[C@@H](c2ccccc2)C(c2ccc(Cl)cc2)=N1] 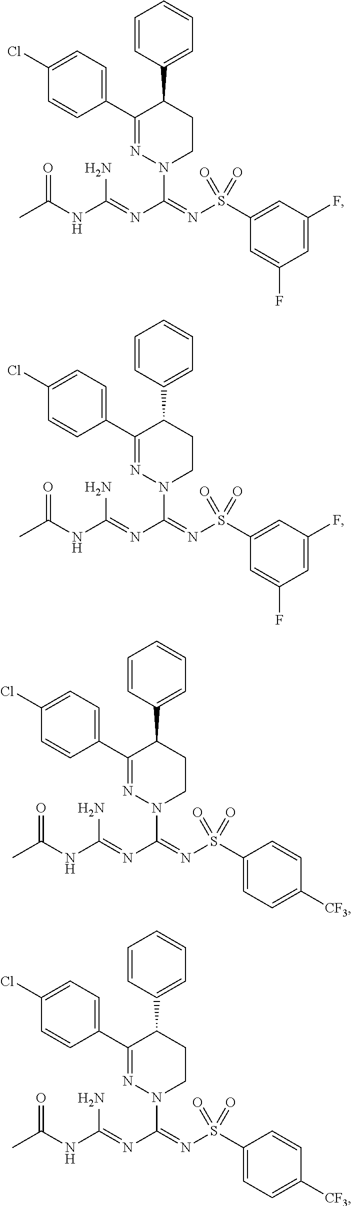 [SMILES: CC(=O)N/C(N)=N/C(=N/S(=O)(=O)c1cc(F)cc(F)c1)N1CC[C@H](c2ccccc2)C(c2ccc(Cl)cc2)=N1 ; CC(=O)N/C(N)=N/C(=N/S(=O)(=O)c1cc(F)cc(F)c1)N1CC[C@@H](c2ccccc2)C(c2ccc(Cl)cc2)=N1 ; CC(=O)N/C(N)=N/C(=N/S(=O)(=O)c1ccc(C(F)(F)F)cc1)N1CC[C@H](c2ccccc2)C(c2ccc(Cl)cc2)=N1 ; CC(=O)N/C(N)=N/C(=N/S(=O)(=O)c1ccc(C(F)(F)F)cc1)N1CC[C@@H](c2ccccc2)C(c2ccc(Cl)cc2)=N1] 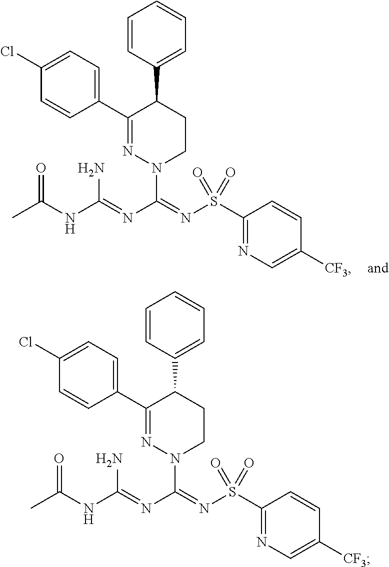 [SMILES: CC(=O)N/C(N)=N/C(=N/S(=O)(=O)c1ccc(C(F)(F)F)cn1)N1CC[C@H](c2ccccc2)C(c2ccc(Cl)cc2)=N1 ; CC(=O)N/C(N)=N/C(=N/S(=O)(=O)c1ccc(C(F)(F)F)cn1)N1CC[C@@H](c2ccccc2)C(c2ccc(Cl)cc2)=N1]
or a tautomer thereof, or a pharmaceutically acceptable salt thereof.

64. A pharmaceutical composition comprising a compound of claim 1, or a tautomer thereof, or a pharmaceutically acceptable salt thereof, together with a pharmaceutically acceptable carrier, diluent, or excipient.

65. A method of treating a disorder related to appetite or one of its complications, a disorder related to glucose regulation or one of its complications, a fibrosis related disorder or one of its complications, a disorder related to metabolism or one of its complications, a disorder related to skin and hair growth and healing, a disorder related to the GI tract, and a disorder related to obesity or one of its complications, or a combination thereof comprising administering a compound according to claim 1, or a tautomer thereof, or a pharmaceutically acceptable salt thereof, to a patient in need thereof.

66. A method of treating diabetes, obesity, or chronic kidney disease comprising administering a compound according to claim 1, or a tautomer thereof, or a pharmaceutically acceptable salt thereof.
